SEQUENCE LISTING (15 sequences)

SEQ ID NO: 1 - DNA, 1216 bp, Homo sapiens
  gene (1)..(1216): CXCL13 Hs.100431
ttcggcacttgggagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccagggagaaagaacttccccatacaaataaggcatgaggactatgtgaaaaataaccttgcaggagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtgggggcggggccggggggactctggtatctaattctttaatgattcctataaatctaatgacattcaataaagttgagcaaacattttactt

SEQ ID NO: 2 - DNA, 3198 bp, Homo sapiens
  gene (1)..(3198): NELL2 Hs. 79389 (arch.); Hs. 505326 (current)
ttgggaggagcagtctctccgctcgtctcccggagctttctccattgtctctgcctttacaacagagggagacgatggactgagctgatccgcaccatggagtctcgggtcttactgagaacattctgtttgatcttcggtctcggagcagtttgggggcttggtgtggacccttccctacagattgacgtcttaacagagttagaacttggggagtccacgaccggagtgcgtcaggtcccggggctgcataatgggacgaaagcctttctctttcaagatactcccagaagcataaaagcatccactgctacagctgaacagttttttcagaagctgagaaataaacatgaatttactattttggtgaccctaaaacagacccacttaaattcaggagttattctctcaattcaccacttggatcacaggtacctggaactggaaagtagtggccatcggaatgaagtcagactgcattaccgctcaggcagtcaccgccctcacacagaagtgtttccttacattttggctgatgacaagtggcacaagctctccttagccatcagtgcttcccatttgattttacacattgactgcaataaaatttatgaaagggtagtagaaaagccctccacagacttgcctctaggcacaacattttggctaggacagagaaataatgcgcatggatattttaagggtataatgcaagatgtccaattacttgtcatgccccagggatttattgctcagtgcccagatcttaatcgcacctgtccaacttgcaatgacttccatggacttgtgcagaaaatcatggagctacaggatattttagccaaaacatcagccaagctgtctcgagctgaacagcgaatgaatagattggatcagtgctattgtgaaaggacttgcaccatgaagggaaccacctaccgagaatttgagtcctggatagacggctgtaagaactgcacatgcctgaatggaaccatccagtgtgaaactctaatctgcccaaatcctgactgcccacttaagtcggctcttgcgtatgtggatggcaaatgctgtaaggaatgcaaatcgatatgccaatttcaaggacgaacctactttgaaggagaaagaaatacagtctattcctcttctggagtatgtgttctctatgagtgcaaggaccagaccatgaaacttgttgagagttcaggctgtccagctttggattgtccagagtctcatcagataaccttgtctcacagctgttgcaaagtttgtaaaggttatgacttttgttctgaaaggcataactgcatggagaattccatctgcagaaatctgaatgacagggctgtttgtagctgtcgagatggttttagggctcttcgagaggataatgcctactgtgaagacatcgatgagtgtgctgaagggcgccattactgtcgtgaaaatacaatgtgtgtcaacaccccgggttcttttatgtgcatctgcaaaactggatacatcagaattgatgattattcatgtacagaacatgatgagtgtatcacaaatcagcacaactgtgatgaaaatgctttatgcttcaacactgttggaggacacaactgtgtttgcaagccgggctatacagggaatggaacgacatgcaaagcattttgcaaagatggctgtaggaatggaggagcctgtattgccgctaatgtgtgtgcctgcccacaaggcttcactggacccagctgtgaaacggacattgatgaatgctctgatggttttgttcaatgtgacagtcgtgctaattgcattaacctgcctggatggtaccactgtgagtgcagagatggctaccatgacaatgggatgttttcaccaagtggagaatcgtgtgaagatattgatgagtgtgggaccgggaggcacagctgtgccaatgataccatttgcttcaatttggatggcggatatgattgtcgatgtcctcatggaaagaattgcacaggggactgcatccatgatggaaaagttaagcacaatggtcagatttgggtgttggaaaatgacaggtgctctgtgtgctcatgtcagaatggattcgttatgtgtcgacggatggtctgtgactgtgagaatcccacagttgatcttttttgctgccctgaatgtgacccaaggcttagtagtcagtgcctccatcaaaatggggaaactttgtataacagtggtgacacctgggtccagaattgtcaacagtgccgctgcttgcaaggggaagttgattgttggcccctgccttgcccagatgtggagtgtgaattcagcattctcccagagaatgagtgctgcccgcgctgtgtcacagacccttgccaggctgacaccatccgcaatgacatcaccaagacttgcctggacgaaatgaatgtggttcgcttcaccgggtcctcttggatcaaacatggcactgagtgtactctctgccagtgcaagaatggccacatctgttgctcagtggatccacagtgccttcaggaactgtgaagttaactgtctcatgggagatttctgttaaaagaatgttctttcattaaaagaccaaaaagaagttaaaacttaaattgggtgatttgtgggcagctaaatgcagctttgttaatagctgagtgaactttcaattatgaaatttgtggagcttgacaaaatcacaaaaggaaaattactggggcaaaattagacctcaagtctgcctctactgtgtctcacatcaccatgtagaagaatgggcgtacagtatataccgtgacatcctgaaccctggatagaaagcctgagcccattggatctgtgaaagcctctagcttcactggtgcagaaaattttcctctagatcagaatcttcagaatcagttaggttcctcactgcaagaaataaaatgtcaggcagtgaatgaattatattttcagaagtaaagcaaagaagctataacatgttatgtacagtacactctgaaaagaaatctgaaacaagttattgtaatgataaaaataatgcacaggcatggttacttaatattttctaacaggaaaagtcatccctatttccttgttttactgcacttaatattatttggttgaatttgttcagtataagctcgttcttgtgcaaaattaaataaatatttctcttacctt

SEQ ID NO: 3 - DNA, 2963 bp, Homo sapiens
  gene (1)..(2963): SH3; Unigene Hs.56045
gttcctccgggagcccaacaccgttcccgcgcggccacgatgatccctccgagcagcccccgcgaggacggcgtggacgggctgcccaaggaggcggtgggcgccgagcaaccgccctctcctgcatccaccagcagccaggaatccaagctccagaaactaaaacgatcactttctttcaagaccaagagtttacggagcaaaagtgctgacaacttcttccagcgaaccaacagcgaagacatgaaactgcaagcacacatggtggctgagatcagccccagctccagcccactccctgctccaggaagcctgacgtccacacccgccagggctggtctgcatccaggtggcaaggctcatgcctttcaggaatacatcttcaagaagcccactttctgtgatgtctgcaaccacatgatagtgggaacaaatgctaagcatggactgcgctgcaaagcctgtaagatgagcatccaccacaagtgcacagatggcctggcaccccagcggtgcatgggcaagctgccaaaggggtttcggcgttactacagctcccccttgctcattcatgaacagtttggctgcattaaagaagttatgcccattgcctgtggcaataaggtggaccctgtctacgagaccctccgcttcggcacctccctggcccagaggacaaagaagggcagctccggcagtggctctgactcacctcacagaacctctacttcagatcttgtggaggttcctgaggaagccaatgggccaggaggcgggtatgacctaaggaaacgcagcaacagcgtgtttacatatccagaaaatggcactgatgatttcagagatccagcgaagaacataaaccaccagggatctctttccaaagacccattacagatgaacacctatgttgccttgtacaaatttgtaccacaggagaatgaagatttggaaatgaggccaggagacataattactcttttagaggattccaatgaagactggtggaaagggaaaattcaagacagaattggcttctttccagccaactttgttcagagactacaacaaaatgagaagatttttagatgtgttagaaccttcattgggtgtaaggaacaggggcagataacactgaaagagaatcagatctgcgtgagttctgaagaagaacaagatggttttatcagagtcctcagtggaaaaaagaaaggcctcatcccccttgatgtactagaaaacatctgattgctggctcctcctccgtttgcagtaggcaagctctgctgcgatgcctctgcctcatctcacactgcgtcaacccaaaggagctgccgcactgacccagccccccaggaaacagtgagacaagaatcaagtatctgagactgtggagtaatagccacaaaacagagggcccactgcacagcatatccaggctgccacaggtggggacgaggctgagagagtcagcaggcagagccagatgccatgcttggcagcagcagtaggactataaaccacagctgtcccccaggatcccactcctttcctgtctgtgtggtgtaagttaacacactggagtgtgctccagtttgcagggtagcccagtgcaaggttcagatccatgtagctaagtattatcctgcttccagacctatgtcaccagtaccaatcagtcagtgtcatcacatttcaggccccaagcaatctctgtgcaaagcatcagaaagacctgcttcccagcccccagcattccagtgctctccaggcttcctctctttgtgattgtgctgtccagagtgtccagcttgttctttctttctcttcagtcctctgagtacatctggtggtgtgcattagatgtgagggctatgttgacatggcatcacctccaaagacctgacctgcctaaagactgatgacaggccatccttcctgctgttctaggtactggcctgggtgacagagcaggacatgagacatagatacagtggggaggagaagtggggaaaggtggagcagagagttcttacttattgaagattatacagccctttcggttatgaagtccctgcttgaaggcaatggacctggggaagagactatcacaaaaagtctccattttcattttacatcctctctattggaggcagcacttttccctcatgctgtcctataggactccactttgaaggttgtgcctacgttgcagggaactaggaacatggaggggaaccaacaacagcatcttagaagaaatgtagccaaattggagtccattcttctttagggcagtatatgaaatcctagcagatgtaaaatggaaaagaatcctaatgcttcttccttcagaaagtagaggaactaggggcccaattagcatcatctaggggaatctctattactctgtacttatactaatgtttacaagaatgcaatatactgtgatgccttcctactcaagcctcctagcattcaaacttccatcctattagtcattaacgtggttaaacttcaattcacaatcaccttggaatcaatgtcagtttgatttattttgttacagagcaataaaatcattagaacaatggtttttaaaagacttaagtggatgcatcctatgcatgtaagcattatttcccaaaccaaagcatcattcatctccatttatttcttttgttcccgctcagtgtgaagttgggaactgagaggggatggctgctggtttcacagaagtttggatgccttactcttatcttaaagccagcatccagaatttcctccctctctgatgccacatgcaaaaccaggtgtacgatgtcaagatggattctttgagagccagggtagatcatatgactgcctttctgtaaaattggatgcctagtacaaatgctctttgcctctaattcagtattccatttaataaaaacaatacagtggctggaaaggagcatcag

SEQ ID NO: 4 - DNA, 2313 bp, Homo sapiens
  gene (1)..(2281): FBLN1   Unigene  Hs.79732  (arch.)  Hs. 24601  (current)
ctcctcccgggcgggataattgaacggcgcggccctggcccagcgttggctgccgaggctcggccggagcgtggagcccgcgccgctgccccaggaccgcgcccgcgcctttgtccgccgccgcccaccgcccgtcgcccgccgcccatggagcgcgccgcgccgtcgcgccgggtcccgcttccgctgctgctgctcggcggccttgcgctgctggcggccggagtggacgcggatgtcctcctggaggcctgctgtgcggacggacaccggatggccactcatcagaaggactgctcgctgccatatgctacggaatccaaagaatgcaggatggtgcaggagcagtgctgccacagccagctggaggagctgcactgtgccacgggcatcagcctggccaacgagcaggaccgctgtgccacgccccacggtgacaacgccagcctggaggccacatttgtgaagaggtgctgccattgctgtctgctggggagggcggcccaggcccagggccagagctgcgagtacagcctcatggttggctaccagtgtggacaggtcttccgggcatgctgtgtcaagagccaggagaccggagatttggatgtcgggggcctccaagaaacggataagatcattgaggttgaggaggaacaagaggacccatatctgaatgaccgctgccgaggaggcgggccctgcaagcagcagtgccgagacacgggtgacgaggtggtctgctcctgcttcgtgggctaccagctgctgtctgatggtgtctcctgtgaagatgtcaatgaatgcatcacgggcagccacagctgccggcttggagaatcctgcatcaacacagtgggctctttccgctgccagcgggacagcagctgcgggactggctatgagctcacagaggacaatagctgcaaagatattgacgagtgtgagagtggtattcataactgcctccccgattttatctgtcagaatactctgggatccttccgctgccgacccaagctacagtgcaagagtggctttatacaagatgctctaggcaactgtattgatatcaatgagtgtttgagtatcagtgccccgtgccctatcgggcatacatgcatcaacacagagggctcctacacgtgccagaagaacgtgcccaactgtggccgtggctaccatctcaacgaggagggaacgcgctgtgttgatgtggacgagtgcgcgccacctgctgagccctgtgggaagggacatcgctgcgtgaactctcccggcagtttccgctgcgaatgcaagacgggttactattttgacggcatcagcaggatgtgtgtcgatgtcaacgagtgccagcgctaccccgggcgcctgtgtggccacaagtgcgagaacacgctgggctcctacctctgcagctgttccgtgggcttccggctctctgtggatggcaggtcatgtgaagacatcaatgagtgcagcagcagcccctgtagccaggagtgtgccaacgtctacggctcctaccagtgttactgccggcgaggctaccagctcagcgatgtggatggagtcacctgtgaagacatcgacgagtgcgccctgcccaccgggggccacatctgctcctaccgctgcatcaacatccctggaagcttccagtgcagctgcccctcgtctggctacaggctggcccccaatggccgcaactgccaagacattgatgagtgtgtgactggcatccacaactgctccatcaacgagacctgcttcaacatccagggcggcttccgctgcctggccttcgagtgccctgagaactaccgccgctccgcagccacccgctgtgagcgcttgccttgccatgagaatcgggagtgctccaagctgcctctgagaataacctactaccacctctctttccccaccaacatccaagcgcccgcggtggttttccgcatgggcccctccagtgctgtccccggggacagcatgcagctggccatcaccggcggcaatgaggagggctttttcaccacccggaaggtgagcccccacagtggggtggtggccctcaccaagcctgtccccgagcccagggacttgctcctgaccgtcaagatggatctctctcgccacggcaccgtcagctcctttgtggccaagcttttcatctttgtgtctgcagagctctgagcactcgcttcgcgtcgcggggtctccctcctgttgctttcctaaccctgccctccggggcgttaataaagtcttagcaagcgtcccacacagtgaaaaaaaaaaaaaaaaaaa

SEQ ID NO: 5 - DNA, 2153 bp, Homo sapiens
  gene (1)..(2153): Unigene Hs.1104; Hs.296648 (current)
ctggtatatttgtgcctgctggaggtggaattaacagtaagaaggagaaagggattgaatggacttacaggaaggatttcaagtaaattcagggaaacacatttacttgaatagtacaacctagagtattattttacactaagacgacacaaaagatgttaaagttatcaccaagctgccggacagatatatattccaacaccaaggtgcagatcagcatagatctgtgattcagaaatcaggatttgttttggaaagagctcaagggttgagaagaactcaaaagcaagtgaagattactttgggaactacagtttatcagaagatcaacttttgctaattcaaataccaaaggcctgattatcataaattcatataggaatgcataggtcatctgatcaaataatattagccgtcttctgctacatcaatgcagcaaaaactcttaacaactgtggataattggaaatctgagtttcagctttcttagaaataactactcttgacatattccaaaatatttaaaataggacaggaaaatcggtgaggatgttgtgctcagaaatgtcactgtcatgaaaaataggtaaatttgttttttcagctactgggaaactgtacctcctagaaccttaggtttttttttttttaagaggacaagaaggactaaaaatatcaacttttgcttttggacaaaaatgcatctgactgtatttttacttaagggtattgtgggtttcctctggagctgctgggttctagtgggttatgcaaaaggaggtttgggagacaatcatgttcactccagttttatttatagaagactacggaaccacgaaagacgggaaatacaaagggaaattctctctatcttgggtttgcctcacagacccagaccattttcacctggaaaacaagcgtcctctgcacctctctttatgctggatctctacaatgccatgaccaatgaagaaaatcctgaagagtcggagtactcagtaagggcatccttggcagaagagaccagaggggcaagaaagggatacccagcctctcccaatgggtatcctcgtcgcatacagttatctcggacgactcctctgaccacccagagtcctcctctagccagcctccatgataccaactttctgaatgatgctgacatggtcatgagctttgtcaacttagttgaaagagacaaggatttttctcaccagcgaaggcattacaaagaatttcgatttgatcttacccaaattcctcatggagaggcagtgacagcagctgaattccggatatacaaggaccggagcaacaaccgatttgaaaatgaaacaattaagattagcatatatcaaatcatcaaggaatacacaaatagggatgcagatctgttcttgttagacacaagaaaggcccaagctttagatgtgggttggcttgtctttgatatcactgtgaccagcaatcattgggtgattaatccccagaataatttgggcttacagctctgtgcagaaacaggggatggacgcagtatcaacgtaaaatctgctggtcttgtgggaagacagggacctcagtcaaaacaaccattcatggtggccttcttcaaggcgagtgaggtacttcttcgatccgtgagagcagccaacaaacgaaaaaatcaaaaccgcaataaatccagctctcatcaggactcctccagaatgtccagtgttggagattataacacaagtgagcaaaaacaagcctgtaagaagcacgaactctatgtgagcttccgggatctgggatggcaggactggattatagcaccagaaggatacgctgcattttattgtgatggagaatgttcttttccacttaacgcccatatgaatgccaccaaccacgctatagttcagactctggttcatctgatgtttcctgaccacgtaccaaagccttgttgtgctccaaccaaattaaatgccatctctgttctgtactttgatgacagctccaatgtcattttgaaaaaatatagaaatatggtagtacgctcatgtggctgccactaatattaaataatattgataataacaaaaagatctgtattaaggtttatggctgcaataaaaagcatactttcagacaaacagaaaaaaaaa

SEQ ID NO: 6 - DNA, 2301 bp, Homo sapiens
  gene (1)..(2301): Unigene  Hs. 89791 (arch.); Hs.567356 (curr.)
agcagagcggacgggcgcgcgggaggcgcgcagagctttcgggctgcaggcgctcgctgccgctggggaattgggctgtgggcgaggcggtccgggctggcctttatcgctcgctgggcccatcgtttgaaactttatcagcgagtcgccactcgtcgcaggaccgagcggggggcgggggcgcggcgaggcggcggccgtgacgaggcgctcccggagctgagcgcttctgctctgggcacgcatggcgcccgcacacggagtctgacctgatgcagacgcaagggggttaatatgaacgcccctctcggtggaatctggctctggctccctctgctcttgacctggctcacccccgaggtcaactcttcatggtggtacatgagagctacaggtggctcctccagggtgatgtgcgataatgtgccaggcctggtgagcagccagcggcagctgtgtcaccgacatccagatgtgatgcgtgccattagccagggcgtggccgagtggacagcagaatgccagcaccagttccgccagcaccgctggaattgcaacaccctggacagggatcacagcctttttggcagggtcctactccgaagtagtcgggaatctgcctttgtttatgccatctcctcagctggagttgtatttgccatcaccagggcctgtagccaaggagaagtaaaatcctgttcctgtgatccaaagaagatgggaagcgccaaggacagcaaaggcatttttgattggggtggctgcagtgataacattgactatgggatcaaatttgcccgcgcatttgtggatgcaaaggaaaggaaaggaaaggatgccagagccctgatgaatcttcacaacaacagagctggcaggaaggctgtaaagcggttcttgaaacaagagtgcaagtgccacggggtgagcggctcatgtactctcaggacatgctggctggccatggccgacttcaggaaaacgggcgattatctctggaggaagtacaatggggccatccaggtggtcatgaaccaggatggcacaggtttcactgtggctaacgagaggtttaagaagccaacgaaaaatgacctcgtgtattttgagaattctccagactactgtatcagggaccgagaggcaggctccctgggtacagcaggccgtgtgtgcaacctgacttcccggggcatggacagctgtgaagtcatgtgctgtgggagaggctacgacacctcccatgtcacccggatgaccaagtgtgggtgtaagttccactggtgctgcgccgtgcgctgtcaggactgcctggaagctctggatgtgcacacatgcaaggcccccaagaacgctgactggacaaccgctacatgaccccagcaggcgtcaccatccaccttcccttctacaaggactccattggatctgcaagaacactggacctttgggttctttctggggggatatttcctaaggcatgtggcctttatctcaacggaagccccctcttcctccctgggggccccaggatggggggccacacgctgcacctaaagcctaccctattctatccatctcctggtgttctgcagtcatctcccctcctggcgagttctctttggaaatagcatgacaggctgttcagccgggagggtggtgggcccagaccactgtctccacccaccttgacgtttcttctttctagagcagttggccaagcagaaaaaaaagtgtctcaaaggagctttctcaatgtcttcccacaaatggtcccaattaagaaattccatacttctctcagatggaacagtaaagaaagcagaatcaactgcccctgacttaactttaacttttgaaaagaccaagacttttgtctgtacaagtggttttacagctaccacccttagggtaattggtaattacctggagaagaatggctttcaatacccttttaagtttaaaatgtgtatttttcaaggcatttattgccatattaaaatctgatgtaacaaggtggggacgtgtgtcctttggtactatggtgtgttgtatctttgtaagagcaaaagcctcagaaagggattgctttgcattactgtccccttgatataaaaaatctttagggaatgagagttccttctcacttagaatctgaagggaattaaaaagaagatgaatggtctggcaatattctgtaactattgggtgaatatggtggaaaataatttagtggatggaatatcagaagtatatctgtacagatcaagaaaaaaaggaagaataaaattcctatatcat

SEQ ID NO: 7 - DNA, 3646 bp, Homo sapiens
  gene (1)..(3646): X11L; Unigene No. Hs.26468 (arch.); Hs.618112
gaattccatttacgcgttgctgaatctcctgtggacaaaccaccaataggccaggactgtcctgtggacagacggggtgagcctcttcttgtgtctggagattctgaaaagatttgatcaccaggagatttttcgggacattaccaaaccactcattttagtggctgcctccgggtgatgatggctgtgtgaacgactgccatggcccaccggaagcttgagagcgtggggagcggcatgttggaccatagggtgagaccaggtcctgtccctcacaggcaggagcccgagagcgaggacatggagctgcccttggagggctatgtgcccgagggcctggagctggctgccctgcggccagagagccccgcgccagaggaacaggagtgccacaaccacagccccgatggggactccagctctgactacgtgaacaacacctctgaggaggaggactatgacgagggcctccctgaggaggaggagggcatcacctactacatccgctactgccctgaggacgacagctacctagagggcatggactgcaacggggaggagtacctggcccacagtgcacaccctgtggacactgatgagtgccaggaggccgtggaggagtggacggactcggcgggcccgcacccccacggccacgaggctgaaggcagccaggactacccagaccgccaactgcccattccggagatgagccctccgtccttgaggcccatgaccaggaagaagatggtcactatgtgccagcaaagagggctaccaggactactaccccgaggaggccaacgggaacaccggcgcctccccctaccgcctgaggctggggatggggacctggaggaccaggaggaggacattgaccagatcgtggcagagatcaagatgagtctgagcatgaccagcatcaccagcgccagtgaggccagccccgagcatgggcctgagccagggcctgaggactctgtagaggcctgcccacccatcaaggccagctgcagccccagcaggcacgaggcgaggcccaagtcgctgaacctccttcccgaggccaagcaccccggagacccccagagaggcttcaagcccaagaccaggaccccagaagagaggctgaagtggccccacgagcaggtttgcaatggtctggagcagccaaggaagcagcagcgctctgatctcaatggacctgttgacaataacaacattccagagacaaagaaggtggcatcatttccaagttttgtggctgttccagggccctgcgaaccagaagacctcatcgacgggatcatctttgctgccaattacctggggtccacccagctgctatcagaacggaacccttccaaaaacatcagaatgatgcaagcgcaggaggccgtcagccgggtcaagaggatgcaaaaggctgctaagatcaagaaaaaagcgaattctgagggggatgcccagacgctgacggaagtggacctcttcatttccacccagaggatcaaggttttaaatgcagacacgcaggaaaccatgatggaccacgccttgcgtaccatctcctacatcgccgacattgggaacattgtagtgctgatggccagacgccgcatgccccggtcagcctctcaggactgcatcgagaccacgcccggggcccaggaaggcaagaagcagtataagatgatctgccatgtgttcgagtcggaggatgcccagctcatcgcccagtctatcggccaggccttcagcgtggcctaccaggagttcctgcgagccaatggcatcaaccccgaagacttgagccagaaggaatacagcgacatcatcaacacccaggagatgtacaacgacgacctcatccacttctcaaactcggagaactgcaaggagctgcagctggagaagcacaagggcgagatcctgggcgtggtggtggtggagtcgggctggggctccatcctgcccacggtgatcctggccaacatgatgaatggctgcccggctgcccgctcggggaagctgagcatcggggaccagatcatgtccatcaatggcaccagcctggtggggctgcccctcgccacctgccaaggcatcatcaagggcctgaagaaccagacacaggtgaagctcaacattgtcagctgtcccccggtcaccacggtccttatcaagcggccagacctcaagtaccagctgggcttcagcgtgcagaatggaattatctgcagcctcatgagagggggcattgctgagcgagggggcgtccgtgtgggccaccgcatcatcgagatcaacgggcagagcgtggtggccacagcccacgagaagatagtccaagctctgtccaactcggtcggagagatccacatgaagaccatgcccgccgccatgttcaggctcctcacgggtcaggagaccccgctgtacatctaggccaccccagcctggccacgcagccaggacaccgggcagggccgcccggccagaggagctgggagccgggccgcagacttgaccccgacgccacagcccagccacggacgctggctccccaaagggtgtgccctcaccacccacttgatttttttcattttgccaaaaaggggtatgtctttatcaaaggagagtcacagaacaaatgtttgtttgtaaagcgttccaagtattttgccacgttctggactgtcttctccctgcacaagccagggtgtgtctcggtagctgtgcgtggtgtggagtgtgtgtctttcctccctgaagctgtgcggagcgaactggcgcctccgagggacgcggctcccggggcagggcagccgtcacccctgcctcccgcccccttggctgggacgtctggggtcctgtggggcccccacaatggtccaaacagctgcctctgccactgactgcagggacacgggcagcctggctcccaggacacgacttgtaatgaaagtttggggacatgtgattgattgattgattgattgtaaataaaggatgatggccacaacatgaaaactccatatttatttagatgctattattactgtttggacttttattttggcaggcttttttccagactctagggttttccaatgtgactaatgaccacacctgcctctcccgtcgtctcttctgggcaccctcccacccggctgcatacccggccaggctccacagagacaggagggcacaggtgtctgcccctctttaaatcgatctacacacatccacgcacatgcgaccccgaggaaacgaaacccactctagaaaacgcgaccttggccgcacctaaagcagccagccgtgagtgcagaccccttggccagcgtggcgcagtggccctgagcagtagtggcatgtgtgtagatcaagtcggatactagtccagctcggttcattagttagcgatccatgtaatctgacgtcatcttgtctcgaagtctctttttttgcccaggccttgaagaatacactgtgacttaagaagccttaccgcggcgtaactaaagctttaggatgactgtattcgaggagtgccgtgtgttgcatgcagctacccgtaggaagacttcgcgcatatcactaataaacctgaagtcgtgatgaaaaaaaaaaaaaaaaaaaa

SEQ ID NO: 8 - DNA, 5483 bp, Homo sapiens
  gene (1)..(5483): TIMP3; Unigene No. Hs.245188 (arch.); Hs.644633 (current)
tctgtcgacttgccccagagctgatccttgtctttgtccacttctcagcgaggatggcacttcagggagcccttcccttactatcgcagagagagcaggccctccccagtcatgtccaacccagaactctgttttgttttcttcatagccctagcatcacagaaaatcaccctgtgcattcatggatgtccacgggggcaagggctttgtgttgcttaacccagcatcctgaaccgtgtttgttgaatgaatacagaaccccgtttgctctgggagagcacagaaaacagtcttctatcatatatcatagccagctcgaaacagcagatggcttccatatccagagagcaagaaccagagagagagagaaagagagagagtttgggtctttctcctctntncctgctctctccagagaaactggaggggtagcagttagcattcccccgctggttccaccaagcacagtcaaggtctctaggacatggccacccctcacctgtggaagcggtcctgctggggtgggtgggtgttagttggttctggtttgggtcagagacacccagtggcccaggtgggcgtggggccagggcgcagacgagaaggggcacgagggctccgctccgaggacccagcggcaagcaccggtcccgggngggccccagcccacccactcgcgtgcccacggcggcattattccctataaggatctgaacgatccgggggcggccccgccccgttaccccttgcccccggccccgccccctttttggagggccgatgaggtaatgcggctctgccattggtctgagggggcgggccccaacagcccgaggcggggtccccgggggcccagcgctatatcactcggccgcccaggcagacggcgcagagcgggcagcaggcaggcggcgggcgctcagacggcttctcctcctcctcttgctcctccagctcctgctccttcgccgggaggccgcccgccgagtcctggccagcgccgaggcagcctgcctgcgccccatcccgtcccgccgggcactcggagggcagcgcgccggaggccaaggttgccccgcacggcccggcgggcgagcgagctcgggctgcagcagccccgccggcgcgcacggcaactttggagaggcgagcagcagccccggcagcggcggcagcagcggcaatgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctgagcgccagaccctgccccacctcacttccctcccttcccgctgagcttcccttggacactaactcttcccagatgatgacaatgaaattagtgcctgttttcttgcaaatttagcacttggaacatttaaagaaaggtctatgctgtcatatggggtttattgggaactatcctcctggccccaccctgccccttctttttggttttgacatcattcatttccacctgggaatttctggtgccatgccagaaagaatgaggaacctgtattcctcttcttcgtgataatataatctctatttttttaggaaaacaaaaatgaaaaactactccatttgaggattgtaattcccacccctcttgcttcttccccacctcaccatctcccagaccctcttccctttgcccttctcctccaatacataaaggacacagacaaggaacttgctgaaaggccaaccatttcaggatcagtcaaaggcagcaagcagatagactcaaggtgtgtgaaagatgttatacaccaggagctgccactgcatgtcccaaccagactgtgtctgtctgtgtctgcatgtaagagtgagggagggaaggaaggaactacaagagagtcggagatgatgcagcacacacacaattccccagcccagtgatgcttgtgttgaccagatgttcctgagtctggagcaagcacccaggccagaataacagagctttcttagttggtgaagacttaaacatctgcctgaggtcaggaggcaatttgcctgccttgtacaaaagctcaggtgaaagactgagatgaatgtctttcctctccctgcctcccaccagacttcctcctggaaaacgctttggtagatttggccaggagctttcttttatgtaaattggataaatacacacaccatacactatccacagatatagccaagtagatttgggtagaggatactatttccagaatagtgtttagctcacctagggggatatgtttgtatacacatttgcatatacccacatggggacataagctaatttttttacaggacacagaattctgttcaatgctgttaaatatgccaatagtttaatctcttctattttgttgtcgttgcttgtttgaagaaaatcatgacattccaagttgacattttttttttcattttaattaaaatttgaaattctgaacaccgtcagcaccctctcttccctatcatgggtcatctgacccctgtccgtctccttgtccctgcttcatgtttgggggcctttctttaactgccttcctggcttagctcagatggcagatgagagtgtagtcaagggcctgggcacaggagggagagctgcagagtgtcctgcctgccttggctggagggacacctctcctgggtgtggagacagcttggttccctttccctagctccctggtgggtgaatgccacctcctgagatcctcacctcttggaattaaaattgttggtcactggggaaagcctgagtttgcaaccagttgtagggtttctgttgtgttttttttttttttgaaataaaactataatataaattctcctattaaataaaattattttaagttttagtgtcaaaagtgagatgctgagagtaggtgataatgtatattttacagagtgggggttggcaggatggtgacattgaacatgattgctctctgtctcttttttcagcttatgggtatttatcttctattagtatttgtatcttcagttcattccactttaggaaacagagctgccaattgaaacagaagaagaaaaaaaaaaaagcagcagacaacacactgtagagtcttgcacacacacaagtgcccaggcaaggtgcttggcagaaccgcagagtgggaagagagtaccggcatcgggtttccttgggatcaatttcattaccgtgtacctttcccattgtggtcacgccatttggcagggggagaatgggaggcttggccttctttgtgaggcagtgtgagcagaagaagctgatgccagcatgtcactggttttgaagggatgagcccagacttgatgttttgggattgtccttattttaacctcaaggtctcgcatggtggggcccctgaccaacctacacaagttccctcccacaagtggacatcagtgtcttctctgtgaggcatctggccattcgcactccctggtgtggtcagcctctctcacacaaggaggaacttgggtgaaggctgagtgtgaggcacctgaagtttccctgcggagtcgataaattagcagaaccacatccccatctgttaggccttggtgaggaggccctgggcaaagaagggtctttcgcaaagcgatgtcagagggcggttttgagctttctataagctatagctttgtttatttcacccgttcacttactgtataatttaaaatcatttatgtagctgagacacttctgtatttcaatcatatcatgaacattttattttgctaaatcttgtgtcatgtgtaggctgtaatatgtgtacattgtgtttaagagaaaaatgaaacccacatgccgccattttcctgaatcaaattctgcagtggaatggagaggaaaatacttctaggcaagcagctagactggtgaattgggggaaatagaaggaactagtaactgagactcctccagcctcttccctattggaatcccaatggctcctggagtaggaaaaaagtttaaactacattcatgttcttgttctgtgtcactcggccctgggtagtctaccatttacttcaccccaagtcctgctgcccatccagttgggaagcccatgattttcctaagaatccagggccataggagatacaattccaagttctcgcttcctcctttgggcatctcttctgcctcccaatcaaggaagctccacgctcaggctctcagctctcgggccagtgctctgctctgtccagggtaggtaatactgggagactcctgtcttttaccctcccctcgttccagacctgcctcatggtggcaacatggttcttgaacaattaaagaaacaaatgactttttggaatagccctgtctagggcaaactgtggcccccaggagacactacccttccatgccccagacctctgtcttgcatgtgacaattgacaatctggactaccccaagatggcacccaagtgtttggcttctggctacctaaggttaacatgtcactagagtatttttatgagagacaaacattataaaaatctgatggcaaaagcaaaacaaaatggaaagtaggggaggtggatgtgacaacaacttccaaattggctctttggaggcgagaggaaggggagaacttggagaatagtttttgctttgggggtagaggcttcttagattctcccagcatccgcctttccctttagccagtctgctgtcctgaaacccagaagtgatggagagaaaccaacaagagatctcgaaccctgtctagaaggaatgtatttgttgctaaatttcgtagcactgtttacagttttcctccatgttatttatgaattttatattccgtgaatgtatattgtcttgtaatgttgcataatgttcactttttatagtgtgtcctttattctaaacagtaaagtggttttatttctatcac

SEQ ID NO: 9 - DNA, 476 bp, Homo sapiens
  gene (1) .. (476): JM27; Unigene Hs.95420 (arch.); Hs.441038 (current)
gaagaattcgccaggctctctgctgactcaagttcttcagttcacgatcttctagttgcagcgatgagtgcacgagtgagatcaagatccagaggaagaggagatggtcaggaggctcccgatgtggttgcattcgtggctcccggtgaatctcagcaagaggaaccaccaactgacaatcaggatattgaacctggacaagagagagaaggaacacctccgatcgaagaacgtaaagtagaaggtgattgccaggaaatggatctggaaaagactcggagtgagcgtggagatggctctgatgtaaaagagaagactccacctaatcctaagcatgctaagactaaagaagcaggagatgggcagccataagttaaaaagaagacaagctgaagctacacacatggctgatgtcacattgaaaatgtgactgaaaatttgaaaattctctcaataaagtttgagttttctctgaa

SEQ ID NO: 10 - DNA, 1056 bp, Homo sapiens
  gene (1) .. (1056): COL4A2; Unigene Hs.211933
agatccggacgctggccttgatggggcctcttggtcttcttgggcaaattggcccacttggagctctagggatgctaggccagaagggggagattggactgctaggccctctaggacacgatgggaaaggacctcgcggtaaactaggagacatgggccctcttggtccccaaggccccctaggaaaggatggacctccaggagtgaagggagaaaacgggcacccagggagcccaggagagaagggggaaaaaggggagacaggacaagcaggctcaccggttcctgggctgctagggccagaggggcctcccggacctccggggctccaaggtgttcctggaccaaagggggaagcaggactagacggagcaaaaggagagaaaggcttccagggagaaaaaggagaccgtggtcccctgggactacccggagcttcaggtttggacggcaggcctgggccaccgggtactccaggaccaattggagttccaggcccagcgggaccaaagggcgagaggggcagcaaaggagaccctgggatgacaggaccaacgggagcagctgggcttcctggtttacatggaccacccggggacaagggaaaccggggggagagggggaagaaaggctctagagggcctaaaggggacaagggagaccaaggagcgcctggattagatgccccctgcccattggggcaagatggcttaccagtccaaggctgctggaacaagtgatgcctctaaccttggattggcctgtgtgtgtgtttgtacatagaatatttatttttatacagttttcactttttgaaaatgccagaagtatgatgcatcttacagattattaaaaaagaaagaaaaaccgttgcatattttgtacagaaaatatcaacctcttcccttttgtttacaagatgttttgtataagcctatgtctctaatacattttttgtttggtcgtaatgtctgcatgatatttgtgcacatttattaagtatcgaaggttaataaattgttgtgtcctggtgccaaggggggccagccagaactg

SEQ ID NO: 11 - DNA, 2897 bp, Homo sapiens
  gene (1)..(2897): GATA-6; Unigene Hs.50924 (arch.); Hs.514746 (current)
ggaattccgcgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggtggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggacccttcgagaccccggtgctgcacagcctgcagagccgcgccggagccccgctcccggtgccccggggtcccagtgcagacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcgcgtgccttcatcacggcggcttggattgtcctgtgccaactgtcacaccacaactaccaccttatggcgcagaaacgccgagggtgaacccgtgtgcaatgcttgtggactctacatgaaactccatggggtgcccagaccacttgctatgaaaaaagagggaattcaaaccaggaaacgaaaacctaagaacataaataaatcaaagacttgctctggtaatagcaataattccattcccatgactccaacttccacctcttctaactcagatgattgcagcaaaaatacttcccccacaacacaacctacagcctcaggggcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggcctgagcccacgccgccaggaggcagggagggctccgccgcgggcctcactccactcgtgtctgcttttgtgcagcggtccagacagtggcgactgcgctgacagaacgtgattctcgtgcctttattttgaaagagatgtttttcccaagaggcttgctgaaagagtgagagaagatggaagggaagggccagtgcaactgggcgcttgggccactccagccagcccgcctccggggcggaccctgctccacttccagaagccaggactaggacctgggccttgcctgctatggaatattgagagagattttttaaaaaagattttgcattttgtccaaaatcatgtgcttcttctgatcaattttggttgttccagaatttcttcataccttttccacatccagatttcatgtgcgttcatggagaagatcacttgaggccatttggtacacatctctggaggctgagtcggttcatgaggtctcttatcaaaaatattactcagtttgcaagactgcattgtaactttaacatacactgtgactgacgtttctcaaagttcatattgtgtggctgatctgaagtcagtcggaatttgtaaacagggtagcaaacaagatatttttcttccatgtatacaataatttttttaaaaagtgcaatttgcgttgcagcaatcagtgttaaatcatttgcataagatttaacagcattttttataatgaatgtaaacattttaacttaatggtacttaaaataatttaaaagaaaaatgttaacttagacattcttatgcttcttttacaactacatcccattttatatttccaattgttaaagaaaaatatttcaagaacaaatcttctctcaggaaaattgcctttctctatttgttaagaatttttatacaagaacaccaatataccccctttattttactgtggaatatgtgctggaaaaattgcaacaacactttactacctaacggatagcatttgtaaatactctaggtatctgtaaacactctgatgaagtctgtatagtgtgactaacccacaggcaggttggtttacattaattttttttttgaatgggatgtcctatggaaacctatttcaccagagttttaaaaataaaaagggtattgttttgtcttctgtaaaaaaaaaaaaaaaaaaaaaaaaaaaaaaaaaaaaaaaaaaa

SEQ ID NO: 12 - DNA, 3541 bp, Homo sapiens
  gene (1)..(3541): CXCL12; Unigene Hs.237356 (arch.); Hs.522891 (curr.)
cgcggccgcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgagagggtcagacgcctgaggaacccttacagtaggagtccagctctgaaaccagtgttagggaagggcctgccacagcctcccctgccagggcagggccccaggcattgccaagggctttgttttggacactttgccatattttcaccatttgattatgtagcaaaatacatgacatttatttttcatttagtttgattattcagtgtcactggcgacacgtagcagcttagactaaggccattattgtacttgccttattagagtgtctttccacggagccactcctctgactcagggctcctgggttttggattctctgagctgtgcaggtggggagactgggctgagggagcctggccccatggtcagccctagggtggagagccaccaagagggacgcctgggggtgtcaggaccagtcaacctgggcaaagcctagtgaaggcttctctctgtgggatgggatggtggagggccacatgggaggttcacccccttctccatccacatggtgagccgggtctgcctcttctgggagggcagcagggctaccctgagctgaggcagcagtgtgaggccagggcagagtgagacccagccctcatcccgagcacctccacatcctccacgttctgctcatcattctctgtctcatccatcatcatgtgtgtccacgactgtctccatggccccgcaaaaggactctcaggaccaaagctttcatgtaaactgtgcaccaagcaggaaatgaaaatgtcttgtgttacctgaaaacactgtgcacatctgtgtcttgtttggaatattgtccattgtccaatcctatgtttttggtcaaagccagcgtcctcctctgtgaccaatgtcttgatgcatgcactgttccccctgtgcagccgctgagcgaggagatgctccntgggccctttgagtgcagtcctgatcagagccgtggtcctttggggtgaactaccttggttcccccactgatcacaaaaacatggtgggtccatgggcagagcccaagggaattcggtgtgcaccagggttgaccccagaggattgctgccccatcagtgctccctcacatgtcagtaccttcaaactagggccaagcccagcactgcttgaggaaaacaagcattcacaacttgttttnggtttttaaaacccagtccacaaaataaccaatcctggacatgaagattctttcccaattcacatctaacctcatcttcttcaccatttggcaatgccatcatctcctgccttcctcctgggccctctctgctctgcgtgtcacctgtgcttcgggcccttcccacaggacatttctctaagagaacaatgtgctatgtgaagagtaagtcaacctgcctgacatttggagtgttccccttccactgagggcagtcgatagagctgtattaagccacttaaaatgtttgtcactttgccaaggcaagcacttgtgggnnttgnttgttntcantcagtcttncgaatactttttccccttgataaagactccagttaaaanaaattttaatgaagaaagtggaaacaaggaagtcaaagcaaggaaactatgtaacatgtaggaagtaggaagtaaattatagtgatgtaatcttgaattgtaactgttcttgaatttaataatctgtagggtaattagtaacatgtgttaagtattttcataagtatttcaaattggagcttcatggcagaaggcaaacccatcancaaaaattgtcccttaaacaaaaattaaaatcctcaatccagctatgttatattgaaaaaatagagcctgagggatctttactagttataaagatacagaactctttcnaaaccttttgaaattaacctctcactataccagtataattgagttttcagtggggcagtcattatccaggtaatccaagatattttaaaatctgtcacgtagaacttggatgtacctgcccccaatccatgaaccaagaccattgaattcttggttgaggaaacaaacatgaccctaaatcttgactacagtcaggaaaggaatcatttctatttctcctccatgggagaaaatagataagagtagaaactgcagggnaaaattatttgnataacaattcctctactaacaatcagctccttcctggagactgcccagctaaagcaatatgcatttaaatacagtcttccatttgnaagggaaaagtctcttgtaatccgaatctctttttggtttcgaactgctagtcaagtgcgtccacgagctgtttactagggatccctcatctgtccctccgggacctggtgctgcctctacctgacactcccttgggctccctgtaacctcttcagaggncctcgctgccagctctgtntcaggacccagaggaaggggncagaggctcgttgactggctgtgtgttgggattgagtctgtgccacgtgtttgtgctgtggtgtgtcccctctgtccaggcactgagataccagcgaggaggctccagagggcgctctgcttgttattagagattacctcctgagaaaaaaggttccgcttggagcagaggggctgaatagcagaaggttgcacctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgnaacctcgctggactctcagtctctgagcagtgaatgattcagtgttaaatgtgatgaatactgtattttgtattgtttcaattgcatctcccagataatgtgaaaatggtccaggagaaggncaattcctatacgcagngtgctttaaaaaataaataagaaacaactctttgagaaacaacaatttctactttgaagtcataccaatgaaaaaatgtatatgcacttataattttcctaataaagttctgtactcaaatgtaaaaaaaaaaaaaaaaaaaa

SEQ ID NO: 13 - DNA, 1356 bp, Homo sapiens
  gene (1)..(1356): IGF-2; Unigene Hs.251664(arch.); Hs.272259 (curr.)
ttctcccgcaaccttcccttcgctccctcccgtcccccccagctcctagcctccgactccctccccccctcacgcccgccctctcgccttcgccgaaccaaagtggattaattacacgctttctgtttctctccgtgctgttctctcccgctgtgcgcctgcccgcctctcgctgtcctctctccccctcgccctctcttcggcccccccctttcacgttcactctgtctctcccactatctctgcccccctctatccttgatacaacagctgacctcatttcccgataccttttcccccccgaaaagtacaacatctggcccgccccagcccgaagacagcccgtcctccctggacaatcagacgaattctccccccccccccaaaaaaaaaagccatccccccgctctgccccgtcgcacattcggcccccgcgactcggccagagcggcgctggcagaggagtgtccggcaggagggccaacgcccgctgttcggtttgcgacacgcagcagggaggtgggcggcagcgtcgccggcttccagacaccaatgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagtgagcaaaactgccgcaagtctgcagcccggcgccaccatcctgcagcctcctcctgaccacggacgtttccatcaggttccatcccgaaaatctctcggttccacgtccccctggggcttctcctgacccagtccccgtgccccgcctccccgaaacaggctactctcctcggccccctccatcgggctgaggaagcacagcagcatcttcaaacatgtacaaaatcgattggctttaaacacccttcacataccctcccccc

SEQ ID NO: 14 - DNA, 2574 bp, Homo sapiens
  gene (1)..(2574): TGF-beta3; Unigene Hs.2025 (arch.); Hs.592317 (curr.)
cctgtttagacacatggacaacaatcccagcgctacaaggcacacagtccgcttcttcgtcctcagggttgccagcgcttcctggaagtcctgaagctctcgcagtgcagtgagttcatgcaccttcttgccaagcctcagtctttgggatctggggaggccgcctggttttcctccctccttctgcacgtctgctggggtctcttcctctccaggccttgccgtccccctggcctctcttcccagctcacacatgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctgagaccccacgtgcgacagagagaggggagagagaaccaccactgcctgactgcccgctcctcgggaaacacacaagcaacaaacctcactgagaggcctggagcccacaaccttcggctccgggcaaatggctgagatggaggtttccttttggaacatttctttcttgctggctctgagaatcacggtggtaaagaaagtgtgggtttggttagaggaaggctgaactcttcagaacacacagactttctgtgacgcagacagaggggatggggatagaggaaagggatggtaagttgagatgttgtgtggcaatgggatttgggctaccctaaagggagaaggaagggcagagaatggctgggtcagggccagactggaagacacttcagatctgaggttggatttgctcattgctgtaccacatctgctctagggaatctggattatgttatacaaggcaagcatttttttttttaaagacaggttacgaagacaaagtcccagaattgtatctcatactgtctgggattaagggcaaatctattacttttgcaaactgtcctctacatcaattaacatcgtgggtcactacagggagaaaatccaggtcatgcagttcctggcccatcaactgtattgggccttttggatatgctgaacgcagaagaaagggtggaaatcaaccctctcctgtctgccctctgggtccctcctctcacctctccctcgatcatatttccccttggacacttggttagacgccttccaggtcaggatgcacatttctggattgtggttccatgcagccttggggcattatgggtcttcccccacttcccctccaagaccctgtgttcatttggtgttcctggaagcaggtgctacaacatgtgaggcattcggggaagctgcacatgtgccacacagtgacttggccccagacgcatagactgaggtataaagacaagtatgaatattactctcaaaatctttgtataaataaatatttttggggcatcctggatgatttcatcttctggaatattgtttctagaacagtaaaagccttattctaaggtg

SEQ ID NO: 15 - DNA, 647 bp, Homo sapiens
  gene (1)..(647): PTGDS; Unigene Hs.8272 (arch.); Hs.446429 (curr.)
gctcctcctgcacacctccctcgctctcccacaccactgcaccaggccccggacacccgctctgctgcaggagaatggctactcatcacacgctgtggatgggactggccctgctgggggtgctgggcgacctgcaggcagcaccggaggcccaggtctccgtgcagcccaacttccagcaggacaagttcctggggcgctggttcagcgcgggcctcgcctccaactcgagctggctccgggagaagaaggcggcgttgtccatgtgcaagtctgtggtggcccctgccacggatggtggcctcaacctgacctccaccttcctcaggaaaaaccagtgtgagacccgaaccatgctgctgcagcccgcggggtccctcggctcctacagctaccggagtccccactggggcagcacctactccgtgtcagtggtggagaccgactacgaccagtacgcgctgctgtacagccagggcagcaagggccctggcgaggacttccgcatggccaccctctacagccgaacccagacccccagggctgagttaaaggagaaattcaccgccttctgcaaggcccagggcttcacagaggataccattgtcttcctgccccaaaccgataagtgcatgacggaacaatag

(19) United States
(12) Patent Application Publication
(10) Pub. No.: US 2014/0018249 A1
(43) Pub. Date: Jan. 16, 2014

(54) BIOMARKERS FOR SCREENING, PREDICTING, AND MONITORING BENIGN PROSTATE HYPERPLASIA

(71) Applicant: Isabelle Guyon, Berkeley, CA (US)

(72) Inventor: Isabelle Guyon, Berkeley, CA (US)

(73) Assignee: Health Discovery Corporation, Savannah, GA (US)

(21) Appl. No.: 13/418,291

(22) Filed: Mar. 12, 2012

Related U.S. Application Data
(62) Division of application No. 11/829,039, filed on Jul. 26, 2007, now abandoned.
(60) Provisional application No. 60/833,644, filed on Jul. 26, 2006.

(51) Int. Cl. C12Q 1/68

(52) U.S. Cl. CPC C12Q 1/6886
USPC 506/9

(57) ABSTRACT

Gene expression data are analyzed using learning machines such as support vector machines (SVM) and ridge regression classifiers to rank genes according to their ability to distinguish between BPH (benign prostatic hyperplasia) and all other conditions. Results are provided showing the correlation of results obtained using data from two independent studies that took place at different times using different microarrays. Genes are ranked according to area-under-the-curve, false discovery rate and fold change.

### RELATED APPLICATIONS

The present application claims priority to U.S. application Ser. No. 11/829,039 filed on Jul. 26, 2007, which claims priority to U.S. Provisional Application No. 60/833,644, filed Jul. 26, 2006.

This application is related to, but does not claim the priority of U.S. application Ser. No. 11/274,931, filed Nov. 14, 2005 which claims priority to each of U.S. Provisional Applications No. 60/627,626, filed Nov. 12, 2004, and No. 60/651,340, filed Feb. 9, 2005.

This application is also related to, but does not claim the priority, of U.S. application Ser. No. 10/057,849, now issued as U.S. Pat. No. 7,117,188, which claims priority to each of U.S. Provisional Applications No. 60/263,696, filed Jan. 24, 2001, No. 60/298,757, filed Jun. 15, 2001, and No. 60/275,760, filed Mar. 14, 2001, and is a continuation-in-part of U.S. patent application Ser. No. 09/633,410, filed Aug. 7, 2000, now issued as U.S. Pat. No. 6,882,990, which claims priority to each of U.S. Provisional Applications No. 60/161,806, filed Oct. 27, 1999, No. 60/168,703, filed Dec. 2, 1999, No. 60/184,596, filed Feb. 24, 2000, No. 60/191,219, filed Mar. 22, 2000, and No. 60/207,026, filed May 25, 2000, and is a continuation-in-part of U.S. patent application Ser. No. 09/578,011, filed May 24, 2000, now issued as U.S. Pat. No. 6,658,395, which claims priority to U.S. Provisional Application No. 60/135,715, filed May 25, 1999, and is a continuation-in-part of application Ser. No. 09/568,301, filed May 9, 2000, now issued as U.S. Pat. No. 6,427,141, which is a continuation of application Ser. No. 09/303,387, filed May 1, 1999, now issued as U.S. Pat. No. 6,128,608, which claims priority to U.S. Provisional Application No. 60/083,961, filed May 1, 1998. This application is related to co-pending application Ser. No. 09/633,615, now abandoned, Ser. No. 09/633,616, now issued as U.S. Pat. No. 6,760,715, Ser. No. 09/633,627, now issued as U.S. Pat. No. 6,714,925, and Ser. No. 09/633,850, now issued as U.S. Pat. No. 6,789,069, all filed Aug. 7, 2000, which are also continuations-in-part of application Ser. No. 09/578,011. Each of the above cited applications and patents are incorporated herein by reference.

### FIELD OF THE INVENTION

The present invention relates to the use of learning machines to identify relevant patterns in datasets containing large quantities of gene expression data, and more particularly to biomarkers so identified for use in screening, predicting, and monitoring benign prostate hyperplasia.

### BACKGROUND OF THE INVENTION

Knowledge discovery is the most desirable end product of data collection. Recent advancements in database technology have lead to an explosive growth in systems and methods for generating, collecting and storing vast amounts of data. While database technology enables efficient collection and storage of large data sets, the challenge of facilitating human comprehension of the information in this data is growing ever more difficult. With many existing techniques the problem has become unapproachable. In particular, methods are needed for identifying patterns in biological systems as reflected in gene expression data.

A large fraction of men (20%) in the U.S. are diagnosed with prostate cancer during their lifetime, with nearly 300,000 men diagnosed annually, a rate second only to skin cancer. However, only 3% of those die of the disease. About 70% of all diagnosed prostate cancers are found in men aged 65 years and older. Many prostate cancer patients have undergone aggressive treatments that can have life-altering side effects such as incontinence and sexual dysfunction. It is believed that a large fraction of the cancers are over-treated. Currently, most early prostate cancer identification is done using prostate-specific antigen (PSA) screening, but few indicators currently distinguish between progressive prostate tumors that may metastasize and escape local treatment and indolent cancers of benign prostate hyperplasia (BPH). Further, some studies have shown that PSA is a poor predictor of cancer, instead tending to predict BPH, which requires no treatment.

There is an urgent need for new biomarkers for distinguishing between normal, benign and malignant prostate tissue and for predicting the size and malignancy of prostate cancer. Blood serum biomarkers would be particularly desirable for screening prior to biopsy, however, evaluation of gene expression microarrays from biopsied prostate tissue is also useful.

### SUMMARY OF THE INVENTION

Gene expression data are analyzed using learning machines such as support vector machines (SVM) and ridge regression classifiers to rank genes according to their ability to separate BPH (benign prostatic hyperplasia) from other prostate conditions including cancer and normal. Small groups of genes are identified that provide sensitivities and selectivities of better than 90% for separating BPH from other prostate conditions.

A preferred embodiment comprises methods and systems for detecting genes involved with prostate cancer and determination of methods and compositions for treatment of prostate cancer. In one embodiment, to improve the statistical significance of the results, supervised learning techniques can analyze data obtained from a number of different sources using different microarrays, such as the Affymetrix U95 and U133A chip sets.

### BRIEF DESCRIPTION OF THE DRAWINGS

FIG. 1 is a functional block diagram illustrating an exemplary operating environment for an embodiment of the present invention.

FIG. 2 is a plot showing the results based on LCM data preparation for prostate cancer analysis.

FIG. 3 is a plot graphically comparing SVM-RFE of the present invention with leave-one-out classifier for prostate cancer.

FIG. 4 graphically compares the Golub and SVM methods for prostate cancer.

FIGS. 5a-5s combined are two tables showing the top 200 genes for separating BPH from all other tissues that were identified in each of the 2001 study and the 2003 study.

FIG. 6 is a diagram of a hierarchical decision tree for BPH, G3&G4, Dysplasia, and Normal cells.

FIG. 7 is a graph of ROC curves of the top most discriminative genes for distinguishing BPH from all others.

FIG. 8 is a plot of AUC for varying numbers of discriminative BPH genes.

### DETAILED DESCRIPTION OF THE PREFERRED EMBODIMENT

The present invention utilizes learning machine techniques, including support vector machines and ridge regression, to discover knowledge from gene expression data obtained by measuring hybridization intensity of gene and gene fragment probes on microarrays. The knowledge so discovered can be used for diagnosing and prognosing changes in biological systems, such as diseases. Preferred embodiments comprise identification of genes that will distinguish between different types of prostate disorders, such as benign prostate hyperplasy and cancer, and normal, and use of such information for decisions on treatment of patients with prostate disorders.

The problem of selection of a small amount of data from a large data source, such as a gene subset from a microarray, is particularly solved using the methods described herein. Preferred methods described herein use support vector machine (SVM) methods based on recursive feature elimination (RFE). In examining gene expression data to find determinative genes, these methods eliminate gene redundancy automatically and yield better and more compact gene subsets.

The data is input into computer system, preferably a SVM-RFE. The SVM-RFE is run one or more times to generate the best features selections, which can be displayed in an observation graph. The SVM may use any algorithm and the data may be preprocessed and postprocessed if needed. Preferably, a server contains a first observation graph that organizes the results of the SVM activity and selection of features.

The information generated by the SVM may be examined by outside experts, computer databases, or other complementary information sources. For example, if the resulting feature selection information is about selected genes, biologists or experts or computer databases may provide complementary information about the selected genes, for example, from medical and scientific literature. Using all the data available, the genes are given objective or subjective grades. Gene interactions may also be recorded.

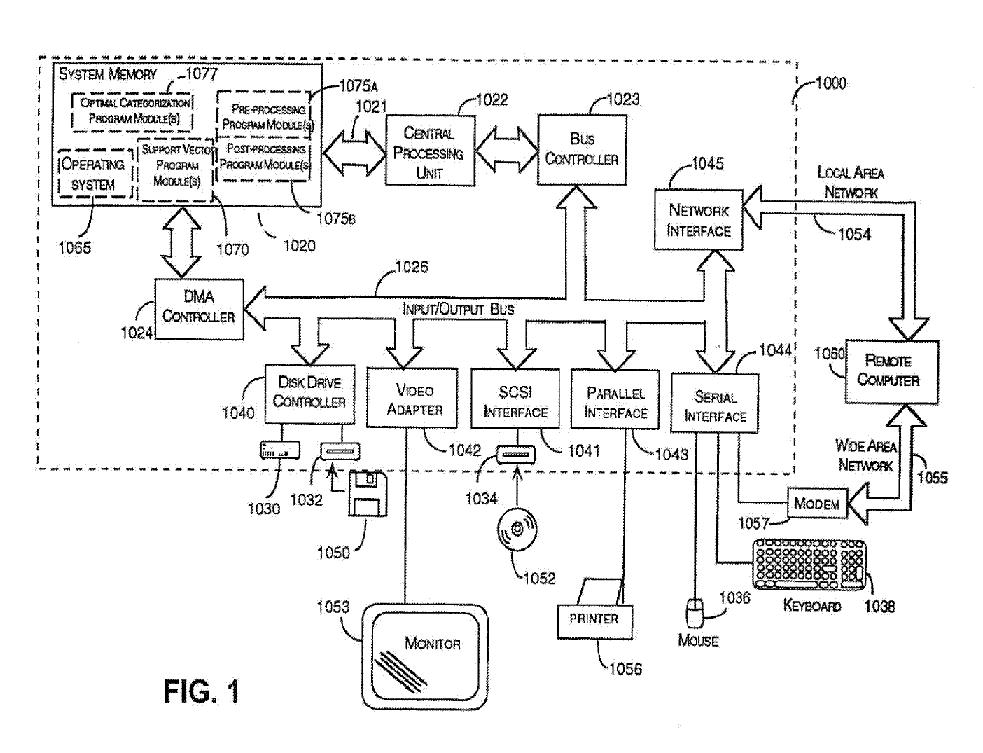

FIG. 1 and the following discussion are intended to provide a brief and general description of a suitable computing environment for implementing biological data analysis according to the present invention. Although the system shown in FIG. 1 is a conventional personal computer 1000, those skilled in the art will recognize that the invention also may be implemented using other types of computer system configurations. The computer 1000 includes a central processing unit 1022, a system memory 1020, and an Input/Output (“I/O”) bus 1026. A system bus 1021 couples the central processing unit 1022 to the system memory 1020. A bus controller 1023 controls the flow of data on the I/O bus 1026 and between the central processing unit 1022 and a variety of internal and external I/O devices. The I/O devices connected to the I/O bus 1026 may have direct access to the system memory 1020 using a Direct Memory Access (“DMA”) controller 1024.

The I/O devices are connected to the I/O bus 1026 via a set of device interfaces. The device interfaces may include both hardware components and software components. For instance, a hard disk drive 1030 and a floppy disk drive 1032 for reading or writing removable media 1050 may be connected to the I/O bus 1026 through disk drive controllers 1040. An optical disk drive 1034 for reading or writing optical media 1052 may be connected to the I/O bus 1026 using a Small Computer System Interface (“SCSI”) 1041. Alternatively, an IDE (Integrated Drive Electronics, i.e., a hard disk drive interface for PCs), ATAPI (ATtAchment Packet Interface, i.e., CD-ROM and tape drive interface), or EIDE (Enhanced IDE) interface may be associated with an optical drive such as may be the case with a CD-ROM drive. The drives and their associated computer-readable media provide nonvolatile storage for the computer 1000. In addition to the computer-readable media described above, other types of computer-readable media may also be used, such as ZIP drives, or the like.

A display device 1053, such as a monitor, is connected to the I/O bus 1026 via another interface, such as a video adapter 1042. A parallel interface 1043 connects synchronous peripheral devices, such as a laser printer 1056, to the I/O bus 1026. A serial interface 1044 connects communication devices to the I/O bus 1026. A user may enter commands and information into the computer 1000 via the serial interface 1044 or by using an input device, such as a keyboard 1038, a mouse 1036 or a modem 1057. Other peripheral devices (not shown) may also be connected to the computer 1000, such as audio input/output devices or image capture devices.

A number of program modules may be stored on the drives and in the system memory 1020. The system memory 1020 can include both Random Access Memory (“RAM”) and Read Only Memory (“ROM”). The program modules control how the computer 1000 functions and interacts with the user, with I/O devices or with other computers. Program modules include routines, operating systems 1065, application programs, data structures, and other software or firmware components. In an illustrative embodiment, the learning machine may comprise one or more pre-processing program modules 1075A, one or more post-processing program modules 1075B, and/or one or more optimal categorization program modules 1077 and one or more SVM program modules 1070 stored on the drives or in the system memory 1020 of the computer 1000. Specifically, pre-processing program modules 1075A, post-processing program modules 1075B, together with the SVM program modules 1070 may comprise computer-executable instructions for pre-processing data and post-processing output from a learning machine and implementing the learning algorithm. Furthermore, optimal categorization program modules 1077 may comprise computer-executable instructions for optimally categorizing a data set.

The computer 1000 may operate in a networked environment using logical connections to one or more remote computers, such as remote computer 1060. The remote computer 1060 may be a server, a router, a peer to peer device or other common network node, and typically includes many or all of the elements described in connection with the computer 1000. In a networked environment, program modules and data may be stored on the remote computer 1060. Appropriate logical connections include a local area network (“LAN”) and a wide area network (“WAN”). In a LAN environment, a network interface, such as an Ethernet adapter card, can be used to connect the computer to the remote computer. In a WAN environment, the computer may use a telecommunications device, such as a modem, to establish a connection. It will be appreciated that the network connections shown are illustrative and other devices of establishing a communications link between the computers may be used.

A preferred selection browser is preferably a graphical user interface that would assist final users in using the generated information. For example, in the examples herein, the selection browser is a gene selection browser that assists the final user is selection of potential drug targets from the genes identified by the SVM RFE. The inputs are the observation graph, which is an output of a statistical analysis package and any complementary knowledge base information, preferably in a graph or ranked form. For example, such complementary information for gene selection may include knowledge about the genes, functions, derived proteins, measurement assays, isolation techniques, etc. The user interface preferably allows for visual exploration of the graphs and the product of the two graphs to identify promising targets. The browser does not generally require intensive computations and if needed, can be run on other computer means. The graph generated by the server can be precomputed, prior to access by the browser, or is generated in situ and functions by expanding the graph at points of interest.

In a preferred embodiment, the server is a statistical analysis package, and in the gene feature selection, a gene selection server. For example, inputs are patterns of gene expression, from sources such as DNA microarrays or other data sources. Outputs are an observation graph that organizes the results of one or more runs of SVM RFE. It is optimum to have the selection server run the computationally expensive operations.

A preferred method of the server is to expand the information acquired by the SVM. The server can use any SVM results, and is not limited to SVM RFE selection methods. As an example, the method is directed to gene selection, though any data can be treated by the server. Using SVM RFE for gene selection, gene redundancy is eliminated, but it is informative to know about discriminant genes that are correlated with the genes selected. For a given number N of genes, only one combination is retained by SVM-RFE. In actuality, there are many combinations of N different genes that provide similar results.

A combinatorial search is a method allowing selection of many alternative combinations of N genes, but this method is prone to overfitting the data. SVM-RFE does not overfit the data. SVM-RFE is combined with supervised clustering to provide lists of alternative genes that are correlated with the optimum selected genes. Mere substitution of one gene by another correlated gene yields substantial classification performance degradation.

The examples included herein show preferred methods for determining the genes that are most correlated to the presence of cancer or can be used to predict cancer occurrence in an individual. There is no limitation to the source of the data and the data can be combinations of measurable criteria, such as genes, proteins or clinical tests, that are capable of being used to differentiate between normal conditions and changes in conditions in biological systems.

In the following examples, preferred numbers of genes were determined that result from separation of the data that discriminate. These numbers are not limiting to the methods of the present invention. Preferably, the preferred optimum number of genes is a range of approximately from 1 to 500, more preferably, the range is from 10 to 250, from 1 to 50, even more preferably the range is from 1 to 32, still more preferably the range is from 1 to 21 and most preferably, from 1 to 10. The preferred optimum number of genes can be affected by the quality and quantity of the original data and thus can be determined for each application by those skilled in the art.

Once the determinative genes are found by the learning machines of the present invention, methods and compositions for treatments of the biological changes in the organisms can be employed. For example, for the treatment of cancer, therapeutic agents can be administered to antagonize or agonize, enhance or inhibit activities, presence, or synthesis of the gene products. Therapeutic agents and methods include, but are not limited to, gene therapies such as sense or antisense polynucleotides, DNA or RNA analogs, pharmaceutical agents, plasmaphoresis, antiangiogenics, and derivatives, analogs and metabolic products of such agents.

Such agents may be administered via parenteral or noninvasive routes. Many active agents are administered through parenteral routes of administration, intravenous, intramuscular, subcutaneous, intraperitoneal, intraspinal, intrathecal, intracerebroventricular, intraarterial and other routes of injection. Noninvasive routes for drug delivery include oral, nasal, pulmonary, rectal, buccal, vaginal, transdermal and occular routes.

The following examples illustrate the results of using SVMs and other learning machines to identify genes associated with disorders of the prostate. Such genes may be used for diagnosis, treatment, in terms of identifying appropriate therapeutic agents, and for monitoring the progress of treatment.

### Example 1

### Isolation of Genes Involved with Prostate Cancer

Using the methods disclosed herein, genes associated with prostate cancer were isolated. Various methods of treating and analyzing the cells, including SVM, were utilized to determine the most reliable method for analysis.

Tissues were obtained from patients that had cancer and had undergone prostatectomy. The tissues were processed according to a standard protocol of Affymetrix and gene expression values from 7129 probes on the Affymetrix HuGeneFL GeneChip® were recorded for 67 tissues from 26 patients.

Specialists of prostate histology recognize at least three different zones in the prostate: the peripheral zone (PZ), the central zone (CZ), and the transition zone (TZ). In this study, tissues from all three zones are analyzed because previous findings have demonstrated that the zonal origin of the tissue is an important factor influencing the genetic profiling. Most prostate cancers originate in the PZ. Cancers originating in the PZ have worse prognosis than those originating in the TZ. Contemporary biopsy strategies concentrate on the PZ and largely ignore cancer in the TZ. Benign prostate hyperplasia (BPH) is found only in the TZ. BPH is a suitable control that may be used to compare cancer tissues in genetic profiling experiments. BPH is also convenient to use as control because it is abundant and easily dissected. However, controls coming from normal tissues microdissected with lasers in the CZ and PZ can also provide important complementary controls. The gene expression profile differences have been found to be larger between PZ-G4-G5 cancer and CZ-normal used as control, compared to PZ-normal used as control. A possible explanation comes from the fact that is presence of cancer, even normal adjacent tissues have undergone DNA changes (Malins et al, 2003-2004). Table 1 gives zone properties.

TABLE 1

| Zone | Properties |
|---|---|
| PZ | From apex posterior to base, surrounds transition and central zones. |
| | Largest zone (70% in young men). |
| | Largest number cancers (60-80%). |
| | Dysplasia and atrophy common in older men. |
| CZ | Surrounds transition zone to angle of urethra to bladder base. |
| | Second largest zone (25% in young men to 30% at 40 year old). |
| | 50% of PSA secreting epithelium. |
| | 5-20% of cancers. |
| TZ | Two pear shaped lobes surrounding the proximal urethra. |
| | Smallest zone in young men (less than 5%). |
| | Gives rise to BPH in older men. May expand to the bulk of the |
| | gland. |
| | 10-18% of cancers. |
| | Better cancer prognosis than PZ cancer. |

Classification of cancer determines appropriate treatment and helps determine the prognosis. Cancer develops progressively from an alteration in a cell's genetic structure due to mutations, to cells with uncontrolled growth patterns. Classification is made according to the site of origin, histology (or cell analysis; called grading), and the extent of the disease (called staging).

Prostate cancer specialists classify cancer tissues according to grades, called Gleason grades, which are correlated with the malignancy of the diseases. The larger the grade, the poorer the prognosis (chances of survival). In this study, tissues of grade 3 and above are used. Grades 1 and 2 are more difficult to characterize with biopsies and not very malignant. Grades 4 and 5 are not very differentiated and correspond to the most malignant cancers: for every 10% increase in the percent of grade 4/5 tissue found, there is a concomitant increase in post radical prostatectomy failure rate. Each grade is defined in Table 2.

TABLE 2

| Grade | Description |
|---|---|
| 1 | Single, separate, uniform, round glands closely packed with a |
| | definite rounded edge limiting the area of the tumor. Separation of |
| | glands at the periphery from the main collection by more than one |
| | gland diameter indicates a component of at least grade 2. |
| | Uncommon pattern except in the TZ. Almost never seen in needle |
| | biopsies. |
| 2 | Like grade 1 but more variability in gland shape and more stroma |
| | separating glands. Occasional glands show angulated or distorted |
| | contours. More common in TZ than PZ. Pathologists don't diagnose |
| | Gleason grades 1 or 2 on prostate needle biopsies since they are |
| | uncommon in the PZ, there is inter-pathologist variability and |
| | poor correlation with radical prostatectomy. |
| 3 | G3 is the most commonly seen pattern. Variation in size, shape |
| | (may be angulated or compressed), and spacing of glands (may be |
| | separated by >1 gland diameter). Many small glands have occluded |
| | or abortive lumens (hollow areas). There is no evidence of glandular |
| | fusion. The malignant glands infiltrate between benign glands. |
| 4 | The glands are fused and there is no intervening stroma. |
| 5 | Tumor cells are arranged in solid sheets with no attempts at gland |
| | formation. The presence of Gleason grade 5 and high percent |
| | carcinoma at prostatectomy predicts early death. |

Staging is the classification of the extent of the disease. There are several types of staging methods. The tumor, node, metastases (TNM) system classifies cancer by tumor size (T), the degree of regional spread or lymph node involvement (N), and distant metastasis (M). The stage is determined by the size and location of the cancer, whether it has invaded the prostatic capsule or seminal vesicle, and whether it has metastasized. For staging, MRI is preferred to CT because it permits more accurate T staging. Both techniques can be used in N staging, and they have equivalent accuracy. Bone scintigraphy is used in M staging.

The grade and the stage correlate well with each other and with the prognosis. Adenocarcinomas of the prostate are given two grade based on the most common and second most common architectural patterns. These two grades are added to get a final score of 2 to 10. Cancers with a Gleason score of <6 are generally low grade and not aggressive.

The samples collected included tissues from the Peripheral Zone (PZ); Central Zone (CZ) and Transition Zone (TZ). Each sample potentially consisted of four different cell types: Stomal cells (from the supporting tissue of the prostate, not participating in its function); Normal organ cells; Benign prostatic hyperplasia cells (BPH); Dysplasia cells (cancer precursor stage) and Cancer cells (of various grades indicating the stage of the cancer). The distribution of the samples in Table 3 reflects the difficulty of obtaining certain types of tissues:

TABLE 3

| | | | | | Cancer | Cancer | |
|---|---|---|---|---|---|---|---|
| | Stroma | Normal | BPH | Dysplasia | G3 | G4 | G3 + G4 |
| PZ | 1 | 5 | | 3 | 10 | 24 | 3 |
| CZ | | 3 | | | | | |
| TZ | | | 18 | | | | |

Benign Prostate Hyperplasia (BPH), also called nodular prostatic hyperplasia, occurs frequently in aging men. By the eighth decade, over 90% of males will have prostatic hyperplasia. However, in only a minority of cases (about 10%) will this hyperplasia be symptomatic and severe enough to require surgical or medical therapy. BPH is not a precursor to carcinoma.

It has been argued in the medical literature that TZ BPH could serve as a good reference for PZ cancer. The highest grade cancer (G4) is the most malignant. Part of these experiments are therefore directed towards the separation of BPH vs. G4.

Some of the cells were prepared using laser confocal microscopy (LCM which was used to eliminate as much of the supporting stromal cells as possible and provides purer samples.

Gene expression was assessed from the presence of mRNA in the cells. The mRNA is converted into cDNA and amplified, to obtain a sufficient quantity. Depending on the amount of mRNA that can be extracted from the sample, one or two amplifications may be necessary. The amplification process may distort the gene expression pattern. In the data set under study, either 1 or 2 amplifications were used. LCM data always required 2 amplifications. The treatment of the samples is detailed in Table 4.

TABLE 4

| | 1 amplification | 2 amplifications |
|---|---|---|
| No LCM | 33 | 14 |
| LCM | | 20 |

The end result of data extraction is a vector of 7129 gene expression coefficients.

Gene expression measurements require calibration. A probe cell (a square on the array) contains many replicates of the same oligonucleotide (probe) that is a 25 bases long sequence of DNA. Each “perfect match” (PM) probe is designed to complement a reference sequence (piece of gene). It is associated with a “mismatch” (MM) probe that is identical except for a single base difference in the central position. The chip may contain replicates of the same PM probe at different positions and several MM probes for the same PM probe corresponding to the substitution of one of the four bases. This ensemble of probes is referred to as a probe set. The gene expression is calculated as:

Average Difference=1/pair num Σ_{probe set} (PM−MM)

If the magnitude of the probe pair values is not contrasted enough, the probe pair is considered dubious. Thresholds are set to accept or reject probe pairs. Affymetrix considers samples with 40% or over acceptable probe pairs of good quality. Lower quality samples can also be effectively used with the SVM techniques.

A simple “whitening” was performed as pre-processing, so that after pre-processing, the data matrix resembles “white noise”. In the original data matrix, a line of the matrix represented the expression values of 7129 genes for a given sample (corresponding to a particular combination of patient/tissue/preparation method). A column of the matrix represented the expression values of a given gene across the 67 samples. Without normalization, neither the lines nor the columns can be compared. There are obvious offset and scaling problems. The samples were pre-processed to: normalize matrix columns; normalize matrix lines; and normalize columns again. Normalization consists of subtracting the mean and dividing by the standard deviation. A further normalization step was taken when the samples are split into a training set and a test set.

The mean and variance column-wise was computed for the training samples only. All samples (training and test samples) were then normalized by subtracting that mean and dividing by the standard deviation.

Samples were evaluated to determine whether LCM data preparation yields more informative data than unfiltered tissue samples and whether arrays of lower quality contain useful information when processed using the SVM technique.

Two data sets were prepared, one for a given data preparation method (subset 1) and one for a reference method (subset 2). For example, method 1=LCM and method 2=unfiltered samples. Golub's linear classifiers were then trained to distinguish between cancer and normal cases using subset 1 and another classifier using subset 2. The classifiers were then tested on the subset on which they had not been trained (classifier 1 with subset 2 and classifier 2 with subset 1).

If classifier 1 performs better on subset 2 than classifier 2 on subset 1, it means that subset 1 contains more information to do the separation cancer vs. normal than subset 2.

The input to the classifier is a vector of n “features” that are gene expression coefficients coming from one microarray experiment. The two classes are identified with the symbols (+) and (−) with “normal” or reference samples belong to class (+) and cancer tissues to class (−). A training set of a number of patterns {x₁, x₂, . . . xₖ, . . . xₗ} with known class labels {y₁, y₂, . . . yₖ, . . . yₗ}, yₖε{−1,+1}, is given. The training samples are used to build a decision function (or discriminant function) D(x), that is a scalar function of an input pattern x. New samples are classified according to the sign of the decision function:

D(x)0 [symbol] xεclass(+)

D(x)<0 [symbol] xεclass(−)

D(x)=0, decision boundary.

Decision functions that are simple weighted sums of the training patterns plus a bias are called linear discriminant functions.

D(x)=w·x+b,

where w is the weight vector and b is a bias value.

In the case of Golub's classifier, each weight is computed as:

Wᵢ=(μᵢ(+)−μᵢ(−))/(σᵢ(+)+σᵢ(−)),

where (μᵢ and σᵢ are the mean and standard deviation of the gene expression values of gene i for all the patients of class (+) or class (−), i=1, . . . n. Large positive wᵢ values indicate strong correlation with class (+) whereas large negative wᵢ values indicate strong correlation with class (−). Thus the weights can also be used to rank the features (genes) according to relevance. The bias is computed as b=−w·μ, where μ=(μ(+)+μ(−))/2.

Golub's classifier is a standard reference that is robust against outliers. Once a first classifier is trained, the magnitude of wᵢ is used to rank the genes. The classifiers are then retrained with subsets of genes of different sizes, including the best ranking genes.

To assess the statistical significance of the results, ten random splits of the data including samples were prepared from either preparation method and submitted to the same method. This allowed the computation of an average and standard deviation for comparison purposes.

Tissue from the same patient was processed either directly (unfiltered) or after the LCM procedure, yielding a pair of microarray experiments. This yielded 13 pairs, including: four G4; one G3+4; two G3; four BPH; one CZ (normal) and one PZ (normal).

For each data preparation method (LCM or unfiltered tissues), the tissues were grouped into two subsets:
Cancer=G4+G3 (7 cases)
Normal=BPH+CZ+PZ (6 cases).

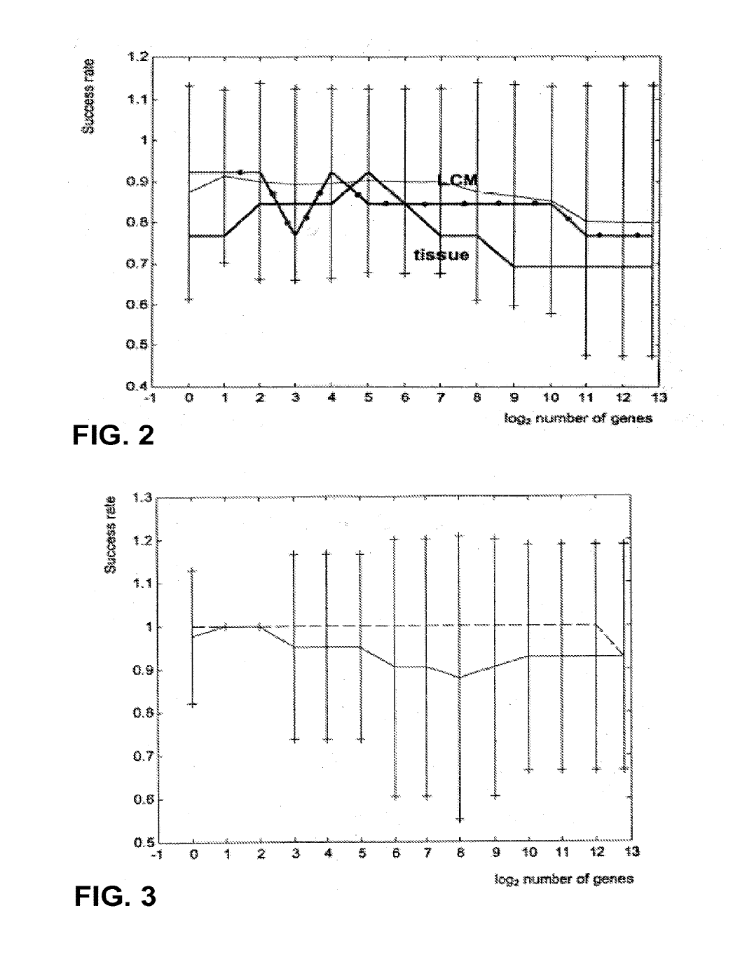

The results are shown in FIG. 2. The large error bars are due to the small size. However, there is an indication that LCM samples are better than unfiltered tissue samples. It is also interesting to note that the average curve corresponding to random splits of the data is above both curves. This is not surprising since the data in subset 1 and subset 2 are differently distributed. When making a random split rather than segregating samples, both LCM and unfiltered tissues are represented in the training and the test set and performance on the test set are better on average.

The same methods were applied to determine whether microarrays with gene expression data rejected by the Affymetrix quality criterion contained useful information by focusing on the problem of separating BPH tissue vs. G4 tissue with a total of 42 arrays (18 BPH and 24 G4).

The Affymetrix criterion identified 17 good quality arrays, 8 BPH and 9 G4. Two subsets were formed:
Subset 1=“good” samples, 8 BPH+9 G4
Subset 2=“mediocre” samples, 10 BPH+15 G4

For comparison, all of the samples were lumped together and 10 random subset 1 containing 8 BPH+9 G4 of any quality were selected. The remaining samples were used as subset 2 allowing an average curve to be obtained. Additionally the subsets were inverted with training on the “mediocre” examples and testing on the “good” examples.

When the mediocre samples are trained, perfect accuracy on the good samples is obtained, whereas training on the good examples and testing on the mediocre yield substantially worse results.

All the BPH and G4 samples were divided into LCM and unfiltered tissue subsets to repeat similar experiments as in the previous Section:
Subset1=LCM samples (5 BPH+6 LCM)
Subset2=unfiltered tissue samples (13 BPH+18 LCM)

There, in spite of the difference in sample size, training on LCM data yields better results. In spite of the large error bars, this is an indication that the LCM data preparation method might be of help in improving sample quality.

BPH vs. G4

The Affymetrix data quality criterion were irrelevant for the purpose of determining the predictive value of particular genes and while the LCM samples seemed marginally better than the unfiltered samples, it was not possible to determine a statistical significance. Therefore, all samples were grouped together and the separation BPH vs. G4 with all 42 samples (18 BPH and 24 G4) was preformed.

To evaluate performance and compare Golub's method with SVMs, the leave-one-out method was used. The fraction of successfully classified left-out examples gives an estimate of the success rate of the various classifiers.

In this procedure, the gene selection process was run 41 times to obtain subsets of genes of various sizes for all 41 gene rankings. One classifier was then trained on the corresponding 40 genes for every subset of genes. This leave-one-out method differs from the “naive” leave-one-out that consists of running the gene selection only once on all 41 examples and then training 41 classifiers on every subset of genes. The naive method gives overly optimistic results because all the examples are used in the gene selection process, which is like “training on the test set”. The increased accuracy of the first method is illustrated in FIG. 3. The method used in the figure is SVM-RFE and the classifier used is an SVM. All SVMs are linear with soft margin parameters C=100 and t=10¹⁴. The dashed line represents the “naive” leave-one-out (loo), which consists in running the gene selection once and performing loo for classifiers using subsets of genes thus derived, with different sizes. The solid line represents the more computationally expensive “true” loo, which consists in running the gene selection 41 times, for every left out example. The left out example is classified with a classifier trained on the corresponding 40 examples for every selection of genes. If f is the success rate obtained (a point on the curve), the standard deviation is computed as sqrt(f(1−f)).

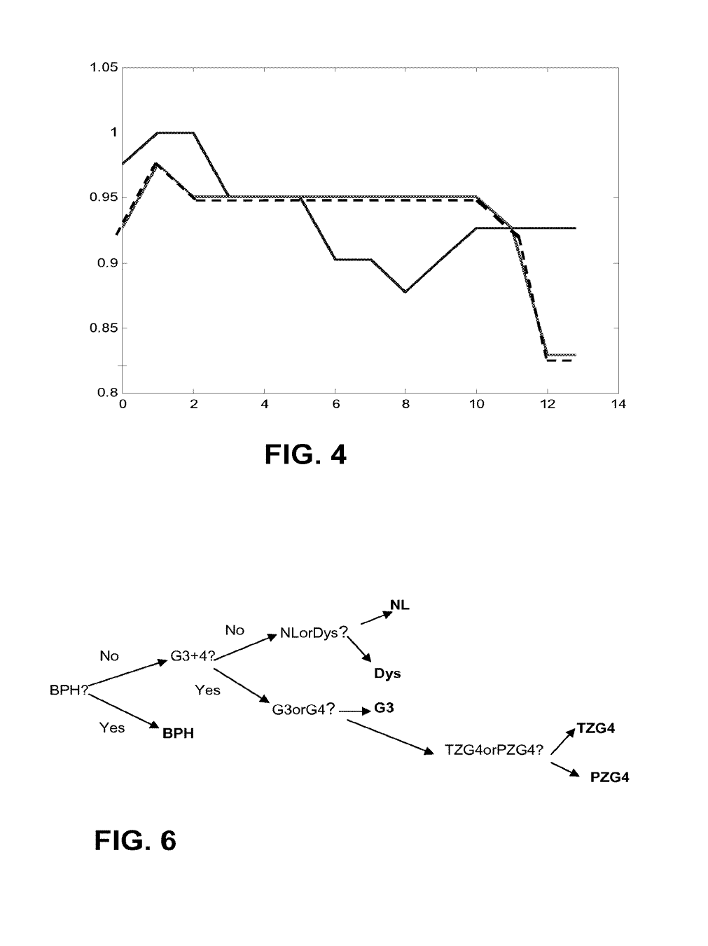

The “true” leave-one-out method was used to evaluate both Golub's method and SVMs. The results are shown in FIG. 4. SVMs outperform Golub's method for the small number of examples. However, the difference is not statistically significant in a sample of this size (1 error in 41 examples, only 85% confidence that SVMs are better).

### Example 2

### Analyzing Small Data Sets with Multiple Features

Small data sets with large numbers of features present several problems. In order to address ways of avoiding data overfitting and to assess the significance in performance of multivariate and univariate methods, the samples from Example 1 that were classified by Affymetrix as high quality samples were further analyzed. The samples included 8 BPH and 9 G4 tissues. Each microarray recorded 7129 gene expression values. About ⅔ of the samples in the BPH/G4 subset were considered of inadequate quality for use with standard non-SVM methods.

Simulations resulting from multiple splits of the data set of 17 examples (8 BPH and 9 G4) into a training set and a test set were run. The size of the training set is varied. For each training set drawn, the remaining data are used for testing. For number of training examples greater than 4 and less than 16, 20 training sets were selected at random. For 16 training examples, the leave-one-out method was used, in that all the possible training sets obtained by removing 1 example at a time (17 possible choices) were created. The test set is then of size 1. Note that the test set is never used as part of the feature selection process, even in the case of the leave-one-out method.

For 4 examples, all possible training sets containing 2 examples of each class (2 BPH and 2 G4), were created and 20 of them were selected at random. For SVM methods, the initial training set size is 2 examples, one of each class (1 BPH and 1 G4). The examples of each class are drawn at random. The performance of the LDA methods cannot be computed with only 2 examples, because at least 4 examples (2 of each class) are required to compute intraclass standard deviations. The number of training examples is incremented by steps of 2.

The top ranked genes are presented in Tables 5-8. Having determined that the SVM method provided the most compact set of features to achieve 0 leave-one-out error and that the SF-SVM method is the best and most robust method for small numbers of training examples, the top genes found by these methods were researched in the literature. Most of the genes have a connection to cancer or more specifically to prostate cancer.

Table 5 shows the top ranked genes for SF LDA using 17 best BPH/G4.

TABLE 5

| Rank | GAN | EXP | Description |
|---|---|---|---|
| 10 | X83416 | −1 | H. sapiens PrP gene |
| 9 | U50360 | −1 | Human calcium calmodulin-dependent protein |
| | | | kinase II gamma mRNA |
| 8 | U35735 | −1 | Human RACH1 (RACH1) mRNA |
| 7 | M57399 | −1 | Human nerve growth factor (HBNF-1) mRNA |
| 6 | M55531 | −1 | Human glucose transport-like 5 (GLUT5) |
| | | | mRNA |
| 5 | U48959 | −1 | Human myosin light chain kinase (MLCK) |
| | | | mRNA |
| 4 | Y00097 | −1 | Human mRNA for protein p68 |
| 3 | D10667 | −1 | Human mRNA for smooth muscle myosin |
| | | | heavy chain |
| 2 | L09604 | −1 | Homo sapiens differentiation-dependent |
| | | | A4 protein MRNA |
| 1 | HG1612- | 1 | McMarcks |
| | HT1612 | | |

where GAN = Gene Acession Number; EXP = Expression (−1 = underexpressed in cancer (G4) tissues; +1 = overexpressed in cancer tissues).

Table 6 lists the top ranked genes obtained for LDA using 17 best BPH/G4.

TABLE 6

| Rank | GAN | EXP | Description |
|---|---|---|---|
| 10 | J03592 | 1 | Human ADP/ATP translocase mRNA |
| 9 | U40380 | 1 | Human presenilin I-374 (AD3-212) mRNA |
| 8 | D31716 | −1 | Human mRNA for GC box bindig protein |
| 7 | L24203 | −1 | Homo sapiens ataxia-telangiectasia group D |
| 6 | J00124 | −1 | Homo sapiens 50 kDa type I epidermal keratin |
| | | | gene |
| 5 | D10667 | −1 | Human mRNA for smooth muscle myosin heavy |
| | | | chain |
| 4 | J03241 | −1 | Human transforming growth factor-beta 3 |
| | | | (TGF-beta3) MRNA |
| 3 | 017760 | −1 | Human laminin S B3 chain (LAMB3) gene |
| 2 | X76717 | −1 | H. sapiens MT-11 mRNA |
| 1 | X83416 | −11 | H. sapiens PrP gene |

Table 7 lists the top ranked genes obtained for SF SVM using 17 best BPH/G4.

TABLE 7

| Rank | GAN | EXP | Description |
|---|---|---|---|
| 10 | X07732 | 1 | Human hepatoma mRNA for serine protease |
| | | | hepsin |
| 9 | J03241 | −1 | Human transforming growth factor-beta 3 (TGF- |
| | | | beta3) MRNA |
| 8 | X83416 | −1 | H. sapiens PrP gene |
| 7 | X14885 | −1 | H. sapiens gene for transforming growth factor- |
| | | | beta 3 (TGF-beta 3) exon 1 (and joined CDS) |
| 6 | U32114 | −1 | Human caveolin-2 mRNA |
| 5 | M16938 | 1 | Human homeo-box c8 protein |
| 4 | L09604 | −1 | H. sapiens differentiation-dependent A4 protein |
| | | | MRNA |
| 3 | Y00097 | −1 | Human mRNA for protein p68 |
| 2 | D88422 | −1 | Human DNA for cystatin A |
| 1 | U35735 | −1 | Human RACH1 (RACH1) mRNA |

Table 8 provides the top ranked genes for SVM using 17 best BPH/G4.

TABLE 8

| Rank | GAN | EXP | Description |
|---|---|---|---|
| 10 | X76717 | −1 | H. sapiens MT-11 mRNA |
| 9 | U32114 | −1 | Human caveolin-2 mRNA |
| 8 | X85137 | 1 | H. sapiens mRNA for kinesin-related protein |
| 7 | D83018 | −1 | Human mRNA for nel-related protein 2 |
| 6 | D10667 | −1 | Human mRNA for smooth muscle myosin |
| | | | heavy chain |
| 5 | M16938 | 1 | Human homeo box c8 protein |
| 4 | L09604 | −1 | Homo sapiens differentiation-dependent A4 |
| | | | protein mRNA |
| 3 | HG1612 | 1 | McMarcks |
| 2 | M10943 | −1 | Human metaIlothionein-If gene (hMT-If) |
| 1 | X83416 | −1 | H. sapiens PrP gene |

Using the “true” leave-one-out method (including gene selection and classification), the experiments indicate that 2 genes should suffice to achieve 100% prediction accuracy. The two top genes were therefore more particularly researched in the literature. The results are summarized in Table 10. It is interesting to note that the two genes selected appear frequently in the top 10 lists of Tables 5-8 obtained by training only on the 17 best genes.

Table 9 is a listing of the ten top ranked genes for SVM using all 42 BPH/G4.

TABLE 9

| Rank | GAN | EXP | Description |
|---|---|---|---|
| 10 | X87613 | −1 | H. sapiens mRNA for skeletal muscle abundant |
| 9 | X58072 | −1 | Human hGATA3 mRNA for trans-acting T-cell |
| | | | specific |
| 8 | M33653 | −1 | Human alpha-2 type IV collagen (COL4A2) |
| 7 | S76473 | 1 | trkB [human brain mRNA] |
| 6 | X14885 | −1 | H. sapiens gene for transforming growth factor- |
| | | | beta 3 (TGF-beta 3) exon 1 (and joined CDS) |
| 5 | S83366 | −1 | region centromeric to t(12; 17) brakepoint |
| 4 | X15306 | −1 | H. sapiens NF-H gene |
| 3 | M30894 | 1 | Human T-cell receptor Ti rearranged gamma- |
| | | | chain |
| 2 | M16938 | 1 | Human homeo box c8 protein |
| 1 | U35735 | −1 | Human RACH1 (RACH1) mRNA |

Table 10 provides the findings for the top 2 genes found by SVM using all 42 BPH/G4. Taken together, the expression of these two genes is indicative of the severity of the disease.

TABLE 10

| GAN | Synonyms | Possible function/link to prostate cancer |
|---|---|---|
| M16938 | HOXC8 | Hox genes encode transcriptional regulatory |
| | | proteins that are largely responsible for |
| | | establishing the body plan of all metazoan |
| | | organisms. There are hundreds of papers in |
| | | PubMed reporting the role of HOX genes in |
| | | various cancers. HOXC5 and HOXC8 |
| | | expression are selectively turned on in human |
| | | cervical cancer cells compared to normal |
| | | keratinocytes. Another homeobox gene (GBX2) |
| | | may participate in metastatic progression in |
| | | prostatic cancer. Another HOX protein (hoxb- |
| | | 13) was identified as an androgen-independent |
| | | gene expressed in adult mouse prostate epithelial |
| | | cells. The authors indicate that this provides a |
| | | new potential target for developing therapeutics |
| | | to treat advanced prostate cancer |
| U35735 | Jk | Overexpression of RACH2 in human tissue |
| | Kidd | culture cells induces apoptosis. RACH1 is |
| | RACH1 | downregulated in breast cancer cell line MCF-7. |
| | RACH2 | RACH2 complements the RAD1 protein. RAM |
| | SLC14A1 | is implicated in several cancers. Significant |
| | UT1 | positive lod scores of 3.19 for linkage of the Jk |
| | UTE | (Kidd blood group) with cancer family |
| | | syndrome (CFS) were obtained. CFS gene(s) |
| | | may possibly be located on chromosome 2, |
| | | where Jk is located. |

Table 11 shows the severity of the disease as indicated by the top 2 ranking genes selected by SVMs using all 42 BPH and G4 tissues.

TABLE 11

| | HOXC8 | HOXC8 |
|---|---|---|
| | Underexpressed | Overexpressed |
| RACH1 Overexpressed | Benign | N/A |
| RACH1 Underexpressed | Grade 3 | Grade 4 |

### Example 3

### Prostate Cancer Study on Affymetrix Gene Expression Data (09-2004)

A set of Affymetrix microarray GeneChip experiments from prostate tissues were obtained from Professor Stamey at Stanford University. The data statistics from samples obtained for the prostate cancer study are summarized in Table 12 (which lists the same data as in Table 3 but organized differently.) Preliminary investigation of the data included determining the potential need for normalizations. Classification experiments were run with a linear SVM on the separation of Grade 4 tissues vs. BPH tissues. In a 32×3-fold experiment, an 8% error rate could be achieved with a selection of 100 genes using the multiplicative updates technique (similar to RFE-SVM). Performances without feature selection are slightly worse but comparable. The gene most often selected by forward selection was independently chosen in the top list of an independent published study, which provided an encouraging validation of the quality of the data.

TABLE 12

| Prostate zone | Histological classification | No. of samples |
|---|---|---|
| Central (CZ) | Normal (NL) | 9 |
| | Dysplasia (Dys) | 4 |
| | Grade 4 cancer (G4) | 1 |
| Peripheral (PZ) | Normal (NL) | 13 |
| | Dysplasia (Dys) | 13 |
| | Grade 3 cancer (G3) | 11 |
| | Grade 4 cancer (G4) | 18 |
| Transition (TZ) | Benign Prostate Hyperplasia (BPH) | 10 |
| | Grade 4 cancer (G4) | 8 |
| Total | | 87 |

As controls, normal tissues and two types of abnormal tissues are used in the study: BPH and Dysplasia.

To verify the data integrity, the genes were sorted according to intensity. For each gene, the minimum intensity across all experiments was taken. The top 50 most intense values were taken. Heat maps of the data matrix were made by sorting the lines (experiments) according to zone, grade, and time processed. No correlation was found with zone or grade, however, there was a significant correlation with the time the sample was processed. Hence, the arrays are poorly normalized.

In other ranges of intensity, this artifact is not seen. Various normalization techniques were tried, but no significant improvements were obtained. It has been observed by several authors that microarray data are log-normal distributed. A qqplot of all the log of the values in the data matrix confirms that the data are approximately log-normal distributed. Nevertheless, in preliminary classification experiments, there was not a significant advantage of taking the log.

Tests were run to classify BPH vs. G4 samples. There were 10 BPH samples and 27 G4 samples. 32×3 fold experiments were performed in which the data was split into 3 subsets 32 times. Two of the subsets were used for training while the third was used for testing. The results were averaged. A feature selection was performed for each of the 32×3 data splits; the features were not selected on the entire dataset.

A linear SVM was used for classification, with ridge parameter 0.1, adjusted for each class to balance the number of samples per class. Three feature selection methods were used: (1) multiplicative updates down to 100 genes (MU100); (2) forward selection with approximate gene orthogonalisation up to 2 genes (FS2); and (3) no gene selection (NO).

The data was either raw or after taking the log (LOG). The genes were always standardized (STD: the mean over all samples is subtracted and the result is divided by the standard deviation; mean and stdev are computed on training data only, the same coefficients are applied to test data).

The results for the performances for the BPH vs. G4 separation are shown in Table 13 below, with the standard errors are shown in parentheses. “Error rate” is the average number of misclassification errors; “Balanced errate” is the average of the error rate of the positive class and the error rate of the negative class; “AUC” is the area under the ROC (receiver operating characteristic) curves that plots the sensitivity (error rate of the positive class, G4) as a function of the specificity (error rate of the negative class, BPH).

It was noted that the SVM performs quite well without feature selection, and MU 100 performs similarly, but slightly better. The number of features was not adjusted—100 was chosen arbitrarily.

TABLE 13

| | | | Balanced | |
|---|---|---|---|---|
| Preprocessing | Feat. Select. | Error rate | errate | AUC |
| Log + STD | MU 100 | 8.09 (0.66) | 11.68 (1.09) | 98.93 (0.2) |
| Log + STD | FS 2 | 13.1 (1.1) | 15.9 (1.3) | 92.02 (1.15) |
| Log + STD | No selection | 8.49 (0.71) | 12.37 (1.13) | 97.92 (0.33) |
| STD | No selection | 8.57 (0.72) | 12.36 (1.14) | 97.74 (0.35) |

In Table 13, the good AUC and the difference between the error rate and the balanced error rate show that the bias of the classifier must be optimized to obtained a desired tradeoff between sensitivity and specificity.

Two features are not enough to match the best performances, but do quite well already.

It was determined which features were selected most often with the FS 2 method. The first gene (3480) was selected 56 times, while the second best one (5783) was selected only 7 times. The first one is believed to be relevant to cancer, while the second one has probably been selected for normalization purposes. It is interesting that the first gene (Hs.79389) is among the top three genes selected in another independent study (Febbo-Sellers, 2003).

The details of the two genes are as follows:
Gene 3480: gb:NM_{—}006159.1/DEF=Homo sapiens nel (chicken)-like 2 (NELL2), mRNA. /FEA=mRNA /GEN=NELL2/PROD=nel (chicken)-like2 /DB_XREF=gi:5453765/UG=Hs.79389 nel (chicken)-like 2 /FL=gb:D83018.1 gb:NM_{—}006159.1
Gene 5783: gb:NM_{—}018843.1/DEF=Homo sapiens mitochondrial carrier family protein(L0055972), mRNA. /FEA=mRNA /GEN=L0055972 /PROD=mitochondrial carrier family protein /DB_XREF=gi:10047121 /UG=Hs.172294 mitochondrial carrier family protein /FL=gb:NM_{—}018843.1 gb:AF125531.1.

### Example 4

### Prostate Cancer Study from Affymetrix Gene Expression Data (10-2004)

This example is a continuation of the analysis of Example 3 above on the Stamey prostate cancer microarray data. PSA has long been used as a biomarker of prostate cancer in serum, but is no longer useful. Other markers have been studied in immunohistochemical staining of tissues, including p27, Bcl-2, E-catherin and P53. However, to date, no marker has gained use in routine clinical practice.

The gene rankings obtained correlate with those of the Febbo paper, confirming that the top ranking genes found from the Stamey data have a significant intersection with the genes found in the Febbo study. In the top 1000 genes, about 10% are Febbo genes. In comparison, a random ordering would be expected to have less than 1% are Febbo genes.

BPH is not by itself an adequate control. When selecting genes according to how well they separate grade 4 cancer tissues (G4) from BPH, one can find genes that group all non-BPH tissues with the G4 tissues (including normal, dysplasia and grade 3 tissues). However, when BPH is excluded from the training set, genes can be found that correlate well with disease severity. According to those genes, BPH groups with the low severity diseases, leading to a conclusion that BPH has its own molecular characteristics and that normal adjacent tissues should be used as controls.

TZG4 is less malignant than PZG4. It is known that TZ cancer has a better prognosis than PZ cancer. The present analysis provides molecular confirmation that TZG4 is less malignant than PZG4. Further, TZG4 samples group with the less malignant samples (grade 3, dysplasia, normal, or BPH) than with PZG4. This differentiated grouping is emphasized in genes correlating with disease progression (normal<dysplasia<g3<g4) and selected to provide good separation of TZG4 from PZG4 (without using an ordering for TZG4 and PZG4 in the gene selection criterion).

Ranking criteria implementing prior knowledge about disease malignancy are more reliable. Ranking criteria validity was assessed both with p values and with classification performance. The criterion that works best implements a tissue ordering normal<dysplasia<G3<G4 and seeks a good separation TZG4 from PZG4. The second best criterion implements the ordering normal<dysplasia<G3<TZG4<PZG4.

Comparing with other studies may help reducing the risk of overfitting. A subset of 7 genes was selected that ranked high in the present study and that of Febbo et al. 2004. Such genes yield good separating power for G4 vs. other tissues. The training set excludes BPH samples and is used both to select genes and train a ridge regression classifier. The test set includes 10 BPH and 10 G4 samples (½ from the TZ and ½ from the PZ). Success was evaluated with the area under the ROC curve (“AUC”)(sensitivity vs. specificity) on test examples. AUCs between 0.96 and 1 are obtained, depending on the number of genes. Two genes are of special interest (GSTP1 and PTGDS) because they are found in semen and could be potential biomarkers that do not require the use of biopsied tissue.

The choice of the control may influence the findings (normal tissue or BPH). as may the zones from which the tissues originate. The first test sought to separate Grade 4 from BPH. Two interesting genes were identified by forward selection as gene 3480 (NELL2) and gene 5783(L0055972). As explained in Example 3, gene 3480 is the informative gene, and it is believed that gene 5783 helps correct local on-chip variations. Gene 3480, which has Unigene cluster id. Hs.79389, is a Nel-related protein, which has been found at high levels in normal tissue by Febbo et al.

All G4 tissues seem intermixed regardless of zone. The other tissues are not used for gene selection and they all fall on the side of G4. Therefore, the genes found characterize BPH, not G4 cancer, such that it is not sufficient to use tissues of G4 and BPH to find useful genes to characterize G4 cancer.

For comparison, two filter methods were used: the Fisher criterion and the shrunken centroid criterion (Tibshirani et al, 2002). Both methods found gene 3480 to be highly informative (first or second ranking). The second best gene is 5309, which has Unigene cluster ID Hs. 100431 and is described as small inducible cytokine B subfamily (Cys-X-Cys motif). This gene is highly correlated to the first one.

The Fisher criterion is implemented by the following routine:
A vector x containing the values of a given feature for all patt_num samples
cl_num classes, k=1, 2, . . . cl_num, grouping the values of x
mu_val(k) is the mean of the x values for class k
var_val(k) is the variance of the x values for class k
patt_per_class(k) is the number of elements of class k
Unbiased_within_var is the unbiased pooled within class variance, i.e., we make a weighted average of var_val(k) with coefficients
patt_per_class(k)/(patt_num−cl_num)
Unbiased_between_var=var(mu_val); % Divides by cl_num−1 then
Fisher_crit=Unbiased_between_var /Unbiased_within_var

Although the shrunken centroid criterion is somewhat more complicated than the Fisher criterion, it is quite similar. In both cases, the pooled within class variance is used to normalize the criterion. The main difference is that instead of ranking according to the between class variance (that is, the average deviation of the class centroids to the overall centroid), the shrunken centroid criterion uses the maximum deviation of any class centroid to the global centroid. In doing so, the criterion seeks features that well separate at least one class, instead of features that well separate all classes (on average).

The other small other differences are:
A fudge factor is added to Unbiased_within_std=sqrt(Unbiased_within_var) to prevent divisions by very small values. The fudge factor is computed as: fudge=mean(Unbiased_within_std); the mean being taken over all the features.
Each class is weighted according to its number of elements cl_elem(k). The deviation for each class is weighted by 1/sqrt(1/cl_elem(k)+1/patt_num). Similar corrections could be applied to the Fisher criterion.

The two criteria are compared using pvalues. The Fisher criterion produces fewer false positive in the top ranked features. It is more robust, however, it also produces more redundant features. It does not find discriminant features for the classes that are least abundant or hardest to separate.

Also for comparison, the criterion of Golub et al., also known as signal to noise ratio, was used. This criterion is used in the Febbo paper to separate tumor vs. normal tissues. On this data that the Golub criterion was verified to yield a similar ranking as the Pearson correlation coefficient. For simplicity, only the Golub criterion results are reported. To mimic the situation, three binary separations were run: (G3+4 vs. all other tissues), (G4 vs. all other tissues), and (G4 vs. BPH). As expected, the first gene selected for the G4 vs. BPH is 3480, but it does not rank high in the G3+4 vs. all other and G4 vs. all other.

Compared to a random ranking, the genes selected using the various criteria applied are enriched in Febbo genes, which cross-validates the two study. For the multiclass criteria, the shrunken centroid method provides genes that are more different from the Febbo genes than the Fisher criterion. For the two-class separations, the tumor vs normal (G3+4 vs others) and the G4 vs. BPH provide similar Febbo enrichment while the G4 vs. all others gives gene sets that depart more from the Febbo genes. Finally, it is worth noting that the initial enrichment up to 1000 genes is of about 10% of Febbo genes in the gene set. After that, the enrichment decreases. This may be due to the fact that the genes are identified by their Unigene Ids and more than one probe is attributed to the same Id. In any case, the enrichment is very significant compared to the random ranking.

A number of probes do not have Unigene numbers. Of 22,283 lines in the Affymetrix data, 615 do not have Unigene numbers and there are only 14,640 unique Unigene numbers. In 10,130 cases, a unique matrix entry corresponds to a particular Unigene ID. However, 2,868 Unigene IDs are represented by 2 lines, 1,080 by 3 lines, and 563 by more than 3 lines. One Unigene ID covers 13 lines of data. For example, Unigene ID Hs.20019, identifies variants of Homo sapiens hemochromatosis (HFE) corresponding to GenBank assession numbers: AF115265.1, NM_{—}000410.1, AF144240.1, AF150664.1, AF149804.1, AF144244.1, AF115264.1, AF144242.1, AF144243.1, AF144241.1, AF079408.1, AF079409.1, and (consensus) BG402460.

The Unigene IDs of the paper of Febbo et al. (2003) were compared using the U95AV2 Affymetrix array and the IDs found in the U133A array under study. The Febbo paper reported 47 unique Unigene IDs for tumor high genes, 45 of which are IDs also found in the U133A array. Of the 49 unique Unigene IDs for normal high genes, 42 are also found in the U133A array. Overall, it is possible to see cross-correlations between the findings. There is a total of 96 Febbo genes that correspond to 173 lines (some genes being repeated) in the current matrix.

Based on the current results, one can either conclude that the “normal” tissues that are not BPH and drawn near the cancer tissues are on their way to cancer, or that BPH has a unique molecular signature that, although it may be considered “normal”, makes it unfit as a control. A test set was created using 10 BPH samples and 10 grade 4 samples. Naturally, all BPH are in the TZ. The grade 4 are ½ in the TZ and ½ in the PZ.

Gene selection experiments were performed using the following filter methods:

(1)—Pearsons correlation coefficient to correlate with disease severity, where disease severity is coded as normal=1, dysplasia=2, grade3=3, grade4=4.

(2)—Fisher's criterion to separate the 4 classes (normal, dysplasia, grade3, grade4) with no consideration of disease severity.

(3)—Fisher's criterion to separate the 3 classes (PZ, CZ, TZ)

(4)—Relative Fisher criterion by computing the ratio of the between class variances of the disease severity and the zones, in an attempt to de-emphasize the zone factor.

(5)—Fisher's criterion to separate 8 classes corresponding to all the combinations of zones and disease severity found in the training data.

(6)—Using the combination of 2 rankings: the ranking of (1) and a ranking by zone for the grade 4 samples only. The idea is to identify genes that separate TZ from PZ cancers that have a different prognosis.

For each experiment, scatter plots were analyzed for the two best selected genes, the heat map of the 50 top ranked genes was reviewed, and p values were compared. The conclusions are as follows:

The Pearson correlation coefficient tracking disease severity (Experiment (1)) gives a similar ranking to the Fisher criterion, which discriminates between disease classes without ranking according to severity. However, the Pearson criterion has slightly better p values and, therefore, may give fewer false positives. The two best genes found by the Pearson criterion are gene 6519, ranked 6^{th} by the Fisher criterion, and gene 9457, ranked 1^{st} by the Fisher criterion. The test set examples are nicely separated, except for one outlier.

The zonal separation experiments were not conclusive because there are only 3 TZ examples in the training set and no example of CZ in the test set. Experiment (3) revealed a good separation of PZ and CZ on training data. TZ was not very well separated. Experiments (4) and (5) did not show very significant groupings. Experiment (6) found two genes that show both disease progression and that TZ G4 is grouped with “less severe diseases” than PZ G4, although that constraint was not enforced. To confirm the latter finding, the distance for the centroids of PZG4 and TZG4 were compared to control samples. Using the test set only (controls are BPH), 63% of all the genes show that TZG4 is closer to the control than PZG4. That number increases to 70% if the top 100 genes of experiment (6) are considered. To further confirm, experiment (6) was repeated with the entire dataset (without splitting between training and test). TZG4 is closer to normal than PZG4 for most top ranked genes. In the first 15 selected genes, 100% have TZG4 closer to normal than PZG4. This finding is significant because TZG4 has better prognosis than PZG4.

Classification experiments were performed to assess whether the appropriate features had been selected using the following setting:

The data were split into a training set and a test set. The test set consists of 20 samples: 10 BPH, 5 TZG4 and 5 PZG4. The training set contains the rest of the samples from the data set, a total of 67 samples (9 CZNL, 4 CZDYS, 1 CZG4, 13 PZNL, 13 PZDYS, 11 PZG3, 13 PZG4, 3 TZG4). The training set does not contain any BPH.

Feature selection was performed on training data only. Classification was performed using linear ridge regression. The ridge value was adjusted with the leave-one-out error estimated using training data only. The performance criterion was the area under the ROC curve (AUC), where the ROC curve is a plot of the sensitivity as a function of the specificity. The AUC measures how well methods monitor the tradeoff sensitivity/specificity without imposing a particular threshold.

P values are obtained using a randomization method proposed by Tibshirani et al. Random “probes” that have a distribution similar to real features (gene) are obtained by randomizing the columns of the data matrix, with samples in lines and genes in columns. The probes are ranked in a similar manner as the real features using the same ranking criterion. For each feature having a given score s, where a larger score is better, a p value is obtained by counting the fraction of probes having a score larger than s. The larger the number of probes, the more accurate the p value.

For most ranking methods, and for forward selection criteria using probes to compute p values does not affect the ranking. For example, one can rank the probes and the features separately for the Fisher and Pearson criteria.

P values measure the probability that a randomly generated probe imitating a real gene, but carrying no information, gets a score larger or equal to s. Considering a single gene, if it has a score of s, the p value test can be used to test whether to reject the hypothesis that it is a random meaningless gene by setting a threshold on the p value, e.g., 0.0. The problem is that there are many genes of interest (in the present study, N=22,283.) Therefore, it becomes probable that at least one of the genes having a score larger than s will be meaningless. Considering many genes simultaneously is like doing multiple testing in statistics. If all tests are independent, a simple correction known as the Bonferroni correction can be performed by multiplying the p values by N. This correction is conservative when the test are not independent.

From p values, one can compute a “false discovery rate” as FDR(s)=pvalue(s)*N/r, where r is the rank of the gene with score s, pvalue(s) is the associated p value, N is the total number of genes, and pvalue(s)*N is the estimated number of meaningless genes having a score larger than s. FDR estimates the ratio of the number of falsely significant genes over the number of genes call significant.

Of the classification experiments described above, the method that performed best was the one that used the combined criteria of the different classification experiments. In general, imposing meaningful constraints derived from prior knowledge seems to improve the criteria. In particular, simply applying the Fisher criterion to the G4 vs. all-the-rest separation (G4vsAll) yields good separation of the training examples, but poorer generalization than the more constrained criteria. Using a number of random probes equal to the number of genes, the G4vsAll identifies 170 genes before the first random probe, multiclass Fisher obtains 105 and the Pearson criterion measuring disease progression gets 377. The combined criteria identifies only 8 genes, which may be attributed to the different way in which values are computed. With respect to the number of Febbo genes found in the top ranking genes, G4 vs All has 20, multiclass Fisher 19, Pearson 19, and the combined criteria 8. The combined criteria provide a characterization of zone differentiation. On the other hand, the top 100 ranking genes found both by Febbo and by criteria G4 vs All, Fisher or Pearson have a high chance of having some relevance to prostate cancer. These genes are listed in Table 14.

TABLE 14

| | Unigene | | | G4 vs | | |
|---|---|---|---|---|---|---|
| Order Num | ID | Fisher | Pearson | ALL | AUC | Description |
| 12337 | Hs.7780 | 11 | 6 | 54 | 0.96 | cDNA DKFZp56A072 |
| 893 | Hs.226795 | 17 | 7 | 74 | 0.99 | Glutathione S-transferase pi (GSTP1) |
| 5001 | Hs.823 | 41 | 52 | 72 | 0.96 | Hepsin (transmembrance protease, |
| | | | | | | serine 1) (HPN) |
| 1908 | Hs.692 | 62 | 34 | 111 | 0.96 | Tumor-associated calcium signal |
| | | | | | | transducer 1 (TACSTD1) |
| 5676 | Hs.2463 | 85 | 317 | 151 | 1 | Angiopoietin 1 (ANGPT1) |
| 12113 | Hs.8272 | 181 | 93 | 391 | 1 | Prostaglandin D2 synthase (21 kD, |
| | | | | | | brain) (PTGDS) |
| 12572 | Hs.9651 | 96 | 131 | 1346 | 0.99 | RAS related viral oncogene homolog |
| | | | | | | (RRAS) |

Table 14 shows genes found in the top 100 as determined by the three criteria, Fisher, Pearson and G4vsALL, that were also reported in the Febbo paper. In the table, Order num is the order in the data matrix. The numbers in the criteria columns indicate the rank. The genes are ranked according to the sum of the ranks of the 3 criteria. Classifiers were trained with increasing subset sizes showing that a test AUC of 1 is reached with 5 genes.

The published literature was checked for the genes listed in Table 14. Third ranked Hepsin has been reported in several papers on prostate cancer: Chen et al. (2003) and Febbo et al. (2003) and is picked up by all criteria. Polymorphisms of second ranked GSTP1 (also picked by all criteria) are connected to prostate cancer risk (Beer et al, 2002). The fact that GSTP1 is found in semen (Lee (1978)) makes it a potentially interesting marker for non-invasive screening and monitoring. The clone DKFZp564A072, ranked first, is cited is several gene expression studies.

Fourth ranked Gene TACSTD1 was also previously described as more-highly expressed in prostate adenocarcinoma (see Lapointe et al, 2004 and references therein). Angiopoietin (ranked fifth) is involved in angiogenesis and known to help the blood irrigation of tumors in cancers and, in particular, prostate cancer (see e.g. Cane, 2003). Prostaglandin D2 synthase (ranked sixth) has been reported to be linked to prostate cancer in some gene expression analysis papers, but more interestingly, prostaglandin D synthase is found in semen (Tokugawa, 1998), making it another biomarker candidate for non-invasive screening and monitoring. Seventh ranked RRAS is an oncogene, so it makes sense to find it in cancer, however, its role in prostate cancer has not been documented.

A combined criterion was constructed for selecting genes according to disease severity NL<DYS<G3<G4 and simultaneously tries to differentiate TZG4 from PZG4 without ordering them. This following procedure was used:
Build an ordering using the Pearson criterion with encoded target vector having values NL=1, DYS=2, G3=3, G4=4 (best genes come last.)
Build an ordering using the Fisher criterion to separate TZG4 from PZG$ (best genes come last.)
Obtain a combined criterion by adding for each gene its ranks obtained with the first and second criterion.
Sort according to the combined criterion (in descending order, best first).
   P values can be obtained for the combined criterion as follows:
Unsorted score vectors for real features (genes) and probes are concatenated for both criteria (Pearson and Fisher).
Genes and probes are sorted together for both criteria, in ascending order (best last).
The combined criterion is obtained by summing the ranks, as described above.
For each feature having a given combined criterion value s (larger values being better), a p value is obtained by counting the fraction of probes a having a combined criterion larger than s.

Note that this method for obtaining p values disturbs the ranking, so the ranking that was obtained without the probes listed in Table 15 was used.

A listing of genes obtained with the combined criterion are shown in Table 15. The ranking is performed on training data only. “Order num” designates the gene order number in the data matrix; p values are adjusted by the Bonferroni correction; “FDR” indicates the false discovery rate; “Test AUC” is the area under the ROC curve computed on the test set; and “Cancer cor” indicates over-expression in cancer tissues.

TABLE 15

| | Order | Unigene | P | | Test | Cancer | |
|---|---|---|---|---|---|---|---|
| Rank | num | ID | value | FDR | AUC | cor | Gene description |
| 1 | 3059 | Hs.771 | <0.1 | <0.01 | 0.96 | −1 | gb: NM_002863.1 /DEF = Homo sapiens |
| | | | | | | | phosphorylase, /UG = Hs.771 phosphorylase, |
| | | | | | | | glycogen; liver |
| 2 | 13862 | Hs.66744 | <0.1 | <0.01 | 0.96 | 1 | Consensus includes |
| | | | | | | | gb: X99268.1/DEF = H./FL = gb: NM_000474.1 |
| 3 | 13045 | Hs.173094 | <0.1 | <0.01 | 1 | −1 | Consensus includes gb: AI096375/FEA = EST |
| 4 | 5759 | Hs.66052 | <0.1 | <0.01 | 0.97 | −1 | gb: NM_001775.1/DEF = Homo sapiens CD38 |
| 5 | 18621 | Hs.42824 | <0.1 | <0.01 | 0.95 | −1 | gb: NM_018192.1/DEF = Homo sapiens |
| | | | | | | | hypothetical |
| 6 | 3391 | Hs.139851 | <0.1 | <0.01 | 0.94 | −1 | gb: NM_001233.1/DEF = Homo sapiens caveolin |
| 7 | 18304 | Hs.34045 | <0.1 | <0.01 | 0.95 | 1 | gb: NM_017955.1/DEF = Homo sapiens |
| | | | | | | | hypothetical |
| 8 | 14532 | Hs.37035 | <0.1 | <0.01 | 1 | 1 | Consensus includes gb: AI738662/FEA = EST |
| 9 | 3577 | Hs.285754 | 0.1 | 0.01 | 1 | −1 | Consensus includes gb: BG170541/FEA = EST |
| 10 | 9010 | Hs.180446 | 0.1 | 0.01 | 1 | 1 | gb: L38951.1/DEF = Homo sapiens importin |
| 11 | 13497 | Hs.71465 | 0.1 | 0.01 | 1 | −1 | Consensus includes gb: AA639705/FEA = EST |
| 12 | 19488 | Hs.17752 | 0.1 | 0.01 | 1 | 1 | gb: NM_015900.1/DEF = Homo sapiens phosph |
| | | | | | | | phospholipase A1alpha/FL = gb: AF035268.1 |
| 13 | 8838 | Hs.237825 | 0.1 | 0.01 | 1 | 1 | gb: AF069765.1/DEF = Homo sapiens signal |
| | | | | | | | gb: NM_006947.1 |
| 14 | 14347 | Hs.170250 | 0.1 | 0.01 | 1 | 1 | Consensus includes gb: K02403.1/DEF = Human |
| 15 | 2300 | Hs.69469 | 0.2 | 0.01 | 1 | 1 | gb: NM_006360.1/DEF = Homo sapiens dendritic |
| 16 | 10973 | Hs.77899 | 0.2 | 0.01 | 1 | −1 | gb: Z24727.1/DEF = H. sapiens tropomyosin |
| 17 | 11073 | Hs.0 | 0.2 | 0.01 | 1 | 1 | gb: Z25434.1/DEF = H. sapiens protein- |
| | | | | | | | serinethreonine |
| 18 | 22193 | Hs.165337 | 0.2 | 0.01 | 1 | −1 | Consensus includes gb: AW971415/FE |
| 19 | 12742 | Hs.237506 | 0.2 | 0.01 | 1 | −1 | Consensus includes gb: AK023253.1/DEF= |
| 20 | 21823 | Hs.9614 | 0.3 | 0.01 | 1 | 1 | Consensus includes gb: AA191576/FEA = EST |
| 21 | 13376 | Hs.246885 | 0.3 | 0.01 | 1 | −1 | Consensus includes gb: W87466/FEA = EST |
| 22 | 6182 | Hs.77899 | 0.3 | 0.01 | 1 | −1 | gb: NM_000366.1/DEF = Homo sapiens |
| | | | | | | | tropomyosin |
| 23 | 3999 | Hs.1162 | 0.4 | 0.02 | 1 | 1 | gb: NM_002118.1/DEF = Homo sapiens major II, |
| | | | | | | | DM beta/FL = gb: NM_002118.1 gb: U15085.1 |
| 24 | 1776 | Hs.168670 | 0.7 | 0.03 | 1 | −1 | gb: NM_002857.1/DEF = Homo sapiens |
| | | | | | | | peroxisomal gb: AB018541.1 |
| 25 | 4046 | Hs.82568 | 0.7 | 0.03 | 1 | −1 | gb: NM_000784.1/DEF = Homo sapiens cytochrome |
| | | | | | | | cerebrotendinous xanthomatosis), polypeptide |
| 26 | 6924 | Hs.820 | 0.8 | 0.03 | 1 | 1 | gb: NM_004503.1/DEF = Homo sapiens homeo |
| 27 | 2957 | Hs.1239 | 0.9 | 0.03 | 1 | −1 | gb: NM_001150.1/DEF = Homo sapiens |
| | | | | | | | alanyl/DB_XREF = gi: 4502094/UG = Hs.1239 |
| | | | | | | | alanyl |
| 28 | 5699 | Hs.78406 | 1.3 | 0.05 | 1 | −1 | gb: NM_003558.1/DEF = Homo sapiens |
| | | | | | | | phosphatidylinositol phosphate 5-kinase, type I, |
| | | | | | | | beta/FL = gb: NM |
| 29 | 19167 | Hs.9238 | 1.4 | 0.05 | 1 | −1 | gb: NM_024539.1/DEF = Homo sapiens |
| | | | | | | | hypothetical |
| 30 | 4012 | Hs.172851 | 1.4 | 0.05 | 1 | −1 | gb: NM_001172.2/DEF = Homo sapiens arginase, |
| | | | | | | | gb: D86724.1 gb: U75667.1 gb: U82256.1 |
| 31 | 9032 | Hs.80658 | 1.4 | 0.05 | 1 | −1 | gb: U94592.1/DEF = Human uncoupling protein |
| | | | | | | | gb: U82819.1 gb: U94592.1 |
| 32 | 15425 | Hs.20141 | 1.5 | 0.05 | 1 | 1 | Consensus includes gb: AK000970.1/DEF= |
| 33 | 14359 | Hs.155956 | 1.6 | 0.05 | 1 | −1 | Consensus includes |
| | | | | | | | gb: NM_000662.1/DEF = acetyltransferase)/FL = gb: |
| | | | | | | | NM_000662.1 |
| 34 | 6571 | Hs.89691 | 1.6 | 0.05 | 1 | 1 | gb: NM_021139.1/DEF = Homo sapiens UDP |
| | | | | | | | polypeptide B4/FL = gb: NM_021139.1 |
| | | | | | | | gb: AF064200.1 |
| 35 | 13201 | Hs.301552 | 1.8 | 0.05 | 1 | 1 | Consensus includes gb: AK000478.1/DEF= |
| 36 | 21754 | Hs.292911 | 1.8 | 0.05 | 1 | −1 | Consensus includes gb: AI378979/FEA = EST |
| 37 | 5227 | Hs.31034 | 2 | 0.05 | 1 | −1 | Consensus includes gb: AL360141.1/DEF= |
| 38 | 18969 | Hs.20814 | 2.1 | 0.06 | 1 | 1 | gb: NM_015955.1/DEF = Homo sapiens CGI |
| 39 | 17907 | Hs.24395 | 2.2 | 0.06 | 1 | 1 | gb: NM_004887.1/DEF = Homo sapiens small small |
| | | | | | | | inducible cytokine subfamily B (Cys |
| 40 | 3831 | Hs.77695 | 2.3 | 0.06 | 1 | 1 | gb: NM_014750.1/DEF = Homo sapiens KIAA0008 |
| 41 | 10519 | Hs.4975 | 2.4 | 0.06 | 0.98 | 1 | gb: D82346.1/DEF = Homo sapiens mRNA |
| 42 | 2090 | Hs.150580 | 2.4 | 0.06 | 0.97 | −1 | gb: AF083441.1/DEF = Homo sapiens SUI1 |
| 43 | 9345 | Hs.75244 | 2.6 | 0.06 | 0.97 | −1 | gb: D87461.1/DEF = Human mRNA for KIAA0271 |
| 44 | 3822 | Hs.36708 | 2.7 | 0.06 | 0.97 | 1 | gb: NM_001211.2/DEF = Homo sapiens budding |
| | | | | | | | uninhibited by benzimidazoles 1 (yeast homolog) |
| 45 | 17999 | Hs.179666 | 2.9 | 0.06 | 0.97 | −1 | gb: NM_018478.1/DEF = Homo sapiens |
| | | | | | | | uncharacterized HSMNP1/FL = gb: BC001105.1 |
| | | | | | | | gb: AF220191.1 |
| 46 | 5070 | Hs.118140 | 2.9 | 0.06 | 0.96 | 1 | gb: NM_014705.1/DEF = Homo sapiens KIAA0716 |
| 47 | 20627 | Hs.288462 | 3 | 0.06 | 0.98 | −1 | gb: NM_025087.1/DEF = Homo sapiens |
| | | | | | | | hypothetical |
| 48 | 14690 | Hs.110826 | 3 | 0.06 | 0.99 | 1 | Consensus includes gb: AK027006.1/DEF= |
| 49 | 18137 | Hs.9641 | 3 | 0.06 | 0.98 | 1 | gb: NM_015991.1/DEF = Homo sapiens |
| | | | | | | | complement component 1, q subcomponent, alpha |
| | | | | | | | polypeptide-1 |
| 50 | 9594 | Hs.182278 | 3 | 0.06 | 0.98 | −1 | gb: BC000454.1/DEF = Homo sapiens, |
| | | | | | | | cal/FL = gb: BC000454.1 |

From Table 15, the combined criteria give an AUC of 1 between 8 and 40 genes. This indicates that subsets of up to 40 genes taken in the order of the criteria have a high predictive power. However, genes individually can also be judged for their predictive power by estimating p values. P values provide the probability that a gene is a random meaningless gene. A threshold can be set on that p value, e.g. 0.05.

Using the Bonferroni correction ensures that p values are not underestimated when a large number of genes are tested. This correction penalizes p values in proportion to the number of genes tested. Using 10*N probes (N=number of genes) the number of genes that score higher than all probes are significant at the threshold 0.1. Eight such genes were found with the combined criterion, while 26 genes were found with a p value<1.

It may be useful to filter out as many genes as possible before ranking them in order to avoid an excessive penalty. When the genes were filtered with the criterion that the standard deviation should exceed twice the mean (a criterion not involving any knowledge of how useful this gene is to predict cancer). This reduced the gene set to N′=571, but there were also only 8 genes at the significance level of 0.1 and 22 genes had p value<1.

The 8 first genes found by this method are given in Table 16. Genes over-expressed in cancer are under Rank 2, 7, and 8 (underlined). The remaining genes are under-expressed.

TABLE 16

| Rank | Unigene ID | Description and findings |
|---|---|---|
| 1 | Hs.771 | Phosphorylase, glycogen; liver (Hers disease, |
| | | glycogen storage disease type VI) (PYGL). |
| 2 | Hs.66744 | B-HLH DNA binding protein. H-twist. |
| 3 | Hs.173094 | KIAA1750 |
| 4 | Hs.66052 | CD38 antigen (p45) |
| 5 | Hs.42824 | FLJ10718 hypothetical protein |
| 6 | Hs.139851 | Caveolin 2 (CAV2) |
| 7 | Hs.34045 | FLJ20764 hypothetical protein |
| 8 | Hs.37035 | Homeo box HB9 |

Genes were ranked using the Pearson correlation criterion, see Table 17, with disease progression coded as Normal=1, Dysplasia=2, Grade3=3, Grade4=4. The p values are smaller than in the genes of Table 15, but the AUCs are worse. Three Febbo genes were found, corresponding to genes ranked 6^{th}, 7^{th} and 34^{th}.

TABLE 17

| | Order | | | | Test | Cancer | | |
|---|---|---|---|---|---|---|---|---|
| Rank | num | Unigene ID | Pvalue | FDR | AUC | cor | Febbo | Gene description |
| 1 | 6519 | Hs.243960 | <0.1 | <0.0003 | 0.85 | −1 | 0 | gb: NM_016250.1/DEF = Homo s |
| 2 | 9457 | Hs.128749 | <0.1 | <0.0003 | 0.93 | 1 | 0 | Consensus includes gb: AI796120 |
| 3 | 9976 | Hs.103665 | <0.1 | <0.0003 | 0.89 | −1 | 0 | gb: BC004300.1/DEF = Homo sapiens, |
| 4 | 9459 | Hs.128749 | <0.1 | <0.0003 | 0.87 | 1 | 0 | gb: AF047020.1/DEF = Homo sapiens |
| | | | | | | | | gb: NM_014324.1 |
| 5 | 9458 | Hs.128749 | <0.1 | <0.0003 | 0.89 | 1 | 0 | Consensus includes gb: AA888 |
| 6 | 12337 | Hs.7780 | <0.1 | <0.0003 | 0.96 | 1 | 1 | Consensus includes gb: AV715767 |
| 7 | 893 | Hs.226795 | <0.1 | <0.0003 | 0.97 | −1 | 1 | gb: NM_000852.2/DEF = Homo sapiens |
| 8 | 19589 | Hs.45140 | <0.1 | <0.0003 | 0.98 | −1 | 0 | gb: NM_021637.1/DEF = Homo sapiens |
| 9 | 11911 | Hs.279009 | <0.1 | <0.0003 | 0.98 | −1 | 0 | Consensus includes gb: AI653730 |
| 10 | 17944 | Hs.279905 | <0.1 | <0.0003 | 0.96 | 1 | 0 | gb: NM_016359.1/DEF = Homo sapiens |
| | | | | | | | | gb: AF290612.1 gb: AF090915.1 |
| 11 | 9180 | Hs.239926 | <0.1 | <0.0003 | 0.96 | −1 | 0 | Consensus includes gb: AV704962 |
| 12 | 18122 | Hs.106747 | <0.1 | <0.0003 | 0.96 | −1 | 0 | gb: NM_021626.1/DEF = Homo sapiens |
| | | | | | | | | protein /FL = gb: AF282618.1 gb: NM_{—} |
| 13 | 12023 | Hs.74034 | <0.1 | <0.0003 | 0.96 | −1 | 0 | Consensus includes gb: AU14739 |
| 14 | 374 | Hs.234642 | <0.1 | <0.0003 | 0.96 | −1 | 0 | Cluster Incl. 74607: za55a01.s1 |
| 15 | 12435 | Hs.82432 | <0.1 | <0.0003 | 0.96 | −1 | 0 | Consensus includes b: AA135522 |
| 16 | 18598 | Hs.9728 | <0.1 | <0.0003 | 0.96 | −1 | 0 | gb: NM_016608.1/DEF = Homo sapiens |
| 17 | 3638 | Hs.74120 | <0.1 | <0.0003 | 0.97 | −1 | 0 | gb: NM_006829.1/DEF = Homo sapiens |
| 18 | 5150 | Hs.174151 | <0.1 | <0.0003 | 0.97 | −1 | 0 | gb: NM_001159.2/DEF = Homo sapiens |
| 19 | 1889 | Hs.195850 | <0.1 | <0.0003 | 0.97 | −1 | 0 | gb: NM_000424.1/DEF = Homo |
| | | | | | | | | sapiens/DB_XREF = gi: 4557889/UG = Hs. |
| 20 | 3425 | Hs.77256 | <0.1 | <0.0003 | 0.97 | 1 | 0 | gb: NM_004456.1/DEF = Homo |
| | | | | | | | | sapiens/FL = gb: U61145.1 |
| | | | | | | | | gb: NM_004456.1 |
| 21 | 5149 | Hs.174151 | <0.1 | <0.0003 | 0.96 | −1 | 0 | gb: AB046692.1/DEF = Homo sapiens |
| 22 | 4351 | Hs.303090 | <0.1 | <0.0003 | 0.97 | −1 | 0 | Consensus includes gb: N26005 |
| 23 | 4467 | Hs.24587 | <0.1 | <0.0003 | 0.97 | −1 | 0 | gb: NM_005864.1/DEF = Homo |
| | | | | | | | | sapiens/FL = gb: AB001466.1 |
| | | | | | | | | gb: NM_005864.1 |
| 24 | 12434 | Hs.250723 | <0.1 | <0.0003 | 0.96 | −1 | 0 | Consensus includes gb: BF968134 |
| 25 | 12809 | Hs.169401 | <0.1 | <0.0003 | 0.95 | 1 | 0 | Consensus includes gb: AI358867 |
| 26 | 7082 | Hs.95197 | <0.1 | <0.0003 | 0.95 | −1 | 0 | gb: AB015228.1/DEF = Homo sapiens |
| | | | | | | | | gb: AB015228.1 |
| 27 | 18659 | Hs.73625 | <0.1 | <0.0003 | 0.95 | 1 | 0 | gb: NM_005733.1/DEF = Homo sapiens |
| | | | | | | | | (rabkinesin6)/FL = gb: AF070672.1 |
| 28 | 13862 | Hs.66744 | <0.1 | <0.0003 | 0.98 | 1 | 0 | Consensus includes gb: X99268.1 |
| | | | | | | | | syndrome)/FL = gb: NM_000474 |
| 29 | 3059 | Hs.771 | <0.1 | <0.0003 | 0.98 | −1 | 0 | gb: NM_002863.1/DEF = Homo |
| | | | | | | | | sapiens/DB_XREF = gi: 4506352/UG = Hs. |
| 30 | 15294 | Hs.288649 | <0.1 | <0.0003 | 0.98 | 1 | 0 | Consensus includes gb: AK0 |
| 31 | 9325 | Hs.34853 | <0.1 | <0.0003 | 0.99 | −1 | 0 | Consensus includes gb: AW157094 |
| 32 | 18969 | Hs.20814 | <0.1 | <0.0003 | 0.98 | 1 | 0 | gb: NM_015955.1/DEF = Homo sapiens |
| 33 | 4524 | Hs.65029 | <0.1 | <0.0003 | 0.96 | −1 | 0 | gb: NM_002048.1/DEF = Homo sapiens |
| 34 | 1908 | Hs.692 | <0.1 | <0.0003 | 0.97 | 1 | 1 | gb: NM_002354.1/DEF = Homo sapiens |
| | | | | | | | | signal transducer 1/FL = gb: M32306.1 |
| 35 | 11407 | Hs.326776 | <0.1 | <0.0003 | 0.96 | −1 | 0 | gb: AF180519.1/DEF = Homo sapiens |
| | | | | | | | | cds/FL = gb: AF180519.1 |
| 36 | 19501 | Hs.272813 | <0.1 | <0.0003 | 0.96 | −1 | 0 | gb: NM_017434.1/DEF = Homo sapiens |
| 37 | 11248 | Hs.17481 | <0.1 | <0.0003 | 0.96 | −1 | 0 | gb: AF063606.1/DEF = Homo sapiens |
| 38 | 5894 | Hs.80247 | <0.1 | <0.0003 | 0.95 | −1 | 0 | gb: NM_000729.2/DEF = Homo sapiens |
| 39 | 19455 | Hs.26892 | <0.1 | <0.0003 | 0.96 | −1 | 0 | gb: NM_018456.1/DEF = Homo sapie |
| | | | | | | | | BM040/FL = gb: AF217516.1 gb: |
| 40 | 3448 | Hs.169401 | <0.1 | <0.0003 | 0.96 | 1 | 0 | Consensus includes gb: N33009 |
| 41 | 6666 | Hs.90911 | <0.1 | <0.0003 | 0.96 | −1 | 0 | gb: NM_004695.1/DEF = Homo |
| | | | | | | | | sapiens/UG = Hs.90911 solute carrier |
| | | | | | | | | family |
| 42 | 6924 | Hs.820 | <0.1 | <0.0003 | 0.98 | 1 | 0 | gb: NM_004503.1/DEF = Homo sapiens |
| 43 | 2169 | Hs.250811 | <0.1 | <0.0003 | 0.98 | −1 | 0 | Consensus includes gb: BG169673 |
| 44 | 12168 | Hs.75318 | <0.1 | <0.0003 | 0.98 | −1 | 0 | Consensus includes gb: AL565074 |
| 45 | 18237 | Hs.283719 | <0.1 | <0.0003 | 0.98 | −1 | 0 | gb: NM_018476.1/DEF = Homo sapiens |
| | | | | | | | | HBEX2/FL = gb: AF220189.1 gb: |
| 46 | 5383 | Hs.182575 | <0.1 | <0.0003 | 0.98 | −1 | 0 | Consensus includes gb: BF223679 |
| 47 | 19449 | Hs.17296 | <0.1 | <0.0003 | 0.99 | −1 | 0 | gb: NM_023930.1/DEF = Homo sapiens |
| | | | | | | | | gb: BC001929.1 gb: NM_023930.1 |
| 48 | 4860 | Hs.113082 | <0.1 | <0.0003 | 0.99 | −1 | 0 | gb: NM_014710.1/DEF = Homo sapiens |
| 49 | 17714 | Hs.5216 | <0.1 | <0.0003 | 0.99 | 1 | 0 | gb: NM_014038.1/DEF = Homo sapiens |
| 50 | 12020 | Hs.137476 | <0.1 | <0.0003 | 0.97 | −1 | 0 | Consensus includes gb: AL582836 |

The data is rich in potential biomarkers. To find the most promising markers, criteria were designed to implement prior knowledge of disease severity and zonal information. This allowed better separation of relevant genes from genes that coincidentally well separate the data, thus alleviating the problem of overfitting. To further reduce the risk of overfitting, genes were selected that were also found in an independent study Table 15. Those genes include well-known proteins involved in prostate cancer and some potentially interesting targets.

### Example 5

### Prostate Cancer Gene Expression Microarray Data (11-2004)

Several separations of class pairs were performed including “BPH vs. non-BPH” and “tumor (G3+4) vs. all other tissues”. These separations are relatively easy and can be performed with fewer than 10 genes, however, hundreds of significant genes were identified. The best AUCs (Area under the ROC curve) and BER (balanced error rate) in 10×10-fold cross-validation experiments are on the order of AUC_{BPH}=0.995, BER_{BPH}=5%, AUC_{G34}=0.94, BER_{G34}=9%.

Separations of “G4 vs. all others”, “Dysplasia vs. all others”, and “Normal vs. all others” are less easy (best AUCs between 0.75 and 0.85) and separation of “G3 vs. all others” is almost impossible in this data (AUC around 0.5). With over 100 genes, G4 can be separated from all other tissues with about 10% BER. Hundreds of genes separate G4 from all other tissues significantly, yet one cannot find a good separation with just a few genes.

Separations of “TZG4 vs. PZG4”, “Normal vs. Dysplasia” and “G3 vs. G4” are also hard. 10×10-fold CV yielded very poor results. Using leave-one out CV and under 20 genes, we separated some pairs of classes: ERR_{TZG4/PZG4}≈6%, ERR_{NL/Dys} and ERR_{G3/G4}≈9%. However, due to the small sample sizes, the significance of the genes found for those separations is not good, shedding doubt on the results.

Pre-operative PSA was found to correlate poorly with clinical variables (R²=0.316 with cancer volume, 0.025 with prostate weight, and 0.323 with CAvol/Weight). Genes were found with activity that correlated with pre-operative PSA either in BPH samples or G34 samples or both. Possible connections of those genes were found to cancer and/or prostate in the literature, but their relationship to PSA is not documented. Genes associated to PSA by their description do not have expression values correlated with pre-operative PSA. This illustrates that gene expression coefficients do not necessarily reflect the corresponding protein abundance.

Genes were identified that correlate with cancer volume in G3+4 tissues and with cure/fail prognosis. Neither are statistically significant, however, the gene most correlated with cancer volume has been reported in the literature as connected to prostate cancer. Prognosis information can be used in conjunction with grade levels to determine the significance of genes. Several genes were identified for separating G4 from non-G4 and G3 from G3 in the group the samples of patients with the poor prognosis in regions of lowest expression values.

The following experiments were performed using data consisting of a matrix of 87 lines (samples) and 22283 columns (genes) obtained from an Affymetrix U133A GeneChip®. The distributions of the samples of the microarray prostate cancer study are the same as those listed in Table 12.

Genes were selected on the basis of their individual separating power, as measured by the AUC (area under the ROC curve that plots sensitivity vs. specificity).

Similarly “random genes” that are genes obtained by permuting randomly the values of columns of the matrix are ranked. Where N is the total number of genes (here, N=22283, 40 times more random genes than real genes are used to estimate p values accurately (Nᵣ=40*22283). For a given AUC value A, nᵣ(A) is the number of random genes that have an AUC larger than A. The p value is estimated by the fraction of random genes that have an AUC larger than A, i.e.:

Pvalue=(1+nᵣ(A))/Nᵣ

Adding 1 to the numerator avoids having zero p values for the best ranking genes and accounts for the limited precision due to the limited number of random genes. Because the pvalues of a large number of genes are measured simultaneously, correction must be applied to account for this multiple testing. As in the previous example, the simple Bonferroni correction is used:

Bonferroni_{—}pvalue=N*(1+nᵣ(A))/Nᵣ

Hence, with a number of probes that is 40 times the number of genes, the p values are estimated with an accuracy of 0.025.

For a given gene of AUC value A, one can also compute the false discovery rate (FDR), which is an estimate of the ratio of the number of falsely significant genes over the number of genes called significant. Where n(A) is the number of genes found above A, the FDR is computed as the ratio of the p value (before Bonferroni correction) and the fraction of real genes found above A:

FDR=pvalue*N/n(A)=((1+nᵣ(A))*N)/(n(A)*Nᵣ).

Linear ridge regression classifiers (similar to SVMs) were trained with 10×10-fold cross validation, i.e., the data were split 100 times into a training set and a test set and the average performance and standard deviation were computed. In these experiments, the feature selection is performed within the cross-validation loop. That is, a separate featuring ranking is performed for each data split. The number of features are varied and a separate training/testing is performed for each number of features. Performances for each number of features are averaged to plot performance vs. number of features. The ridge value is optimized separately for each training subset and number of features, using the leave-one-out error, which can be computed analytically from the training error. In some experiments, the 10×10-fold cross-validation was done by leave-one-out cross-validation. Everything else remains the same.

Using the rankings obtained for the 100 data splits of the machine learning experiments (also called “bootstraps”), average gene ranks are computed. Average gene rank carries more information in proportion to the fraction of time a gene was always found in the top N ranking genes. This last criterion is sometimes used in the literature, but the number of genes always found in the top N ranking genes appears to grows linearly with N.

The following statistics were computed for cross-validation (10 times 10-fold or leave-one-out) of the machine learning experiments:

AUC mean: The average area under the ROC curve over all data splits.

AUC stdev: The corresponding standard deviation. Note that the standard error obtained by dividing stdev by the square root of the number of data splits is inaccurate because sampling is done with replacements and the experiments are not independent of one another.

BER mean: The average BER over all data splits. The BER is the balanced error rate, which is the average of the error rate of examples of the first class and examples of the second class. This provides a measure that is not biased toward the most abundant class.

BER stdev: The corresponding standard deviation.

Pooled AUC: The AUC obtained using the predicted classification values of all the test examples in all data splits altogether.

Pooled BER: The BER obtained using the predicted classification values of all the test examples in all data splits altogether.

Note that for leave-one-out CV, it does not make sense to compute BER-mean because there is only one example in each test set. Instead, the leave-one-out error rate or the pooled BER is computed.

The first set of experiments was directed to the separation BPH vs. all others. In previous reports, genes were found to be characteristic of BPH, e.g., gene 3480 (Hs.79389, NELL2).

Of the top 100 genes separating best BPH from all other samples, a very clear separation is found, even with only two genes. In these experiments, gene complementarity was not sought. Rather, genes were selected for their individual separating power. The top two genes are the same as those described in Example 4: gene 3480 (NELL2) and gene 5309 (SCYB13).

Table 17 provides the results of the machine learning experiments for BPH vs. non BPH separation with varying number of features, in the range 2-16 features.

TABLE 17

| | Feat. num. | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 16 | 32 | 64 | 128 |
| 100 * | 98.5 | 99.63 | 99.75 | 99.75 | 99.63 | 99.63 | 99.63 | 99.63 | 99.75 | 99.63 | 99.63 | 99.25 | 96.6 | 92.98 |
| AUC | | | | | | | | | | | | | | |
| 100 * | 4.79 | 2.14 | 1.76 | 1.76 | 2.14 | 2.14 | 2.14 | 2.14 | 1.76 | 2.14 | 2.14 | 3.47 | 10.79 | 17.43 |
| AUC | | | | | | | | | | | | | | |
| std | | | | | | | | | | | | | | |
| BER | 9.75 | 5.06 | 5.31 | 5.06 | 5 | 5.19 | 5.31 | 5.31 | 5.31 | 5.44 | 5.19 | 5.85 | 7.23 | 18.66 |
| (%) | | | | | | | | | | | | | | |
| BERstd | 20.11 | 15.07 | 15.03 | 15.07 | 15.08 | 15.05 | 15.03 | 15.03 | 15.03 | 15.01 | 15.05 | 14.96 | 16.49 | 24.26 |
| (%) | | | | | | | | | | | | | | |

Very high classification accuracy (as measured by the AUC) is achieved with only 2 genes to provide the AUC above 0.995. The error rate and the AUC are mostly governed by the outlier and the balanced error rate (BER) below 5.44%. Also included is the standard deviation of the 10×10-fold experiment. If the experimental repeats were independent, the standard error of the mean obtained by dividing the standard deviation by 10 could be used as error bar. A more reasonable estimate of the error bar may be obtained by dividing it by three to account for the dependencies between repeats, yielding an error bar of 0.006 for the best AUCs and 5% for BER. For the best AUCs, the error is essentially due to one outlier (1.2% error and 5% balanced error rate). The list of the top 100 genes separating BPH from other tissues is given in Table 18.

TABLE 18

| | | | Under | | | | |
|---|---|---|---|---|---|---|---|
| | Gene | Unigene | Expr. | | | | Ave. |
| Rank | ID | ID | In BPH | AUC | Pval | FDR | rank |
| 1 | 5309 | Hs.100431 | −1 | 0.9974 | 0.02 | 0.025 | 1.39 |
| 2 | 3480 | Hs.79389 | −1 | 0.9948 | 0.02 | 0.012 | 2.13 |
| 3 | 5810 | Hs.56045 | −1 | 0.9922 | 0.02 | 0.0083 | 3.43 |
| 4 | 3063 | Hs.79732 | −1 | 0.9896 | 0.02 | 0.0062 | 4.27 |
| 5 | 17802 | Hs.3807 | −1 | 0.9844 | 0.02 | 0.005 | 5.85 |
| 6 | 5497 | Hs.1104 | −1 | 0.9792 | 0.02 | 0.0042 | 7.61 |
| 7 | 19651 | Hs.16026 | −1 | 0.9779 | 0.02 | 0.0036 | 9.59 |
| 8 | 5715 | Hs.89791 | −1 | 0.9766 | 0.02 | 0.0031 | 10.34 |
| 9 | 4843 | Hs.75774 | 1 | 0.974 | 0.02 | 0.0028 | 11.62 |
| 10 | 5498 | Hs.1104 | −1 | 0.974 | 0.02 | 0.0025 | 12.03 |
| 11 | 11301 | Hs.211933 | −1 | 0.974 | 0.02 | 0.0023 | 13.11 |
| 12 | 1217 | Hs.245188 | −1 | 0.9727 | 0.02 | 0.0021 | 12.08 |
| 13 | 3490 | Hs.101850 | −1 | 0.9714 | 0.02 | 0.0019 | 14.93 |
| 14 | 5631 | Hs.95420 | −1 | 0.9701 | 0.02 | 0.0018 | 15.38 |
| 15 | 5449 | Hs.155597 | −1 | 0.9675 | 0.02 | 0.0017 | 16.93 |
| 16 | 3254 | Hs.81256 | −1 | 0.9662 | 0.02 | 0.0016 | 17.68 |
| 17 | 6443 | Hs.44481 | −1 | 0.9662 | 0.02 | 0.0015 | 19.16 |
| 18 | 4779 | Hs.284122 | −1 | 0.9597 | 0.02 | 0.0014 | 26.7 |
| 19 | 19044 | Hs.76461 | −1 | 0.9597 | 0.02 | 0.0013 | 26.26 |
| 20 | 9201 | Hs.5422 | −1 | 0.9584 | 0.02 | 0.0012 | 29.52 |
| 21 | 9469 | Hs.5378 | −1 | 0.9584 | 0.02 | 0.0012 | 28.97 |
| 22 | 1216 | Hs.245188 | −1 | 0.9571 | 0.02 | 0.0011 | 28.79 |
| 23 | 4078 | Hs.18676 | −1 | 0.9571 | 0.02 | 0.0011 | 28.55 |
| 24 | 9897 | Hs.26468 | −1 | 0.9565 | 0.02 | 0.001 | 30.78 |
| 25 | 3416 | Hs.43697 | −1 | 0.9558 | 0.02 | 0.001 | 32.51 |
| 26 | 19841 | Hs.6510 | −1 | 0.9558 | 0.02 | 0.00096 | 33.54 |
| 27 | 1219 | Hs.245188 | −1 | 0.9545 | 0.02 | 0.00093 | 32.37 |
| 28 | 9713 | Hs.77202 | −1 | 0.9545 | 0.02 | 0.00089 | 35.5 |
| 29 | 20879 | Hs.0 | −1 | 0.9545 | 0.02 | 0.00086 | 34.66 |
| 30 | 1856 | Hs.79732 | −1 | 0.9532 | 0.02 | 0.00083 | 34.47 |
| 31 | 2037 | Hs.1869 | −1 | 0.9532 | 0.02 | 0.00081 | 34.78 |
| 32 | 3970 | Hs.31720 | −1 | 0.9532 | 0.02 | 0.00078 | 36.27 |
| 33 | 18622 | Hs.43080 | −1 | 0.9519 | 0.02 | 0.00076 | 40.94 |
| 34 | 3311 | Hs.154103 | 1 | 0.9506 | 0.02 | 0.00074 | 40.34 |
| 35 | 3399 | Hs.155939 | −1 | 0.9506 | 0.02 | 0.00071 | 40.66 |
| 36 | 5022 | Hs.15154 | −1 | 0.9506 | 0.02 | 0.00069 | 40.82 |
| 37 | 12549 | Hs.169965 | −1 | 0.9506 | 0.02 | 0.00068 | 42.46 |
| 38 | 4998 | Hs.78061 | −1 | 0.9494 | 0.02 | 0.00066 | 43.74 |
| 39 | 9574 | Hs.85112 | −1 | 0.9494 | 0.02 | 0.00064 | 44.58 |
| 40 | 13062 | Hs.93005 | −1 | 0.9494 | 0.02 | 0.00062 | 44.03 |
| 41 | 16714 | Hs.306913 | 1 | 0.9481 | 0.02 | 0.00061 | 46.98 |
| 42 | 4467 | Hs.24587 | −1 | 0.9468 | 0.02 | 0.0006 | 48.29 |
| 43 | 6001 | Hs.153322 | −1 | 0.9468 | 0.02 | 0.00058 | 49.03 |
| 44 | 20655 | Hs.10235 | −1 | 0.9468 | 0.02 | 0.00057 | 50.13 |
| 45 | 1055 | Hs.151242 | −1 | 0.9455 | 0.02 | 0.00056 | 49.6 |
| 46 | 5819 | Hs.75652 | −1 | 0.9455 | 0.02 | 0.00054 | 48.21 |
| 47 | 11595 | Hs.0 | −1 | 0.9455 | 0.02 | 0.00053 | 49.96 |
| 48 | 1911 | Hs.76224 | −1 | 0.9442 | 0.02 | 0.00052 | 53.85 |
| 49 | 6136 | Hs.123642 | −1 | 0.9442 | 0.02 | 0.00051 | 54.24 |
| 50 | 19274 | Hs.100890 | −1 | 0.9416 | 0.02 | 0.0005 | 56.43 |
| 51 | 20091 | Hs.44208 | 1 | 0.9416 | 0.02 | 0.00049 | 57.99 |
| 52 | 5195 | Hs.88474 | −1 | 0.9403 | 0.02 | 0.00048 | 61.54 |
| 53 | 5431 | Hs.9795 | −1 | 0.9403 | 0.02 | 0.00047 | 62.04 |
| 54 | 15456 | Hs.25220 | −1 | 0.9403 | 0.02 | 0.00046 | 61.16 |
| 55 | 3484 | Hs.83551 | −1 | 0.939 | 0.02 | 0.00045 | 61.34 |
| 56 | 14516 | Hs.162209 | 1 | 0.939 | 0.02 | 0.00045 | 60.79 |
| 57 | 18728 | Hs.8395 | −1 | 0.939 | 0.02 | 0.00044 | 63.14 |
| 58 | 12337 | Hs.7780 | 1 | 0.9377 | 0.02 | 0.00043 | 63.72 |
| 59 | 3392 | Hs.146428 | −1 | 0.937 | 0.02 | 0.00042 | 63.99 |
| 60 | 8440 | Hs.1408 | −1 | 0.9364 | 0.02 | 0.00042 | 65.71 |
| 61 | 9322 | Hs.260024 | −1 | 0.9351 | 0.02 | 0.00041 | 69.4 |
| 62 | 12156 | Hs.173717 | −1 | 0.9351 | 0.02 | 0.0004 | 70.82 |
| 63 | 3061 | Hs.78065 | −1 | 0.9338 | 0.02 | 0.0004 | 73.88 |
| 64 | 10028 | Hs.50924 | −1 | 0.9338 | 0.02 | 0.00039 | 74.75 |
| 65 | 19331 | Hs.20914 | −1 | 0.9325 | 0.02 | 0.00038 | 79.34 |
| 66 | 1138 | Hs.111301 | −1 | 0.9318 | 0.02 | 0.00038 | 76.07 |
| 67 | 3310 | Hs.154103 | 1 | 0.9312 | 0.02 | 0.00037 | 78.77 |
| 68 | 19574 | Hs.26270 | −1 | 0.9312 | 0.02 | 0.00037 | 78.28 |
| 69 | 1000 | Hs.75350 | −1 | 0.9299 | 0.02 | 0.00036 | 81.37 |
| 70 | 18099 | Hs.7527 | 1 | 0.9299 | 0.02 | 0.00036 | 82.61 |
| 71 | 2756 | Hs.83429 | 1 | 0.9286 | 0.02 | 0.00035 | 86.12 |
| 72 | 5414 | Hs.56145 | 1 | 0.9286 | 0.02 | 0.00035 | 83.03 |
| 73 | 9715 | Hs.237356 | −1 | 0.9286 | 0.02 | 0.00034 | 84.98 |
| 74 | 12116 | Hs.21858 | −1 | 0.9286 | 0.02 | 0.00034 | 88.77 |
| 75 | 13913 | Hs.5378 | −1 | 0.9286 | 0.02 | 0.00033 | 88.67 |
| 76 | 17755 | Hs.279923 | 1 | 0.9286 | 0.02 | 0.00033 | 82.62 |
| 77 | 2020 | Hs.10247 | 1 | 0.9273 | 0.02 | 0.00032 | 83.71 |
| 78 | 3629 | Hs.79226 | −1 | 0.9273 | 0.02 | 0.00032 | 87.52 |
| 79 | 3686 | Hs.182859 | −1 | 0.9266 | 0.02 | 0.00032 | 85.78 |
| 80 | 9457 | Hs.128749 | 1 | 0.9266 | 0.02 | 0.00031 | 90.87 |
| 81 | 1646 | Hs.118638 | 1 | 0.926 | 0.02 | 0.00031 | 86.31 |
| 82 | 3064 | Hs.79732 | −1 | 0.926 | 0.02 | 0.0003 | 87.56 |
| 83 | 13911 | Hs.408 | −1 | 0.926 | 0.02 | 0.0003 | 91.63 |
| 84 | 1396 | Hs.82916 | 1 | 0.9247 | 0.02 | 0.0003 | 90.31 |
| 85 | 1912 | Hs.76224 | −1 | 0.9208 | 0.02 | 0.00029 | 105.84 |
| 86 | 9398 | Hs.81071 | −1 | 0.9208 | 0.02 | 0.00029 | 106.92 |
| 87 | 13823 | Hs.8077 | −1 | 0.9208 | 0.02 | 0.00029 | 104.9 |
| 88 | 20815 | Hs.288348 | −1 | 0.9208 | 0.02 | 0.00028 | 101.86 |
| 89 | 5451 | Hs.160318 | −1 | 0.9201 | 0.02 | 0.00028 | 100.98 |
| 90 | 2478 | Hs.251664 | −1 | 0.9195 | 0.02 | 0.00028 | 100.48 |
| 91 | 2989 | Hs.117970 | −1 | 0.9195 | 0.02 | 0.00027 | 108.72 |
| 92 | 11607 | Hs.0 | −1 | 0.9195 | 0.02 | 0.00027 | 104.67 |
| 93 | 8179 | Hs.0 | −1 | 0.9182 | 0.02 | 0.00027 | 104.03 |
| 94 | 11464 | Hs.68879 | −1 | 0.9182 | 0.02 | 0.00027 | 107.39 |
| 95 | 13321 | Hs.76536 | −1 | 0.9182 | 0.02 | 0.00026 | 110 |
| 96 | 9163 | Hs.119498 | −1 | 0.9169 | 0.02 | 0.00026 | 111.55 |
| 97 | 14166 | Hs.278503 | −1 | 0.9169 | 0.02 | 0.00026 | 112.58 |
| 98 | 1574 | Hs.82124 | −1 | 0.9156 | 0.02 | 0.00026 | 117.67 |
| 99 | 13211 | Hs.159263 | −1 | 0.9156 | 0.02 | 0.00025 | 116.28 |
| 100 | 20538 | Hs.143907 | −1 | 0.9156 | 0.02 | 0.00025 | 116 |

In Tables 18-37, genes are ranked by their individual AUC computed with all the data. The first column is the rank, followed by the Gene ID (order number in the data matrix), and the Unigene ID. The column “Under Expr” is +1 if the gene is underexpressed and −1 otherwise. AUC is the ranking criterion. Pval is the pvalue computed with random genes as explained above. FDR is the false discovery rate. “Ave. rank” is the average rank of the feature when subsamples of the data are taken in a 10×10-fold cross-validation experiment in Tables 18-28 and with leave-one-out in Tables 30-37.

A similar set of experiments was conducted to separate tumors (cancer G3 and G4) from other tissues. The results show that it is relatively easy to separate tumor from other tissues (although not as easy as separating the BPH). The list of the top 50 tumor genes is shown in Table 19. The three best genes, Gene IDs no. 9457, 9458 and 9459 all have same Unigene ID. Additional description is provided in Table 20 below.

TABLE 19

| | | | Under | | | | |
|---|---|---|---|---|---|---|---|
| | Gene | Unigene | Expr. | | | | Ave. |
| Rank | ID | ID | In tumor | AUC | Pval | FDR | rank |
| 1 | 9459 | Hs.128749 | −1 | 0.9458 | 0.02 | 0.025 | 1.16 |
| 2 | 9458 | Hs.128749 | −1 | 0.9425 | 0.02 | 0.012 | 2.48 |
| 3 | 9457 | Hs.128749 | −1 | 0.9423 | 0.02 | 0.0083 | 2.51 |
| 4 | 11911 | Hs.279009 | 1 | 0.9253 | 0.02 | 0.0062 | 4.31 |
| 5 | 12337 | Hs.7780 | −1 | 0.9125 | 0.02 | 0.005 | 7.23 |
| 6 | 983 | Hs.226795 | 1 | 0.9076 | 0.02 | 0.0042 | 8.42 |
| 7 | 18792 | Hs.6823 | −1 | 0.9047 | 0.02 | 0.0036 | 10.04 |
| 8 | 1908 | Hs.692 | −1 | 0.9044 | 0.02 | 0.0031 | 10.03 |
| 9 | 19589 | Hs.45140 | 1 | 0.9033 | 0.02 | 0.0028 | 10.47 |
| 10 | 6519 | Hs.243960 | 1 | 0.8996 | 0.02 | 0.0025 | 12.67 |
| 11 | 17714 | Hs.5216 | −1 | 0.8985 | 0.02 | 0.0023 | 13.93 |
| 12 | 18122 | Hs.106747 | 1 | 0.8985 | 0.02 | 0.0021 | 13.86 |
| 13 | 18237 | Hs.283719 | 1 | 0.8961 | 0.02 | 0.0019 | 16.61 |
| 14 | 3059 | Hs.771 | 1 | 0.8942 | 0.02 | 0.0018 | 17.86 |
| 15 | 16533 | Hs.110826 | −1 | 0.8921 | 0.02 | 0.0017 | 19.44 |
| 16 | 18598 | Hs.9728 | 1 | 0.8904 | 0.02 | 0.0016 | 19.43 |
| 17 | 12434 | Hs.250723 | 1 | 0.8899 | 0.02 | 0.0015 | 20.19 |
| 18 | 4922 | Hs.55279 | 1 | 0.884 | 0.02 | 0.0014 | 27.23 |
| 19 | 13862 | Hs.66744 | −1 | 0.8832 | 0.02 | 0.0013 | 30.59 |
| 20 | 9976 | Hs.103665 | 1 | 0.8824 | 0.02 | 0.0012 | 30.49 |
| 21 | 18835 | Hs.44278 | −1 | 0.8824 | 0.02 | 0.0012 | 30.94 |
| 22 | 3331 | Hs.54697 | 1 | 0.8802 | 0.02 | 0.0011 | 32.35 |
| 23 | 18969 | Hs.20814 | −1 | 0.8797 | 0.02 | 0.0011 | 35.89 |
| 24 | 9373 | Hs.21293 | −1 | 0.8786 | 0.02 | 0.001 | 35.52 |
| 25 | 15294 | Hs.288649 | −1 | 0.8786 | 0.02 | 0.001 | 35.69 |
| 26 | 4497 | Hs.33084 | 1 | 0.8776 | 0.02 | 0.00096 | 37.77 |
| 27 | 5001 | Hs.823 | −1 | 0.8765 | 0.02 | 0.00093 | 40.25 |
| 28 | 9765 | Hs.22599 | 1 | 0.8765 | 0.02 | 0.00089 | 39.32 |
| 29 | 4479 | Hs.198760 | 1 | 0.8759 | 0.02 | 0.00086 | 40.82 |
| 30 | 239 | Hs.198760 | 1 | 0.8749 | 0.02 | 0.00083 | 43.04 |
| 31 | 6666 | Hs.90911 | 1 | 0.8749 | 0.02 | 0.00081 | 42.53 |
| 32 | 12655 | Hs.10587 | 1 | 0.8749 | 0.02 | 0.00078 | 41.56 |
| 33 | 19264 | Hs.31608 | −1 | 0.8743 | 0.02 | 0.00076 | 44.66 |
| 34 | 5923 | Hs.171731 | 1 | 0.8738 | 0.02 | 0.00074 | 44.3 |
| 35 | 1889 | Hs.195850 | 1 | 0.8727 | 0.02 | 0.00071 | 46.1 |
| 36 | 21568 | Hs.111676 | 1 | 0.8716 | 0.02 | 0.00069 | 48.3 |
| 37 | 3264 | Hs.139336 | −1 | 0.8714 | 0.02 | 0.00068 | 51.17 |
| 38 | 14738 | Hs.8198 | 1 | 0.8706 | 0.02 | 0.00066 | 52.7 |
| 39 | 1867 | Hs.234680 | 1 | 0.8695 | 0.02 | 0.00064 | 52.99 |
| 40 | 4467 | Hs.24587 | 1 | 0.8695 | 0.02 | 0.00062 | 52.25 |
| 41 | 9614 | Hs.8583 | 1 | 0.8695 | 0.02 | 0.00061 | 53.62 |
| 42 | 18659 | Hs.73625 | −1 | 0.8692 | 0.02 | 0.0006 | 56.86 |
| 43 | 20137 | Hs.249727 | 1 | 0.8692 | 0.02 | 0.00058 | 55.2 |
| 44 | 12023 | Hs.74034 | 1 | 0.869 | 0.02 | 0.00057 | 55.69 |
| 45 | 12435 | Hs.82432 | 1 | 0.869 | 0.02 | 0.00056 | 56.63 |
| 46 | 14626 | Hs.23960 | −1 | 0.8687 | 0.02 | 0.00054 | 58.95 |
| 47 | 7082 | Hs.95197 | 1 | 0.8684 | 0.02 | 0.00053 | 56.27 |
| 48 | 15022 | Hs.110826 | −1 | 0.8679 | 0.02 | 0.00052 | 59.51 |
| 49 | 20922 | Hs.0 | −1 | 0.8679 | 0.02 | 0.00051 | 59.93 |
| 50 | 4361 | Hs.102 | 1 | 0.8673 | 0.02 | 0.0005 | 60.94 |

TABLE 20

| Gene ID | Description |
|---|---|
| 9457 | gb: AI796120/FEA = EST/DB_XREF = gi: 5361583/DB_XREF = est: wh42f03.x1/ |
| | CLONE = IMAGE: 2383421/UG = Hs.128749 alphamethylacyl-CoA racemase/ |
| | FL = gb: AF047020.1 gb: AF158378.1 gb: NM_014324.1 |
| 9458 | gb: AA888589/FEA = EST/DB_XREF = gi: 3004264/DB_XREF = est: oe68e10.s1/ |
| | CLONE = IMAGE: 1416810/UG = Hs.128749 alphamethylacyl-CoA racemase/ |
| | FL = gb: AF047020.1 gb: AF158378.1 gb: NM_014324.1 |
| 9459 | gb: AF047020.1/DEF = Homo sapiens alpha-methylacyl-CoA racemase mRNA, |
| | complete cds./FEA = mRNA/PROD = alpha-methylacyl-CoA racemase/ |
| | DB_XREF = gi: 4204096/UG = Hs.128749 alpha-methylacyl-CoA racemase/ |
| | FL = gb: AF047020.1 gb: AF158378.1 gb: NM_014324.1 |

This gene has been reported in numerous papers including Luo, et al., Molecular Carcinogenesis, 33(1): 25-35 (January 2002); Luo J, et al., Abstract Cancer Res., 62(8): 2220-6 (Apr. 15, 2002).

Table 21 shows the separation with varying number of features for tumor (G3+4) vs. all other tissues.

TABLE 21

| | feat. num. | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 16 | 32 | 64 | 128 |
| 100 * | 92.28 | 93.33 | 93.83 | 94 | 94.33 | 94.43 | 94.1 | 93.8 | 93.43 | 93.53 | 93.45 | 93.37 | 93.18 | 93.03 |
| AUC | | | | | | | | | | | | | | |
| 100 * | 11.73 | 10.45 | 10 | 9.65 | 9.63 | 9.61 | 10.3 | 10.54 | 10.71 | 10.61 | 10.75 | 10.44 | 11.49 | 11.93 |
| AUCstd | | | | | | | | | | | | | | |
| BER (%) | 14.05 | 13.1 | 12.6 | 10.25 | 9.62 | 9.72 | 9.75 | 9.5 | 9.05 | 9.05 | 9.7 | 9.6 | 10.12 | 9.65 |
| BERstd (%) | 13.51 | 12.39 | 12.17 | 11.77 | 9.95 | 10.06 | 10.15 | 10.04 | 9.85 | 10.01 | 10.2 | 10.3 | 10.59 | 10.26 |

Using the same experimental setup, separations were attempted for G4 from non G4, G3 from non G3, Dysplasia from non-dys and Normal from non-Normal. These separations were less successful than the above-described tests, indicating that G3, dysplasia and normal do not have molecular characteristics that distinguish them easily from all other samples. Lists of genes are provided in Tables 22-38. The results suggest making hierarchical decisions as shown in FIG. 6.

Table 22 lists the top 10 genes separating Grade 4 prostate cancer (G4) from all others.

TABLE 22

| | | | Under | | | | |
|---|---|---|---|---|---|---|---|
| | Gene | Unigene | Expr. | | | | Ave. |
| Rank | ID | ID | In G4 | AUC | Pval | FDR | rank |
| 1 | 5923 | Hs.171731 | 1 | 0.9204 | 0.02 | 0.025 | 3.25 |
| 2 | 18122 | Hs.106747 | 1 | 0.9136 | 0.02 | 0.012 | 6.17 |
| 3 | 19573 | Hs.232165 | 1 | 0.9117 | 0.02 | 0.0083 | 7.92 |
| 4 | 893 | Hs.226795 | 1 | 0.9099 | 0.02 | 0.0062 | 7.22 |
| 5 | 9889 | Hs.137569 | 1 | 0.9093 | 0.02 | 0.005 | 8.8 |
| 6 | 19455 | Hs.26892 | 1 | 0.908 | 0.02 | 0.0042 | 10.54 |
| 7 | 19589 | Hs.45140 | 1 | 0.9074 | 0.02 | 0.0036 | 10.54 |
| 8 | 18598 | Hs.9728 | 1 | 0.9062 | 0.02 | 0.0031 | 10.83 |
| 9 | 6519 | Hs.243960 | 1 | 0.9037 | 0.02 | 0.0028 | 12.79 |
| 10 | 11175 | Hs.137569 | 1 | 0.9031 | 0.02 | 0.0025 | 13.46 |

Table 23 below provides the details for the top two genes of this group.

TABLE 23

| Gene ID | Description |
|---|---|
| 5923 | gb: NM_015865.1/DEF = Homo sapiens solute carrier family 14 (urea transporter), |
| | member 1 (Kidd blood group) (SLC14A1), mRNA./FEA = mRNA/ |
| | GEN = SLC14A1/PROD = RACH1/DB_XREF = gi: 7706676/UG = Hs.171731 |
| | solute carrier family 14 (urea transporter), member 1 (Kidd blood group)/ |
| | FL = gb: U35735.1 gb: NM_015865.1 |
| 18122 | gb: NM_021626.1/DEF = Homo sapiens serine carboxypeptidase 1 precursor |
| | protein (HSCP1), mRNA./FEA = mRNA/GEN = HSCP1/PROD = serine |
| | carboxypeptidase 1 precursor protein/DB_XREF = gi: 11055991/UG = Hs.106747 |
| | serine carboxypeptidase 1 precursor protein/FL = gb: AF282618.1 |
| | gb: NM_021626.1 gb: AF113214.1 gb: AF265441.1 |

The following provide the gene descriptions for the top two genes identified in each separation:

Table 24 lists the top 10 genes separating Normal prostate versus all others.

TABLE 24

| | | | Under | | | | |
|---|---|---|---|---|---|---|---|
| | Gene | Unigene | Expr. in | | | | Ave. |
| Rank | ID | ID | Normal | AUC | Pval | FDR | Rank |
| 1 | 6519 | Hs.243960 | −1 | 0.886 | 0.02 | 0.025 | 1.3 |
| 2 | 3448 | Hs.169401 | 1 | 0.8629 | 0.02 | 0.012 | 4.93 |
| 3 | 17900 | Hs.8185 | −1 | 0.8601 | 0.02 | 0.0083 | 6.17 |
| 4 | 6666 | Hs.90911 | −1 | 0.8552 | 0.02 | 0.0062 | 6.59 |
| 5 | 893 | Hs.226795 | −1 | 0.8545 | 0.02 | 0.005 | 7.22 |
| 6 | 6837 | Hs.159330 | −1 | 0.8545 | 0.02 | 0.0042 | 8.05 |
| 7 | 374 | Hs.234642 | −1 | 0.8483 | 0.02 | 0.0036 | 9.69 |
| 8 | 9976 | Hs.103665 | −1 | 0.8458 | 0.02 | 0.0031 | 11.62 |
| 9 | 3520 | Hs.2794 | −1 | 0.8399 | 0.02 | 0.0028 | 15.29 |
| 10 | 3638 | Hs.74120 | −1 | 0.8357 | 0.02 | 0.0025 | 18.17 |

The top two genes are described in detail in Table 25.

TABLE 25

| Gene ID | Description |
|---|---|
| 6519 | gb: NM_016250.1/DEF = Homo sapiens N-myc downstream-regulated gene 2 |
| | (NDRG2), mRNA./FEA = mRNA/GEN = NDRG2/PROD = KIAA1248 protein/ |
| | DB_XREF = gi: 10280619/UG = Hs.243960 N-myc downstream-regulated gene 2/ |
| | FL = gb: NM_016250.1 gb: AF159092. |
| 3448 | gb: N33009/FEA = EST/DB_XREF = gi: 1153408/DB_XREF = est: yy31f09.s1/ |
| | CLONE = IMAGE: 272873/UG = Hs.169401 apolipoprotein E/ |
| | FL = gb: BC003557.1 gb: M12529.1 gb: K00396.1 gb: NM_000041.1 |

Table 26 lists the top 10 genes separating G3 prostate cancer from all others.

TABLE 26

| | | | Under | | | | |
|---|---|---|---|---|---|---|---|
| | Gene | Unigene | Expr. | | | | Ave. |
| Rank | ID | ID | in G3 | AUC | Pval | FDR | rank |
| 1 | 18446 | Hs.283683 | −1 | 0.8481 | 1 | 1.5 | 2.14 |
| 2 | 2778 | Hs.230 | −1 | 0.8313 | 1 | 1.8 | 8.14 |
| 3 | 16102 | Hs.326526 | 1 | 0.8212 | 1 | 2.2 | 10.71 |
| 4 | 12046 | Hs.166982 | 1 | 0.817 | 1 | 2.1 | 15.14 |
| 5 | 9156 | Hs.3416 | −1 | 0.8158 | 1 | 1.8 | 14.71 |
| 6 | 9459 | Hs.128749 | −1 | 0.8158 | 1 | 1.5 | 20.43 |
| 7 | 21442 | Hs.71819 | −1 | 0.8158 | 1 | 1.3 | 13.86 |
| 8 | 6994 | Hs.180248 | −1 | 0.814 | 1 | 1.3 | 11.71 |
| 9 | 17019 | Hs.128749 | −1 | 0.8116 | 1 | 1.3 | 23.14 |
| 10 | 9457 | Hs.128749 | −1 | 0.8074 | 1 | 1.3 | 34.71 |

The top two genes in this group are described in detail in Table 27.

TABLE 27

| Gene ID | Description |
|---|---|
| 18446 | gb: NM_020130.1/DEF = Homo sapiens chromosome 8 open reading frame 4 |
| | (C8ORF4), mRNA./FEA = mRNA/GEN = C8ORF4/PROD = chromosome 8 open |
| | reading frame 4/DB_XREF = gi: 9910147/UG = Hs.283683 chromosome 8 open |
| | reading frame 4/FL = gb: AF268037.1 gb: NM_020130.1 |
| 2778 | gb: NM_002023.2/DEF = Homo sapiens fibromodulin (FMOD), mRNA./ |
| | FEA = mRNA/GEN = FMOD/PROD = fibromodulin precursor/ |
| | DB_XREF = gi: 5016093/UG = Hs.230 fibromodulin/FL = gb: NM_002023.2 |

Table 28 shows the top 10 genes separating Dysplasia from everything else.

TABLE 28

| | | | Under | | | | |
|---|---|---|---|---|---|---|---|
| | Gene | Unigene | Expr. in | | | | Ave. |
| Rank | ID | ID | dysplasia | AUC | Pval | FDR | rank |
| 1 | 5509 | Hs.178121 | −1 | 0.8336 | 0.15 | 0.15 | 4.53 |
| 2 | 4102 | Hs.75426 | −1 | 0.8328 | 0.15 | 0.075 | 4.31 |
| 3 | 10777 | Hs.101047 | 1 | 0.8319 | 0.17 | 0.058 | 5.6 |
| 4 | 18814 | Hs.319088 | 1 | 0.8189 | 0.45 | 0.11 | 10.95 |
| 5 | 4450 | Hs.154879 | 1 | 0.8168 | 0.5 | 0.1 | 11.57 |
| 6 | 14885 | Hs.2554 | 1 | 0.8164 | 0.53 | 0.088 | 18.04 |
| 7 | 10355 | Hs.169832 | 1 | 0.8126 | 0.63 | 0.089 | 14.3 |
| 8 | 5072 | Hs.122647 | −1 | 0.8063 | 0.72 | 0.091 | 26.77 |
| 9 | 3134 | Hs.323469 | −1 | 0.805 | 0.8 | 0.089 | 22.76 |
| 10 | 15345 | Hs.95011 | 1 | 0.8017 | 1 | 0.11 | 29.3 |

Table 29 provides the details for the top two genes listed in Table 28.

TABLE 29

| Gene ID | Description |
|---|---|
| 5509 | gb: NM_021647.1/DEF = Homo sapiens KIAA0626 gene product (KIAA0626), |
| | mRNA./FEA = mRNA/GEN = KIAA0626/PROD = KIAA0626 gene product/ |
| | DB_XREF = gi: 11067364/UG = Hs.178121 KIAA0626 gene product/ |
| | FL = gb: NM_021647.1 gb: AB014526.1 |
| 4102 | gb: NM_003469.2/DEF = Homo sapiens secretogranin II (chromogranin C) |
| | (SCG2), mRNA./FEA = mRNA/GEN = SCG2/PROD = secretogranin II precursor/ |
| | DB_XREF = gi: 10800415/UG = Hs.75426 secretogranin II (chromogranin C)/ |
| | FL = gb: NM_003469.2 gb: M25756.1 |

To support the proposed decision tree of FIG. 6, classifiers are needed to perform the following separations: G3 vs. G4; NL vs. Dys.; and TZG4 vs. PZG4.

Due to the small sample sizes, poor performance was obtained with 10×10-fold cross-validation. To avoid this problem, leave-one-out cross-validation was used instead. In doing so, the average AUC for all repeats cannot be reported because there is only one test example in each repeat. Instead, the leave-one-out error rate and the pooled AUC are evaluated. However, all such pairwise separations are difficult to achieve with high accuracy and a few features.

Table 30 lists the top 10 genes separating G3 from G4. Table 31 provides the details for the top two genes listed.

TABLE 30

| | | | (+) Expr. | | | | |
|---|---|---|---|---|---|---|---|
| | | | in G4; | | | | |
| | Gene | Unigene | (−) Expr. | | | | Ave. |
| Rank | ID | ID | in G3 | AUC | Pval | FDR | rank |
| 1 | 19455 | Hs.26892 | −1 | 0.9057 | 0.45 | 0.45 | 1.09 |
| 2 | 11175 | Hs.137569 | −1 | 0.8687 | 1 | 1.8 | 2.95 |
| 3 | 9156 | Hs.3416 | −1 | 0.8653 | 1 | 1.4 | 4 |
| 4 | 18904 | Hs.315167 | 1 | 0.8653 | 1 | 1.1 | 4.71 |
| 5 | 9671 | Hs.98658 | 1 | 0.8636 | 1 | 0.99 | 5.45 |
| 6 | 2338 | Hs.62661 | −1 | 0.8586 | 1 | 0.96 | 6.64 |
| 7 | 2939 | Hs.82906 | 1 | 0.8586 | 1 | 0.82 | 7.46 |
| 8 | 450 | Hs.27262 | 1 | 0.8552 | 1 | 0.8 | 8.44 |
| 9 | 18567 | Hs.193602 | 1 | 0.8535 | 1 | 0.85 | 9.49 |
| 10 | 5304 | Hs.252136 | −1 | 0.8519 | 1 | 0.77 | 10.67 |

TABLE 31

| Gene ID | Description |
|---|---|
| 19455 | gb: NM_018456.1/DEF = Homo sapiens uncharacterized |
| | bone marrow protein BM040 (BM040), mRNA./FEA = |
| | mRNA/GEN = BM040/PROD = uncharacterized bone |
| | marrow protein BM040/DB_XREF = gi: 8922098/ |
| | UG = Hs.26892 uncharacterized bone marrow protein |
| | BM040/FL = gb: AF217516.1 gb: NM_018456.1 |
| 11175 | gb: AB010153.1/DEF = Homo sapiens mRNA for p73H, |
| | complete cds./FEA = mRNA/GEN = p73H/PROD = p73H/ |
| | DB_XREF = gi: 3445483/UG = Hs.137569 tumor protein 63 |
| | kDa with strong homology to p53/FL = gb: AB010153.1 |

Table 32 lists the top 10 genes for separating Normal prostate from Dysplasia. Details of the top two genes for performing this separation are provided in Table 33.

TABLE 32

| | | | (−) Expr. | | | | |
|---|---|---|---|---|---|---|---|
| | | | in NL; | | | | |
| | Gene | Unigene | (+) Expr. | | | | Ave. |
| Rank | ID | ID | in Dys | AUC | Pval | FDR | rank |
| 1 | 4450 | Hs.154879 | −1 | 0.9037 | 0.05 | 0.05 | 1.09 |
| 2 | 10611 | Hs.41682 | 1 | 0.8957 | 0.075 | 0.037 | 2.02 |
| 3 | 9048 | Hs.177556 | −1 | 0.8743 | 0.45 | 0.15 | 3.17 |
| 4 | 18069 | Hs.103147 | −1 | 0.8717 | 0.57 | 0.14 | 4.06 |
| 5 | 7978 | Hs.20815 | −1 | 0.8583 | 1 | 0.23 | 5.56 |
| 6 | 6837 | Hs.159330 | −1 | 0.8556 | 1 | 0.21 | 6.37 |
| 7 | 7229 | Hs.71816 | −1 | 0.8463 | 1 | 0.34 | 8.03 |
| 8 | 21059 | Hs.283753 | 1 | 0.8449 | 1 | 0.3 | 9.51 |
| 9 | 15345 | Hs.95011 | −1 | 0.8436 | 1 | 0.29 | 9.94 |
| 10 | 2463 | Hs.91251 | −1 | 0.8369 | 1 | 0.38 | 11.78 |

TABLE 33

| Gene ID | Description |
|---|---|
| 4450 | gb: NM_022719.1/DEF = Homo sapiens DiGeorge syndrome critical region gene |
| | DGSI (DGSI), mRNA./FEA = mRNA/GEN = DGSI/PROD = DiGeorge syndrome |
| | critical region gene DGSIprotein/DB_XREF = gi: 13027629/UG = Hs.154879 |
| | DiGeorge syndrome critical region gene DGSI/FL = gb: NM_022719.1 |
| 10611 | gb: U30610.1/DEF = Human CD94 protein mRNA, complete cds./FEA = mRNA/ |
| | PROD = CD94 protein/DB_XREF = gi: 1098616/UG = Hs.41682 killer cell lectin- |
| | like receptor subfamily D, member 1/FL = gb: U30610.1 gb: NM_002262.2 |

Table 34 lists the top 10 genes for separating peripheral zone G4 prostate cancer from transition zone G4 cancer. Table 35 provides the details for the top two genes in this separation.

TABLE 34

| | | | (−) Expr. | | | | |
|---|---|---|---|---|---|---|---|
| | | | in TZ; | | | | |
| | Gene | Unigene | (+) Expr. | | | | Ave. |
| Rank | ID | ID | In PZ | AUC | Pval | FDR | rank |
| 1 | 4654 | Hs.194686 | 1 | 0.9444 | 1 | 1.2 | 1.1 |
| 2 | 14953 | Hs.306423 | 1 | 0.9306 | 1 | 1.1 | 2.45 |
| 3 | 929 | Hs.279949 | −1 | 0.9167 | 1 | 1.7 | 4 |
| 4 | 6420 | Hs.274981 | 1 | 0.9167 | 1 | 1.3 | 4.84 |
| 5 | 7226 | Hs.673 | 1 | 0.9167 | 1 | 1 | 5.69 |
| 6 | 18530 | Hs.103291 | 1 | 0.9167 | 1 | 0.86 | 6.68 |
| 7 | 6618 | Hs.2563 | 1 | 0.9097 | 1 | 1.1 | 7.82 |
| 8 | 16852 | Hs.75626 | 1 | 0.9097 | 1 | 0.93 | 8.91 |
| 9 | 19242 | Hs.12692 | 1 | 0.9097 | 1 | 0.82 | 9.78 |
| 10 | 6106 | Hs.56294 | 1 | 0.9063 | 1 | 1 | 10.75 |

TABLE 35

| Gene ID | Description |
|---|---|
| 4654 | gb: NM_003951.2/DEF = Homo sapiens solute carrier family 25 (mitochondrial |
| | carrier, brain), member 14 (SLC25A14), transcript variant long, nuclear gene |
| | encoding mitochondrial protein, mRNA./FEA = mRNA/GEN = SLC25A14/ |
| | PROD = solute carrier family 25, member 14, isoformUCP5L/ |
| | DB_XREF = gi: 6006039/UG = Hs.194686 solute carrier family 25 (mitochondrial |
| | carrier, brain), member 14/FL = gb: AF155809.1 gb: AF155811.1 |
| | gb: NM_022810.1 gb: AF078544.1 gb: NM_003951.2 |
| 14953 | gb: AK002179.1/DEF = Homo sapiens cDNA FLJ11317 fis, clone |
| | PLACE1010261, moderately similar to SEGREGATION DISTORTER |
| | PROTEIN./FEA = mRNA/DB_XREF = gi: 7023899/UG = Hs.306423 Homo |
| | sapiens cDNA FLJ11317 fis, clone PLACE1010261, moderately similar to |
| | SEGREGATION DISTORTER PROTEIN |

As stated in an earlier discussion, PSA is not predictive of tissue malignancy. There is very little correlation of PSA and cancer volume (R2=0.316). The R2 was also computed for PSA vs. prostate weight (0.025) and PSA vs. CA/Weight (0.323). PSA does not separate well the samples in malignancy categories. In this data, there did not appear to be any correlation between PSA and prostate weight.

A test was conducted to identify the genes most correlated with PSA, in BPH samples or in G3/4 samples, which were found to be genes 11541 for BPH and 14523 for G3/4. The details for these genes are listed below in Table 36.

TABLE 36

| Gene ID | Description |
|---|---|
| 11541 | gb: AB050468.1/DEF = Homo sapiens mRNA for membrane glycoprotein LIG-1, |
| | complete cds./FEA = mRNA/GEN = lig-1/PROD = membrane glycoprotein LIG-1/ |
| | DB_XREF = gi: 13537354/FL = gb: AB050468.1 |
| 14523 | gb: AL046992/FEA = EST/DB_XREF = gi: 5435048/ |
| | DB_XREF = est: DKFZp586L0417_r1/CLONE = DKFZp586L0417/ |
| | UG = Hs.184907 G protein-coupled receptor 1/FL = gb: NM_005279.1 |
| 5626 | gb: NM_006200.1/DEF = Homo sapiens proprotein convertase subtilisinkexin |
| | type 5 (PCSK5), mRNA./FEA = mRNA/GEN = PCSK5/PROD = proprotein |
| | convertase subtilisinkexin type 5/DB_XREF = gi: 11321618/UG = Hs.94376 |
| | proprotein convertase subtilisinkexin type 5/FL = gb: NM_006200.1 gb: U56387.2 |

Gene 11541 shows no correlation with PSA in G3/4 samples, whereas gene 14523 shows correlation in BPH samples. Thus, 11541 is possibly the result of some overfitting due to the fact that pre-operative PSAs are available for only 7 BPH samples. Gene 14523 appears to be the most correlated gene with PSA in all samples. Gene 5626, also listed in Table 34, has good correlation coefficients (R_{BPH}²=0.44, R_{G34}²=0.58).

Reports are found in the published literature indicating that G Protein-coupled receptors such as gene 14523 are important in characterizing prostate cancer. See, e.g. L. L. Xu, et al. Cancer Research 60, 6568-6572, Dec. 1, 2000.

For comparison, genes that have “prostate specific antigen” in their description (none had PSA) were considered:

Gene 4649: gb:NM_{—}001648.1/DEF=Homo sapiens kallikrein 3, (prostate specific antigen) (KLK3), mRNA. /FEA=mRNA /GEN=KLK3/PROD=kallikrein 3, (prostate specific antigen)/DB_XREF=gi:4502172/UG=Hs.171995 kallikrein 3, (prostate specific antigen) /FL=gb:BC005307.1 gb:NM_{—}001648.1 gb:U17040.1 gb:M26663.1; and gene 4650: gb:U17040.1/DEF=Human prostate specific antigen precursor mRNA, complete cds. /FEA=mRNA /PROD=prostate specific antigen precursor /DB_XREF=gi:595945/UG=Hs.171995 kallikrein 3, (prostate specific antigen) /FL=gb:BC005307.1 gb:NM_{—}001648.1 gb:U17040.1 gb:M26663.1. Neither of these genes had activity that correlates with preoperative PSA.

Another test looked at finding genes whose expression correlate with cancer volume in grade 3 and 4 cancer tissues. However, even the most correlated gene is not found significant with respect to the Bonferroni-corrected pvalue (pval=0.42). Table 37 lists the top nine genes most correlated with cancer volume in G3+4 samples. The details of the top gene are provided in Table 38.

TABLE 37

| | Gene | Unigene | Sign | | | |
|---|---|---|---|---|---|---|
| Rank | ID | ID | corr. | Pearson | Pval | FDR |
| 1 | 8851 | Hs.217493 | −1 | 0.6582 | 0.43 | 0.43 |
| 2 | 6892 | Hs.2868 | −1 | 0.6282 | 1 | 0.51 |
| 3 | 21353 | Hs.283803 | 1 | 0.6266 | 1 | 0.36 |
| 4 | 7731 | Hs.182507 | −1 | 0.6073 | 1 | 0.53 |
| 5 | 4853 | Hs.86958 | −1 | 0.6039 | 1 | 0.46 |
| 6 | 622 | Hs.14449 | −1 | 0.5958 | 1 | 0.48 |
| 7 | 8665 | Hs.74497 | 1 | 0.5955 | 1 | 0.41 |
| 8 | 13750 | Hs.2014 | −1 | 0.579 | 1 | 0.6 |
| 9 | 15413 | Hs.177961 | −1 | 0.5775 | 1 | 0.56 |

TABLE 38

| Gene ID | Description |
|---|---|
| 8851 | gb: M62898.1/DEF = Human lipocortin (LIP) 2 pseudogene |
| | mRNA, complete cdslike region./FEA = mRNA/DB_XREF = |
| | gi: 187147/UG = Hs.217493 annexin A2/FL = gb: M62898.1 |

A lipocortin has been described in U.S. Pat. No. 6,395,715 entitled “Uteroglobin gene therapy for epithelial cell cancer”. Using RT-PCR, under-expression of lipocortin in cancer compared to BPH has been reported by Kang J S et al., Clin Cancer Res. 2002 January; 8(1):117-23.

### Example 6

### Prostate Cancer Comparative Study of Stamey Data (12-2004)

In this example sets of genes obtained with two different data sets are compared. Both data sets were generated by Dr. Stamey of Stanford University, the first in 2001 using Affymetrix HuGeneFL probe arrays, the second in 2003 using Affymetrix U133A chip. After matching the genes in both arrays, a set of about 2000 common genes. Gene selection was performed on the data of both studies independently, then the gene sets obtained were compared. A remarkable agreement is found. In addition, classifiers were trained on one dataset and tested on the other. In the separation tumor (G3/4) vs. all other tissues, classification accuracies comparable to those obtained in previous reports were obtained by cross-validation on the second study: 10% error can be achieved with 10 genes (on the independent test set of the first study); by cross-validation, there was 8% error. In the separation BPH vs. all other tissues, there was also 10% error with 10 genes. The cross-validation results for BPH were overly optimistic (only one error), however this was not unexpected since there were only 10 BPH samples in the second study. Tables of genes were selected by consensus of both studies.

The 2001 (first) data set consists of 67 samples from 26 patients. The Affymetrix HuGeneFL probe arrays used have 7129 probes, representing ˜6500 genes. The composition of the 2001 dataset (number of samples in parenthesis) is summarized in Table 39. Several grades and zones are represented, however, all TZ samples are BPH (no cancer), all CZ samples are normal (no cancer). Only the PZ contains a variety of samples. Also, many samples came from the same tissues.

TABLE 39

| Zone | Histological classification |
|---|---|
| CZ(3) | NL(3) |
| PZ (46) | NL (5) |
| | Stroma(1) |
| | Dysplasia (3) |
| | G3 (10) |
| | G4 (27) |
| TZ(18) | BPH(18) |
| Total 67 | |

The 2003 (second) dataset consists of a matrix of 87 lines (samples) and 22283 columns (genes) obtained from an Affymetrix U133A chip. The distribution of the samples of the microarray prostate cancer study is given as been provided previously in Table 12.

Genes that had the same Gene Accession Number (GAN) in the two arrays HuGeneFL and U133A were selected. The selection was further limited to descriptions that matched reasonably well. For that purpose, a list of common words was created. A good match corresponds to a pair of description having at least a common word, excluding these common words, short word (less that 3 letters) and numbers. The results was a set of 2346 genes.

Because the data from both studies came normalized in different ways, it was re-normalized using the routine provided below. Essentially, the data is translated and scaled, the log is taken, the lines and columns are normalized, the outlier values are squashed. This preprocessing was selected based on a visual examination of the data.

For the 2001 study, a bias of −0.08 was used. For the 2003 study, the bias was 0. Visual examination revealed that these values stabilize the variance of both classes reasonably well.

The set of 2346 genes was ranked using the data of both studies independently, with the area under the ROC curve (AUC) being used as the ranking criterion. P values were computed with the Bonferroni correction and False discovery rate (FDR) was calculated.

Both rankings were compared by examining the correlation of the AUC scores. Cross-comparisons were done by selecting the top 50 genes in one study and examining how “enriched” in those genes were the lists of top ranking genes from the other study, varying the number of genes. This can be compared to a random ranking. For a consensus ranking, the genes were ranked according to their smallest score in the two studies.

Reciprocal tests were run in which the data from one study was used for training of the classifier which was then tested on the data from the other study. Three different classifiers were used: Linear SVM, linear ridge regression, and Golub's classifier (analogous to Naïve Bayes). For every test, the features selected with the training set were used. For comparison, the consensus features were also used.

Separation of all tumor samples (G3 and G4) from all others was performed, with the G3 and G4 samples being grouped into the positive class and all samples grouped into the negative class. The genes were ranked in two ways, using the data of the first study (2001) and using the data of the second study (2003)

Most genes ranking high in one study also rank high in the other, with some notable exceptions. These exceptions may correspond to probes that do not match in both arrays even though their gene identification and descriptions match. They may also correspond to probes that “failed” to work in one array.

Table 40 lists the top 25 genes resulting from the feature ranking by consensus between the 2001 study and the 2003 study Tumor G3/4 vs. others. Ranking is performed according to a score that is the minimum of score0 and score1.

TABLE 40

| | Unigene | Over | | | | | | |
|---|---|---|---|---|---|---|---|---|
| Rk | ID | Expr | Scor | Rk0 | Score0 | Rk1 | Score1 | Description |
| 1 | Hs.195850 | −1 | 0.8811 | 7 | 0.8811 | 2 | 0.8813 | Human keratin type II (58 kD) |
| | | | | | | | | mRNA |
| 2 | Hs.171731 | −1 | 0.8754 | 1 | 0.9495 | 3 | 0.8754 | Human RACH1 (RACH1) mRNA |
| 3 | Hs.65029 | −1 | 0.8647 | 8 | 0.8802 | 5 | 0.8647 | Human gas1 gene |
| 4 | Hs.771 | −1 | 0.8532 | 15 | 0.8532 | 1 | 0.8953 | Human liver glycogen |
| | | | | | | | | phosphorylase mRNA |
| 5 | Hs.79217 | 1 | 0.8532 | 16 | 0.8532 | 7 | 0.855 | Human pyrroline 5-carboxylate |
| | | | | | | | | reductase mRNA |
| 6 | Hs.198760 | −1 | 0.8495 | 19 | 0.8495 | 4 | 0.869 | H. sapiens NF-H gene |
| 7 | Hs.174151 | −1 | 0.8448 | 4 | 0.8892 | 10 | 0.8448 | Human aldehyde oxidase (hAOX) |
| | | | | | | | | mRNA |
| 8 | Hs.44 | −1 | 0.841 | 12 | 0.8685 | 14 | 0.841 | Human nerve growth factor |
| | | | | | | | | (HBNF-1) mRNA |
| 9 | Hs.3128 | 1 | 0.841 | 2 | 0.9081 | 15 | 0.841 | Human RNA polymerase II |
| | | | | | | | | subunit (hsRPB8) mRNA |
| 10 | Hs.34853 | −1 | 0.8314 | 5 | 0.8892 | 20 | 0.8314 | Human Id-related helix-loop- |
| | | | | | | | | helix protein Id4 mRNA |
| 11 | Hs.113 | −1 | 0.8217 | 13 | 0.8658 | 24 | 0.8217 | Human cytosolic epoxide |
| | | | | | | | | hydrolase mRNA |
| 12 | Hs.1813 | −1 | 0.8201 | 31 | 0.827 | 25 | 0.8201 | Homo sapiens synaptic vesicle |
| | | | | | | | | amine transporter (SVAT) mRNA |
| 13 | Hs.2006 | −1 | 0.8099 | 40 | 0.8099 | 23 | 0.8255 | Human glutathione transferase |
| | | | | | | | | M3 (GSTM3) mRNA |
| 14 | Hs.76224 | −1 | 0.8083 | 28 | 0.836 | 39 | 0.8083 | Human extracellular protein |
| | | | | | | | | (S1-5) mRNA |
| 15 | Hs.27311 | 1 | 0.8056 | 11 | 0.8694 | 42 | 0.8056 | Human transcription factor |
| | | | | | | | | SIM2 long form mRNA |
| 16 | Hs.77546 | −1 | 0.8008 | 14 | 0.8649 | 46 | 0.8008 | Human mRNA for KIAA0172 |
| | | | | | | | | gene |
| 17 | Hs.23838 | 1 | 0.7982 | 50 | 0.7982 | 22 | 0.8287 | Human neuronal DHP-sensitive |
| 18 | Hs.10755 | −1 | 0.7955 | 53 | 0.7955 | 17 | 0.8373 | Human mRNA for |
| | | | | | | | | dihydropyrimidinase |
| 19 | Hs.2785 | −1 | 0.7911 | 24 | 0.8414 | 51 | 0.7911 | H. sapiens gene for cytokeratin |
| | | | | | | | | 17 |
| 20 | Hs.86978 | 1 | 0.7748 | 75 | 0.7748 | 70 | 0.7777 | H. sapiens mRNA for prolyl |
| | | | | | | | | oligopeptidase |
| 21 | Hs.2025 | −1 | 0.7744 | 3 | 0.9027 | 73 | 0.7744 | Human transforming growth factor- |
| | | | | | | | | beta 3 (TGF-beta3) mRNA |
| 22 | Hs.30054 | 1 | 0.7734 | 45 | 0.8054 | 74 | 0.7734 | Human coagulation factor V mRNA |
| 23 | Hs.155591 | −1 | 0.7723 | 52 | 0.7973 | 76 | 0.7723 | Human forkhead protein FREAC-1 |
| | | | | | | | | mRNA |
| 24 | Hs.237356 | −1 | 0.7712 | 81 | 0.7712 | 61 | 0.7846 | Human intercrine-alpha (hIRH) |
| | | | | | | | | mRNA |
| 25 | Hs.211933 | −1 | 0.7707 | 70 | 0.7784 | 80 | 0.7707 | Human (clones HT-[125 |

Training of the classifier was done with the data of one study while testing used the data of the other study. The results are similar for the three classifiers that were tried: SVM, linear ridge regression and Golub classifier. Approximately 90% accuracy can be achieved in both cases with about 10 features. Better “cheating” results are obtained with the consensus features. This serves to validate the consensus features, but the performances cannot be used to predict the accuracy of a classifier on new data. An SVM was trained using the two best features of the 2001 study and the sample of the 2001 study as the training data. The samples from the 2003 study were used as test data to achieve an error rate of 16% is achieved. The tumor and non-tumor samples are well separated, but that, in spite of normalization, the distributions of the samples is different between the two studies.

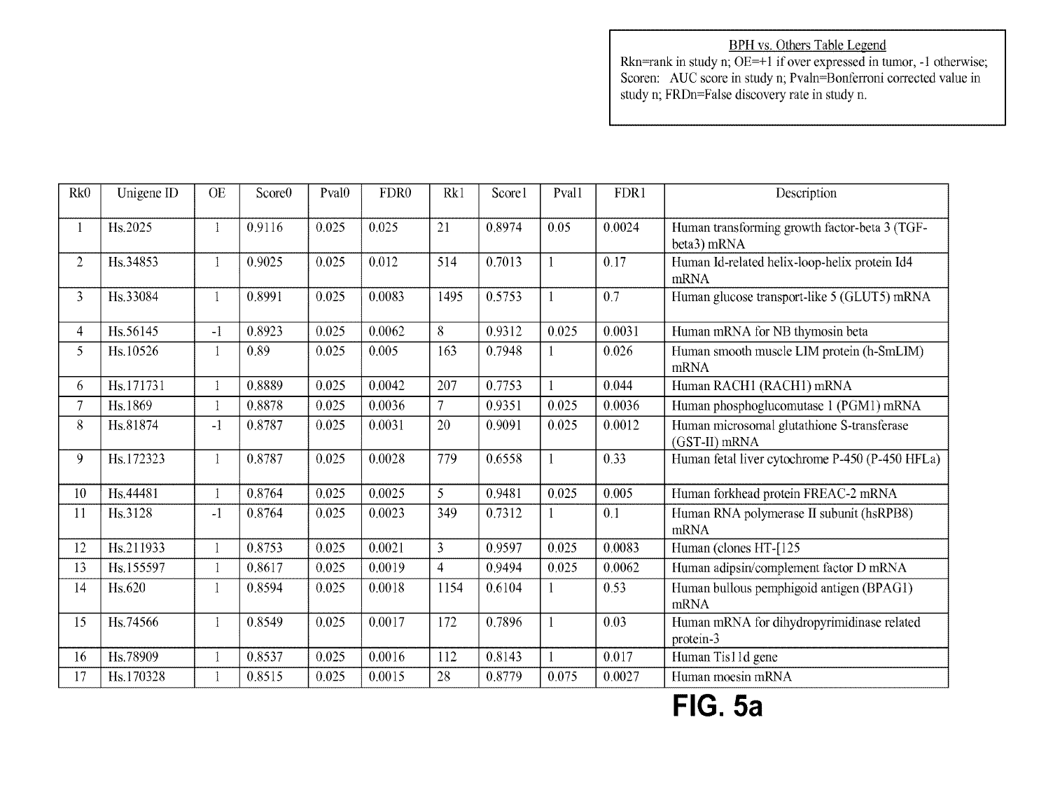

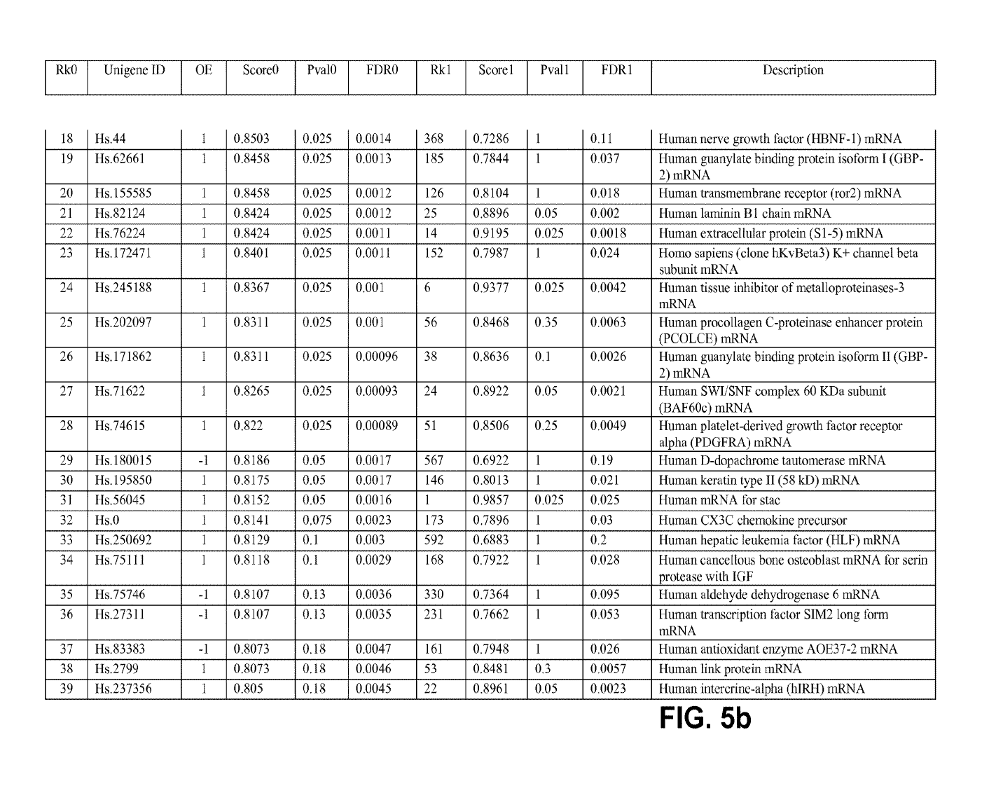

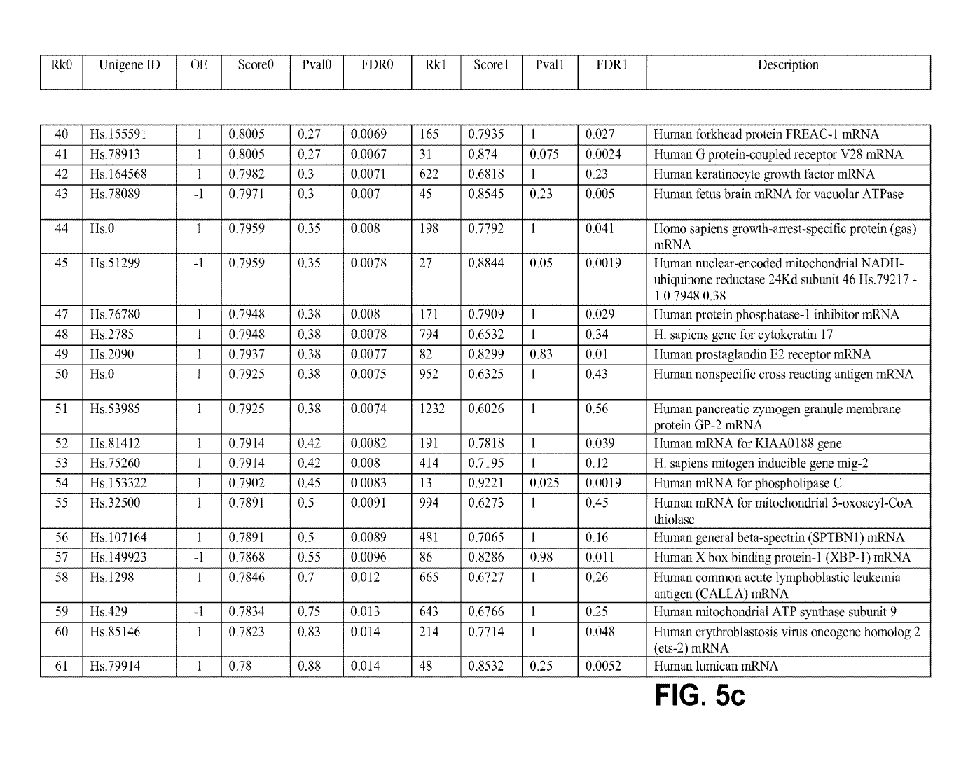

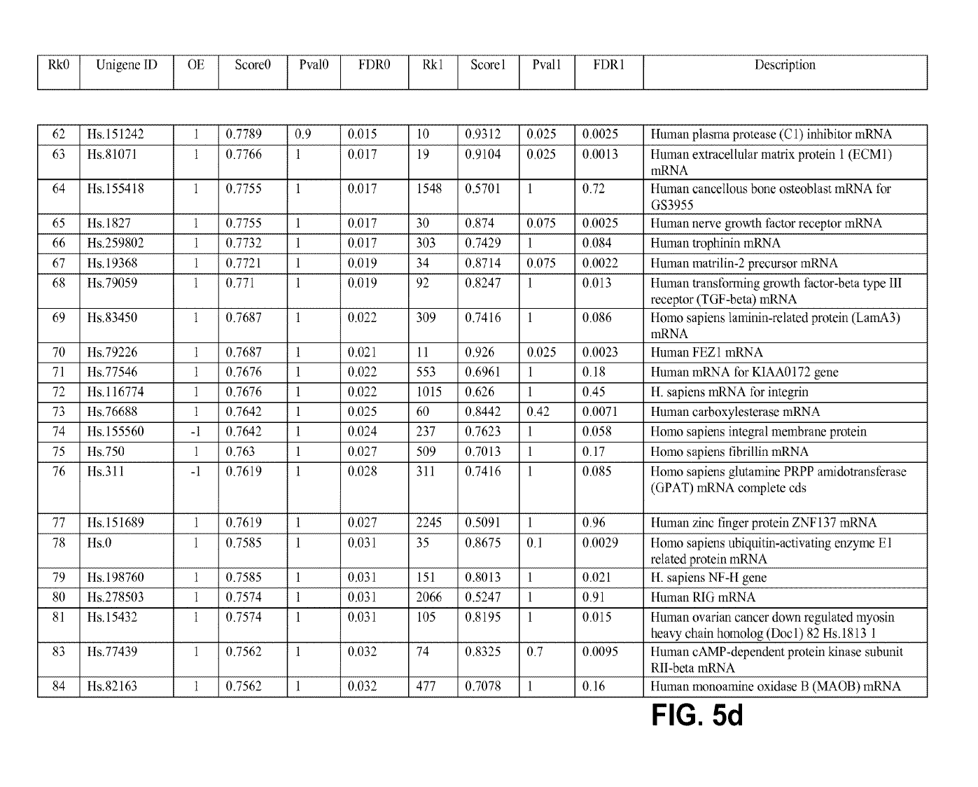

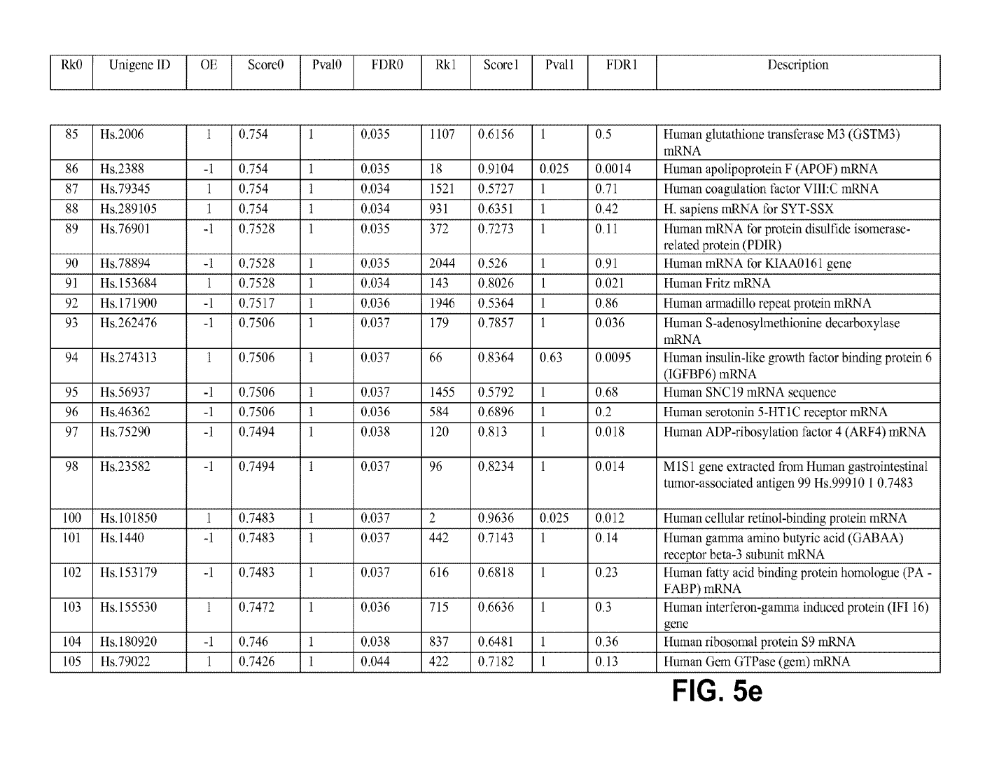

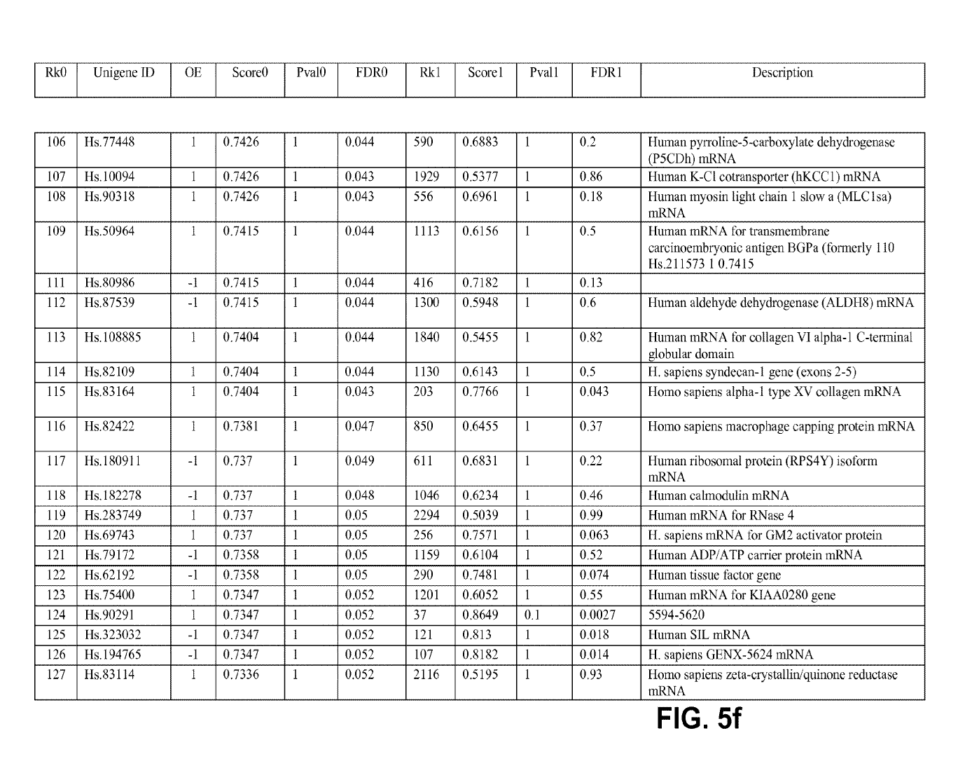

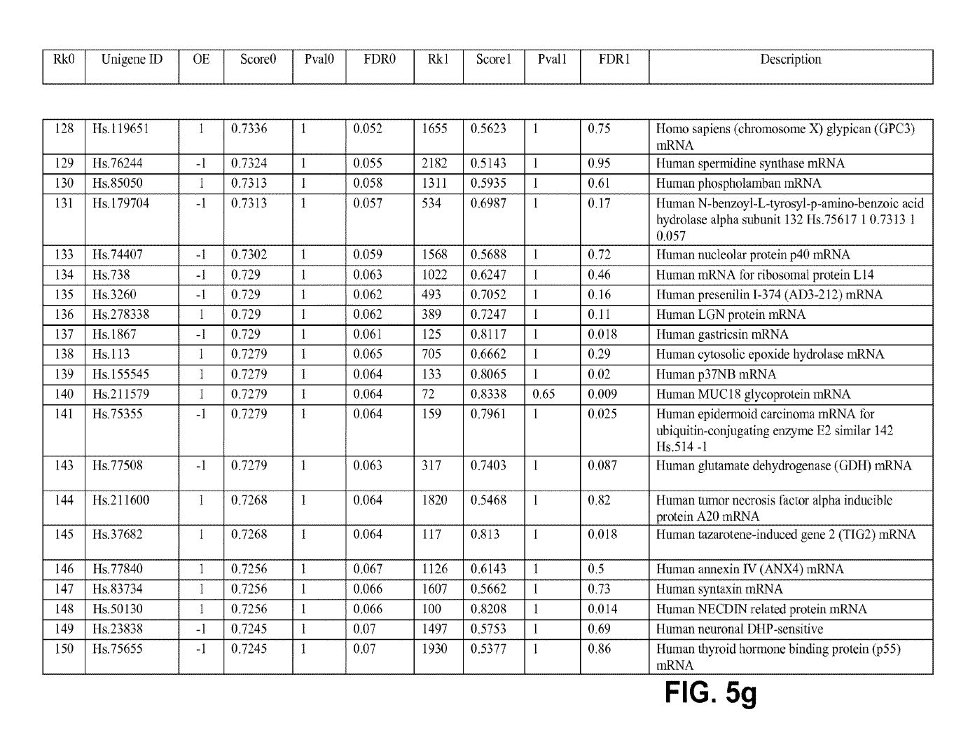

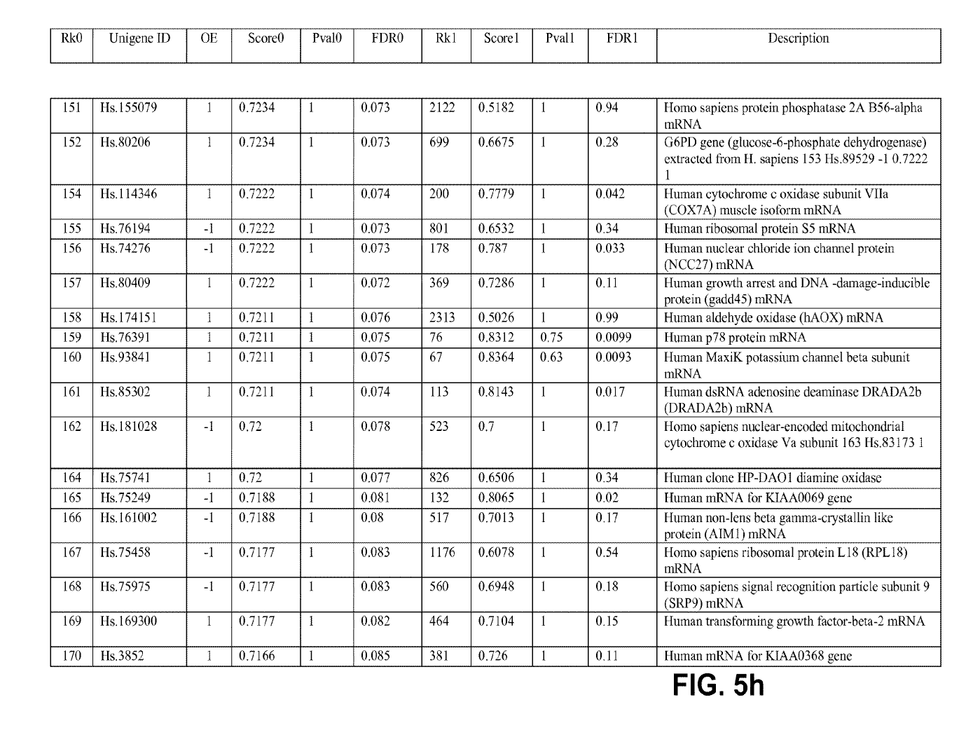

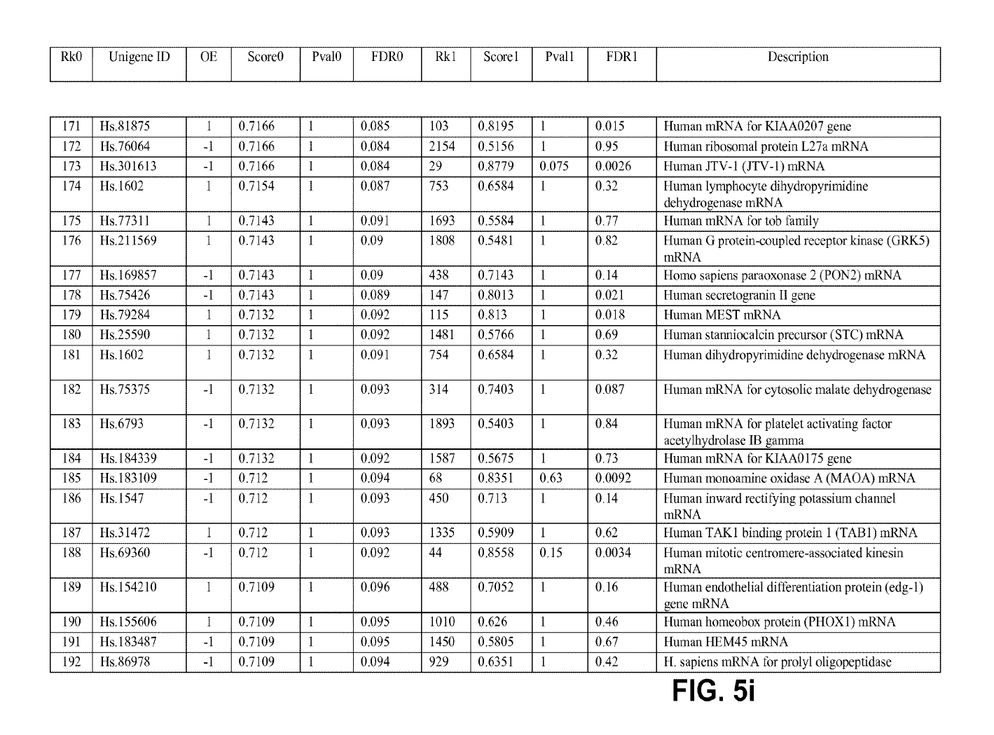

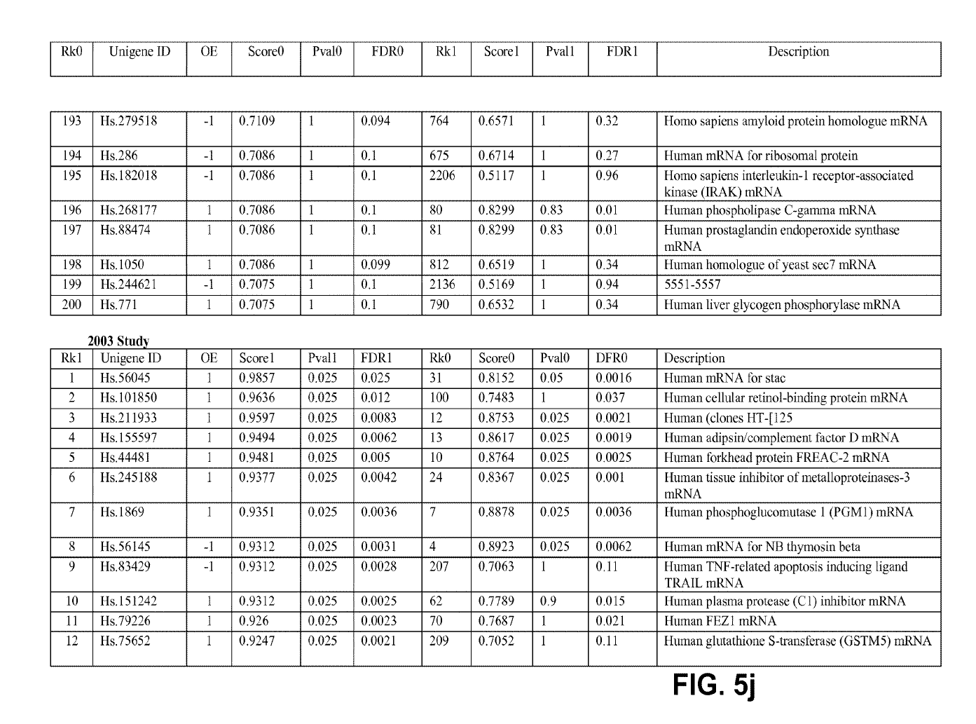

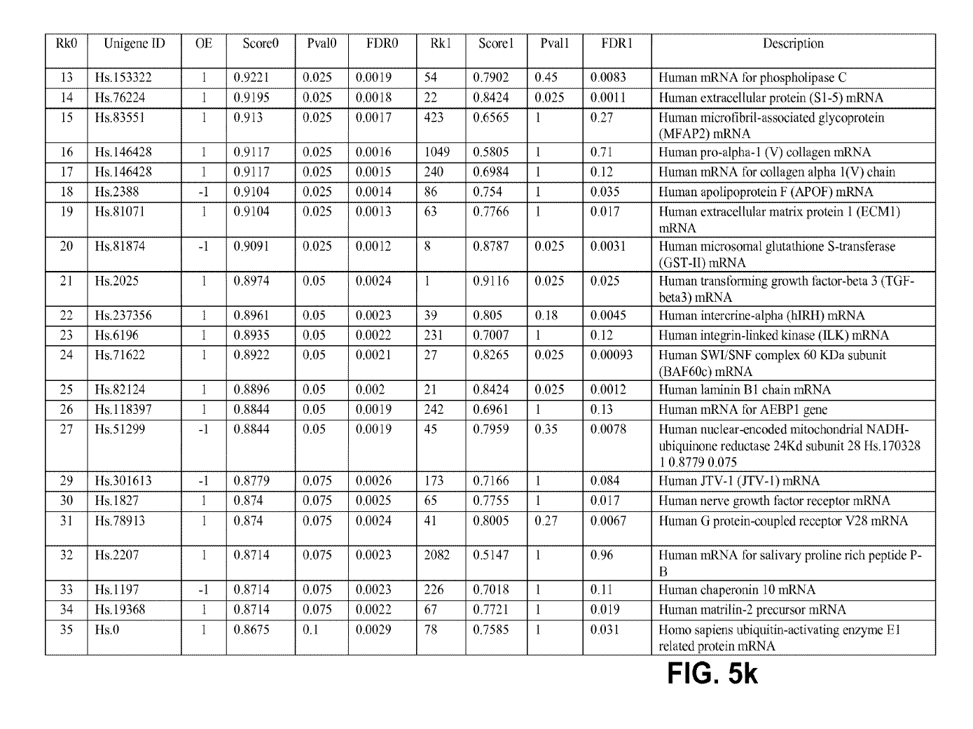

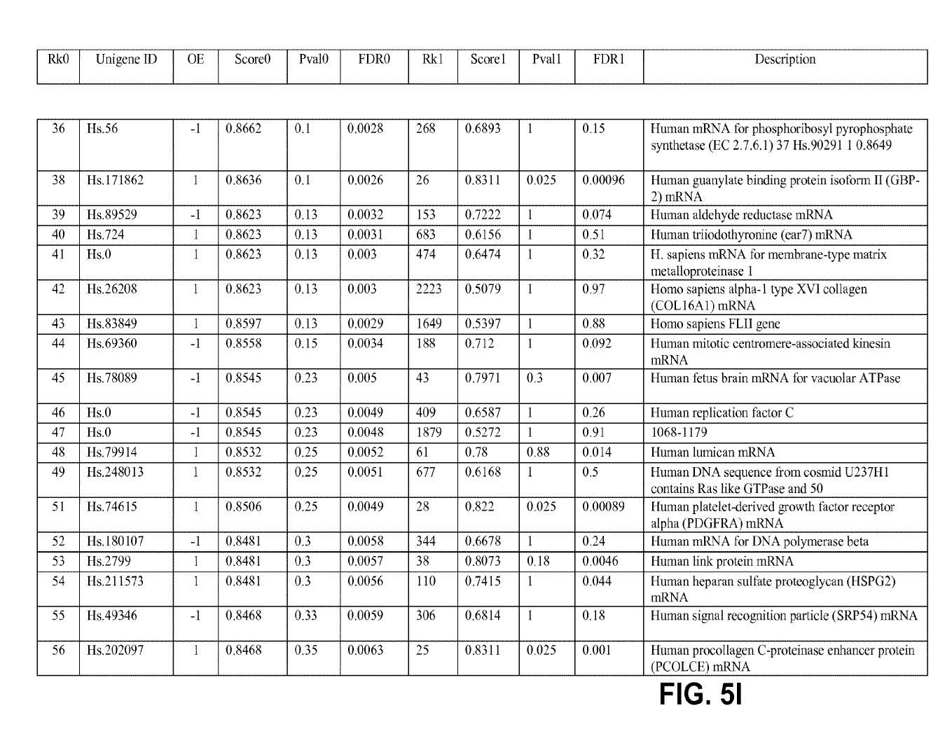

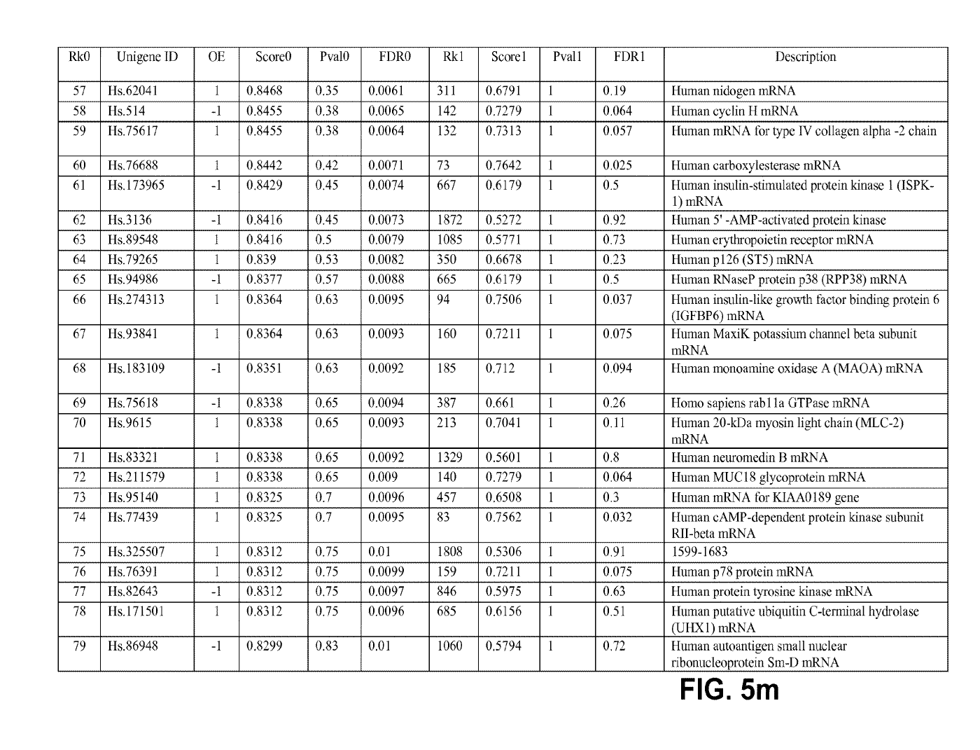

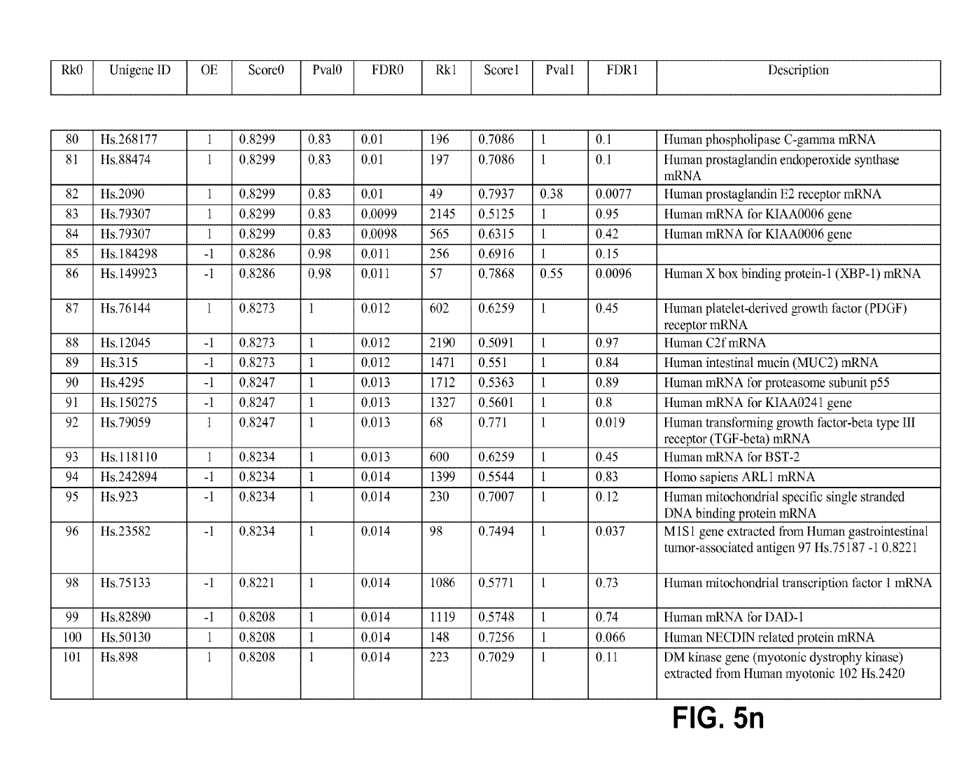

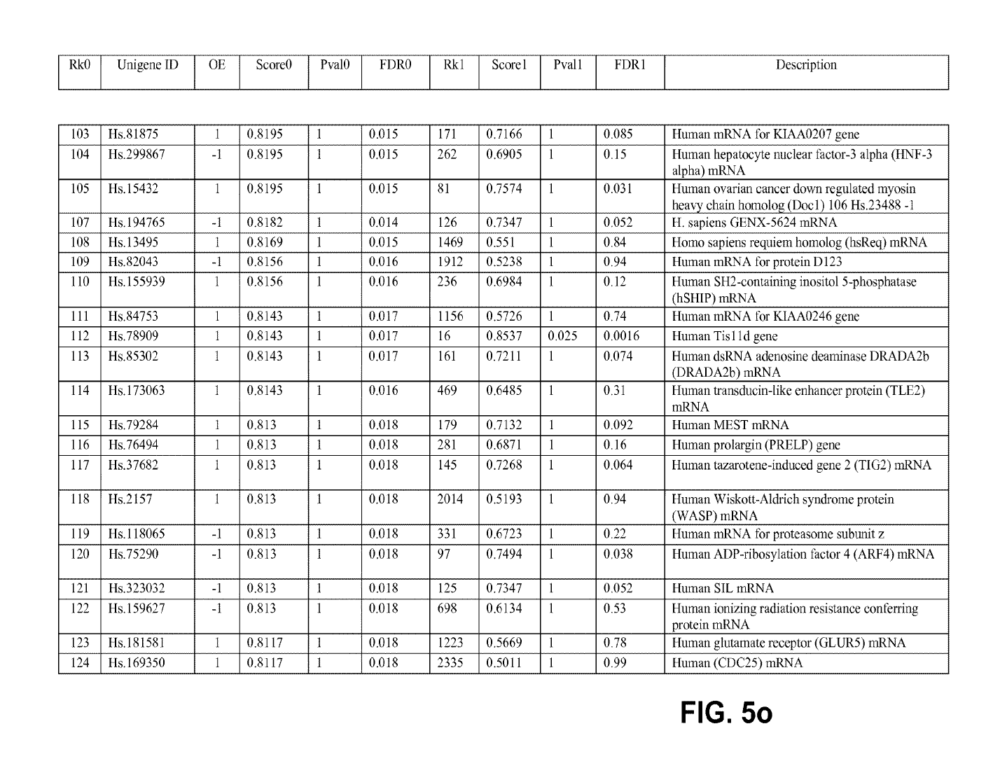

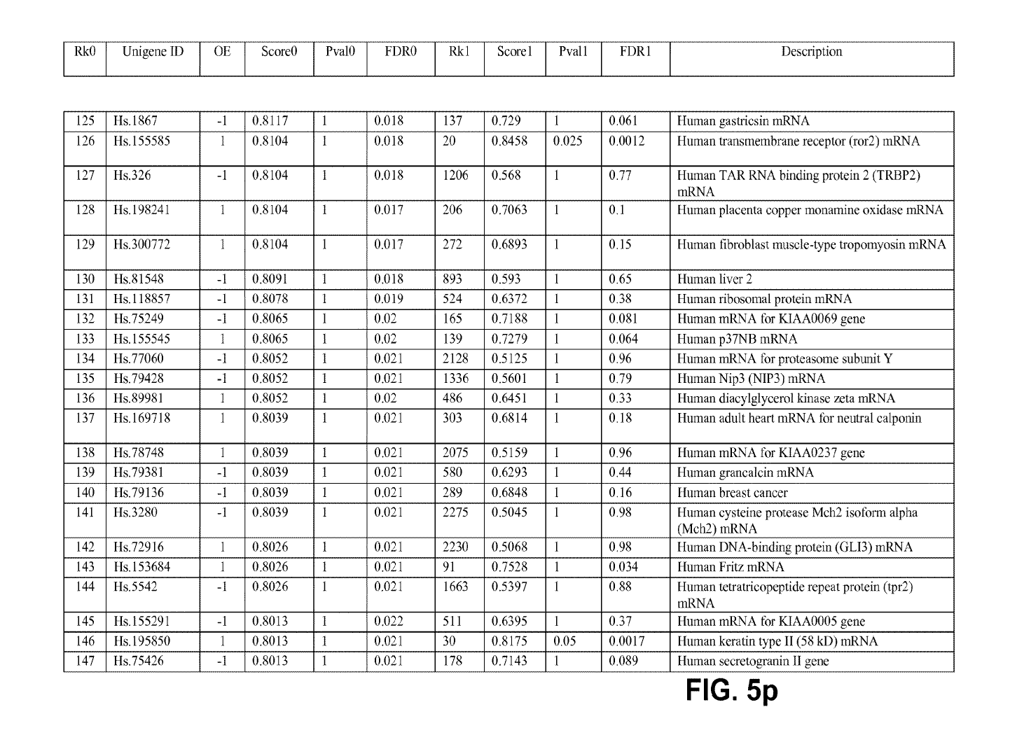

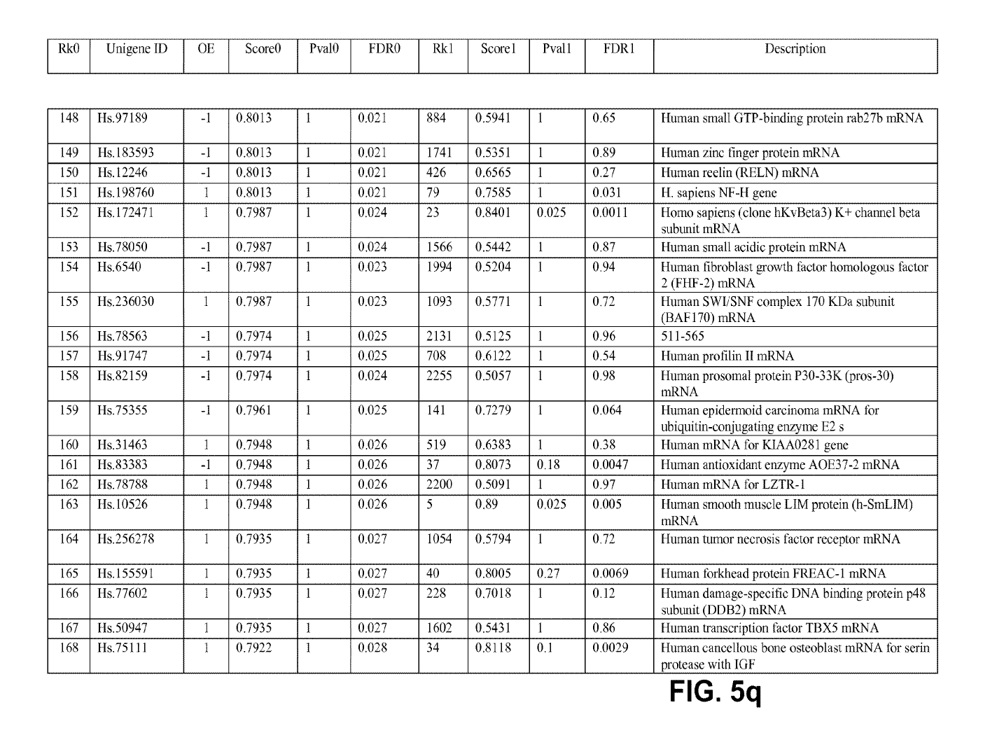

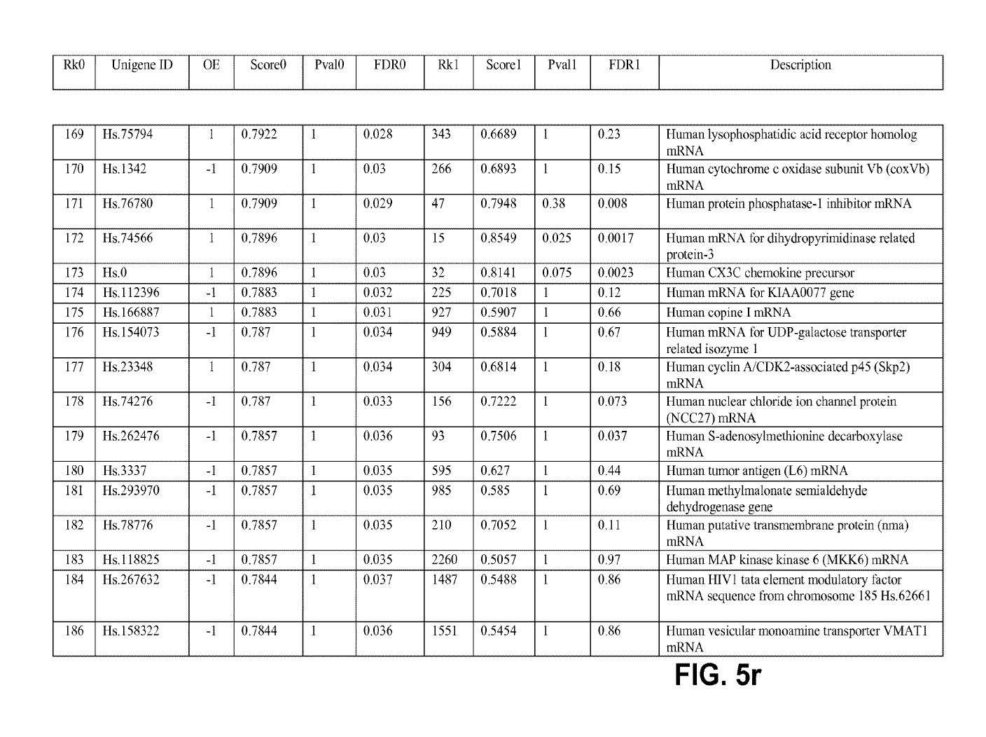

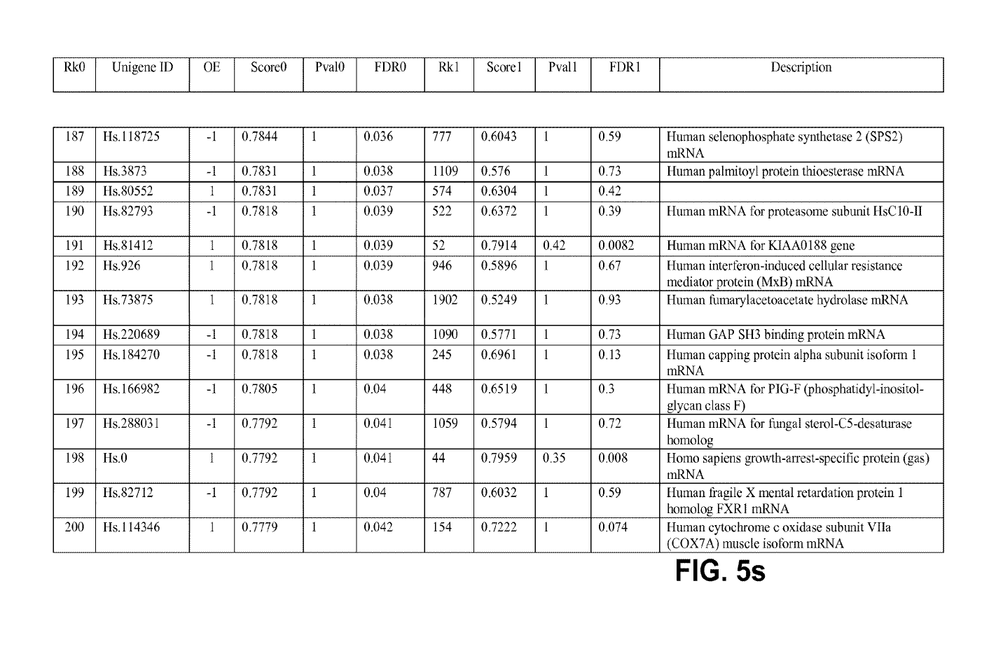

The same procedures as above were repeated for the separation of BPH vs. all other tissues. The correlation between the scores of the genes obtained in both studies was investigated. The Pearson correlation is R=0.37, smaller than the value 0.46 found in the separation tumor vs. others. FIG. 5a-s provides tables of genes ranked by either study for BPH vs. others. The genes are ranked in two ways, using the data of the first study (2001) and using the data of the second study (2003). The genes are ranked according to a score that is the minimum of score0 and score1. Table 41 lists the top 50 for the BPH vs. others feature ranking by consensus between the 2001 study and the 2003 study.

TABLE 41

| | Unigene | | | | | | | |
|---|---|---|---|---|---|---|---|---|
| RK | ID | OE | Score | Rk0 | Score0 | Rk1 | Score1 | Description |
| 1 | Hs.2025 | 1 | 0.8974 | 1 | 0.9116 | 21 | 0.8974 | Human transforming growth factor- |
| | | | | | | | | beta 3 (TGF-beta3) mRNA |
| 2 | Hs.56145 | −1 | 0.8923 | 4 | 0.8923 | 8 | 0.9312 | Human mRNA for NB thymosin |
| | | | | | | | | beta |
| 3 | Hs.1869 | 1 | 0.8878 | 7 | 0.8878 | 7 | 0.9351 | Human phosphoglucomutase 1 |
| | | | | | | | | (PGM1) mRNA |
| 4 | Hs.81874 | −1 | 0.8787 | 8 | 0.8787 | 20 | 0.9091 | Human microsomal glutathione S- |
| | | | | | | | | transferase (GST-II) mRNA |
| 5 | Hs.44481 | 1 | 0.8764 | 10 | 0.8764 | 5 | 0.9481 | Human forkhead protein FREAC-2 |
| | | | | | | | | mRNA |
| 6 | Hs.211933 | 1 | 0.8753 | 12 | 0.8753 | 3 | 0.9597 | Human (clones HT-[125 |
| 7 | Hs.155597 | 1 | 0.8617 | 13 | 0.8617 | 4 | 0.9494 | Human adipsin/complement factor |
| | | | | | | | | D mRNA |
| 8 | Hs.170328 | 1 | 0.8515 | 17 | 0.8515 | 28 | 0.8779 | Human moesin mRNA |
| 9 | Hs.82124 | 1 | 0.8424 | 21 | 0.8424 | 25 | 0.8896 | Human laminin B1 chain mRNA |
| 10 | Hs.76224 | 1 | 0.8424 | 22 | 0.8424 | 14 | 0.9195 | Human extracellular protein (S1-5) |
| | | | | | | | | mRNA |
| 11 | Hs.245188 | 1 | 0.8367 | 24 | 0.8367 | 6 | 0.9377 | Human tissue inhibitor of |
| | | | | | | | | metalloproteinases-3 mRNA |
| 12 | Hs.202097 | 1 | 0.8311 | 25 | 0.8311 | 56 | 0.8468 | Human procollagen C-proteinase |
| | | | | | | | | enhancer protein (PCOLCE) |
| | | | | | | | | mRNA |
| 13 | Hs.171862 | 1 | 0.8311 | 26 | 0.8311 | 38 | 0.8636 | Human guanylate binding protein |
| | | | | | | | | isoform II (GBP-2) mRNA |
| 14 | Hs.71622 | 1 | 0.8265 | 27 | 0.8265 | 24 | 0.8922 | Human SWI/SNF complex 60 KDa |
| | | | | | | | | subunit (BAF60c) mRNA |
| 15 | Hs.74615 | 1 | 0.822 | 28 | 0.822 | 51 | 0.8506 | Human platelet-derived growth |
| | | | | | | | | factor receptor alpha (PDGFRA) |
| | | | | | | | | mRNA |
| 16 | Hs.56045 | 1 | 0.8152 | 31 | 0.8152 | 1 | 0.9857 | Human mRNA for stac |
| 17 | Hs.78909 | 1 | 0.8143 | 16 | 0.8537 | 112 | 0.8143 | Human Tis11d gene |
| 18 | Hs.155585 | 1 | 0.8104 | 20 | 0.8458 | 126 | 0.8104 | Human transmembrane receptor |
| | | | | | | | | (ror2) mRNA |
| 19 | Hs.2799 | 1 | 0.8073 | 38 | 0.8073 | 53 | 0.8481 | Human link protein mRNA |
| 20 | Hs.237356 | 1 | 0.805 | 39 | 0.805 | 22 | 0.8961 | Human intercrine-alpha (hIRH) |
| | | | | | | | | mRNA |
| 21 | Hs.195850 | 1 | 0.8013 | 30 | 0.8175 | 146 | 0.8013 | Human keratin type II (58 kD) |
| | | | | | | | | mRNA |
| 22 | Hs.78913 | 1 | 0.8005 | 41 | 0.8005 | 31 | 0.874 | Human G protein-coupled receptor |
| | | | | | | | | V28 mRNA |
| 23 | Hs.172471 | 1 | 0.7987 | 23 | 0.8401 | 152 | 0.7987 | Homo sapiens (clone hKvBeta3) |
| | | | | | | | | K+ channel beta subunit mRNA |
| 24 | Hs.78089 | −1 | 0.7971 | 43 | 0.7971 | 45 | 0.8545 | Human fetus brain mRNA for |
| | | | | | | | | vacuolar ATPase |
| 25 | Hs.51299 | −1 | 0.7959 | 45 | 0.7959 | 27 | 0.8844 | Human nuclear-encoded |
| | | | | | | | | mitochondrial NADH-ubiquinone |
| | | | | | | | | reductase 24 Kd subunit 26 |
| | | | | | | | | Hs.83383 0.7948 37 0.8073 |
| 27 | Hs.10526 | 1 | 0.7948 | 5 | 0.89 | 163 | 0.7948 | Human smooth muscle LIM protein |
| | | | | | | | | (h-SmLIM) mRNA |
| 28 | Hs.2090 | 1 | 0.7937 | 49 | 0.7937 | 82 | 0.8299 | Human prostaglandin E2 receptor |
| | | | | | | | | mRNA |
| 29 | Hs.155591 | 1 | 0.7935 | 40 | 0.8005 | 165 | 0.7935 | Human forkhead protein FREAC-1 |
| | | | | | | | | mRNA |
| 30 | Hs.75111 | 1 | 0.7922 | 34 | 0.8118 | 168 | 0.7922 | Human cancellous bone osteoblast |
| | | | | | | | | mRNA for serin protease with 31 |
| | | | | | | | | Hs.76780 1 |
| 32 | Hs.153322 | 1 | 0.7902 | 54 | 0.7902 | 13 | 0.9221 | Human mRNA for phospholipase C |
| 33 | Hs.74566 | 1 | 0.7896 | 15 | 0.8549 | 172 | 0.7896 | Human mRNA for |
| | | | | | | | | dihydropyrimidinase related |
| | | | | | | | | protein-3 |
| 34 | Hs.0 | 1 | 0.7896 | 32 | 0.8141 | 173 | 0.7896 | Human CX3C chemokine precursor |
| 35 | Hs.149923 | −1 | 0.7868 | 57 | 0.7868 | 86 | 0.8286 | Human X box binding protein-1 |
| | | | | | | | | (XBP-1) mRNA |
| 36 | Hs.62661 | 1 | 0.7844 | 19 | 0.8458 | 185 | 0.7844 | Human guanylate binding protein |
| | | | | | | | | isoform I (GBP-2) mRNA |
| 37 | Hs.81412 | 1 | 0.7818 | 52 | 0.7914 | 191 | 0.7818 | Human mRNA for KIAA0188 gene |
| 38 | Hs.79914 | 1 | 0.78 | 61 | 0.78 | 48 | 0.8532 | Human lumican mRNA |
| 39 | Hs.0 | 1 | 0.7792 | 44 | 0.7959 | 198 | 0.7792 | Homo sapiens growth-arrest- |
| | | | | | | | | specific protein (gas) mRNA |
| 40 | Hs.151242 | 1 | 0.7789 | 62 | 0.7789 | 10 | 0.9312 | Human plasma protease (C1) |
| | | | | | | | | inhibitor mRNA |
| 41 | Hs.81071 | 1 | 0.7766 | 63 | 0.7766 | 19 | 0.9104 | Human extracellular matrix protein |
| | | | | | | | | 1 (ECM1) mRNA |
| 42 | Hs.1827 | 1 | 0.7755 | 65 | 0.7755 | 30 | 0.874 | Human nerve growth factor |
| | | | | | | | | receptor mRNA |
| 43 | Hs.171731 | 1 | 0.7753 | 6 | 0.8889 | 207 | 0.7753 | Human RACH1 (RACH1) mRNA |
| 44 | Hs.19368 | 1 | 0.7721 | 67 | 0.7721 | 34 | 0.8714 | Human matrilin-2 precursor mRNA |
| 45 | Hs.85146 | 1 | 0.7714 | 60 | 0.7823 | 214 | 0.7714 | Human erythroblastosis virus |
| | | | | | | | | oncogene homolog 2 (ets-2) mRNA |
| 46 | Hs.79059 | 1 | 0.771 | 68 | 0.771 | 92 | 0.8247 | Human transforming growth factor- |
| | | | | | | | | beta type III receptor (TGF-beta) mRNA |
| 47 | Hs.79226 | 1 | 0.7687 | 70 | 0.7687 | 11 | 0.926 | Human FEZ1 mRNA |
| 48 | Hs.27311 | −1 | 0.7662 | 36 | 0.8107 | 231 | 0.7662 | Human transcription factor SIM2 |
| | | | | | | | | long form mRNA |
| 49 | Hs.76688 | 1 | 0.7642 | 73 | 0.7642 | 60 | 0.8442 | Human carboxylesterase mRNA |
| 50 | Hs.155560 | −1 | 0.7623 | 74 | 0.7642 | 237 | 0.7623 | Homo sapiens integral membrane |
| | | | | | | | | protein |

There were only 17 BPH samples in the first study and only 10 in the second study. Hence, the pvalues obtained are not as good. Further, in the 2001 study, very few non-tumor samples are not BPH: 8 NL, 1 stroma, 3 Dysplasia. Therefore, the gene selection from the 2001 study samples is biased toward finding genes that separate well tumor vs. BPH and ignore the other controls.

As before, one dataset was used as training set and the other as test set, then the two datasets were swapped. This time, we get significantly better results by training on the study 1 data and testing on the study0 data. This can be explained by the fact that the first study included very few control samples other than BPH, which biases the feature selection.

Training on the 2003 study and testing on the 2001 study for 10 features yields about 10% error. This is not as good as the results obtained by cross-validation, where there was only one error, but still quite reasonable. Lesser results using an independent test set were expected since there are only 10 BPH samples in the 2003 study.

When the features are selected with the samples of the 2001 study, the normal samples are grouped with BPH in the 2003 study, even though the goal was to find genes separating BPH from all others. When the features are selected with the 2003 study samples, the BPH samples of study 0 are not well separated.

In conclusion, it was not obvious that there would be agreement between the genes selected using two independent studies that took place at different times using different arrays. Nonetheless, there was a significant overlap in the genes selected. Further, by training with the data from one study and testing on the data from the other good classification performances were obtained both for the tumor vs. others and the BPH vs. others separations (around 10% error). To obtain these results, the gene set was limited to only 2000 genes. There may be better candidates in the genes that were discarded, however, the preference was for increased confidence in the genes that have been validated by several studies.

### Example 7

### BPH Study

The training set used was the 2003 dataset in previous examples (Table 12). The test set was, the 2001 dataset (Table 39). The probes on the two array types were matched according to “Gene ID” numbers and descriptions, producing 2346 common genes, matched with confidence.

The training data were normalized first by the expression of the reference housekeeping gene ACTB. The resulting matrix was used to compute fold change and average expression magnitude. For computing other statistics and performing machine learning experiments, both the training data and the test data separately underwent the following preprocessing: take the log to equalize the variances; standardize the columns and then the lines twice; take the tanh to squash the resulting values.

The genes were ranked by AUC (area under the ROC curve), as a single gene filter criterion. The corresponding p values (pval) and false discovery rates (FDR) were computed to assess the statistical significance of the findings. In the resulting table, the genes were ranked by p value using training data only. The false discovery rate was limited to 0.01. This resulted in 120 genes. The top 50 genes for BPH are listed in Table 42 below.

TABLE 42

| | Unigene | | | | | | | | | |
|---|---|---|---|---|---|---|---|---|---|---|
| Num | ID (Hs.) | AUC | pval | FDR | Fisher | Pearson | FC | Mag | tAUC | Description |
| 5309 | 100431 | 0.9961 | 3.80E−07 | 0.0085 | 2.77 | 0.07 | 23.22 | 0.029 | | Homo sapiens small |
| | | | | | | | | | | inducible cytokine B |
| | | | | | | | | | | subfamily (Cys-X-Cys |
| | | | | | | | | | | motif) (CXCL13) |
| 3480 | 79389 | 0.9922 | 4.70E−07 | 0.0053 | 3.56 | 0.25 | 3.7 | 0.066 | | Homo sapiens nel |
| | | | | | | | | | | (chicken)-like 2 |
| | | | | | | | | | | (NELL2) |
| 5810 | 56045 | 0.9818 | 8.20E−07 | 0.0061 | 1.45 | 0.44 | 1.28 | 0.024 | 0.805 | Homo sapiens src |
| | | | | | | | | | | homology three (SH3) |
| | | | | | | | | | | and cysteine rich domain |
| | | | | | | | | | | (STAC) |
| 17802 | 3807 | 0.9818 | 8.20E−07 | 0.0046 | 2.06 | 0.45 | 2.15 | 0.097 | | Homo sapiens FXYD |
| | | | | | | | | | | domain-containing ion |
| | | | | | | | | | | transport regulator 6 |
| | | | | | | | | | | (FXYD6) |
| 4843 | 75774 | 0.0195 | 8.80E−07 | 0.0039 | 1.65 | 0.48 | 0.25 | 0.074 | | Homo sapiens |
| | | | | | | | | | | thrombospondin 4 |
| | | | | | | | | | | (THBS4) |
| 3063 | 79732 | 0.9792 | 9.40E−07 | 0.0035 | 0.98 | 0.25 | 1.95 | 0.079 | | Human DNA sequence |
| | | | | | | | | | | from clone CTA-941F9 |
| | | | | | | | | | | on chromosome 22q13 |
| | | | | | | | | | | Contains the 3 end of the |
| | | | | | | | | | | FBLN1 gene for Fibulin |
| | | | | | | | | | | 1 isoforms B (FBLN1) |
| 5497 | 1104 | 0.9779 | 1.00E−06 | 0.0032 | 1.33 | 0.03 | 10.99 | 0.42 | | Human DNA sequence |
| | | | | | | | | | | from clone RP1-181C24 |
| | | | | | | | | | | on chromosome 6p11.1- |
| | | | | | | | | | | 12.2. Contains the 3 end |
| | | | | | | | | | | of the BMP5 gene for |
| | | | | | | | | | | bone morphogenetic |
| | | | | | | | | | | protein 5 |
| 5498 | 1104 | 0.9688 | 1.60E−06 | 0.0045 | 1.45 | 0.17 | 2.58 | 0.75 | | Homo sapiens bone |
| | | | | | | | | | | morphogenetic protein 5 |
| | | | | | | | | | | (BMP5) |
| 5715 | 89791 | 0.9688 | 1.60E−06 | 0.004 | 0.84 | 0.07 | 2.92 | 0.97 | | Homo sapiens wingless- |
| | | | | | | | | | | type MMTV integration |
| | | | | | | | | | | site family member 2 |
| | | | | | | | | | | (WNT2) |
| 9897 | 26468 | 0.9662 | 1.80E−06 | 0.0041 | 1.64 | 0.05 | 1.63 | 0.0015 | | Homo sapiens mRNA |
| | | | | | | | | | | for XllL |
| 19651 | 16026 | 0.9649 | 2.00E−06 | 0.004 | 0.99 | 0.21 | 1.28 | 0.0019 | | Homo sapiens |
| | | | | | | | | | | hypothetical protein |
| | | | | | | | | | | FLJ23191 (FLJ23191) |
| 1217 | 245188 | 0.9623 | 2.20E−06 | 0.0042 | 1.47 | 0.53 | 1.33 | 0.00032 | | Hs.245188 tissue |
| | | | | | | | | | | inhibitor of |
| | | | | | | | | | | metalloproteinase 3 |
| | | | | | | | | | | (Sorsby fundus |
| | | | | | | | | | | dystrophy; pseudo- |
| | | | | | | | | | | inflammatory) (TIMP3) |
| 5631 | 95420 | 0.961 | 2.40E−06 | 0.0041 | 0.8 | 0.3 | 2.16 | 0.0007 | | Homo sapiens JM27 |
| | | | | | | | | | | protein (JM27) |
| 11301 | 211933 | 0.961 | 2.40E−06 | 0.0038 | 1.23 | 0.4 | 2.68 | 0.0012 | 0.8696 | Human (clones HT-125) |
| | | | | | | | | | | (COL4A2) |
| 3254 | 81256 | 0.9597 | 2.50E−06 | 0.0038 | 1.03 | 0.19 | 2.22 | 0.00092 | | Homo sapiens S100 |
| | | | | | | | | | | calcium-binding protein |
| | | | | | | | | | | A4 (calcium protein |
| 3399 | 155939 | 0.9571 | 2.90E−06 | 0.004 | 0.82 | 0.33 | 1.13 | 0.00024 | | Homo sapiens inositol |
| | | | | | | | | | | polyphosphate-5- |
| | | | | | | | | | | phosphatase |
| 3490 | 101850 | 0.9571 | 2.90E−06 | 0.0038 | 1.11 | 0.28 | 1.57 | 0.0003 | 0.7517 | Homo sapiens retinol- |
| | | | | | | | | | | binding protein 1 |
| 20879 | 0 | 0.9558 | 3.10E−06 | 0.0038 | 1.16 | 0.31 | 1.39 | 0.0012 | | Homo sapiens G protein |
| | | | | | | | | | | coupled receptor |
| | | | | | | | | | | interacting protein |
| 3311 | 154103 | 0.0481 | 3.80E−06 | 0.0044 | 1.6 | 0.6 | 0.19 | 0.001 | | Homo sapiens LIM |
| | | | | | | | | | | protein (similar to rat |
| | | | | | | | | | | protein kinase C-binding |
| | | | | | | | | | | enigma) (LIM) |
| 9713 | 77202 | 0.9494 | 4.30E−06 | 0.0048 | 1 | 0.28 | 1.32 | 0.0013 | | Homo sapiens protein |
| | | | | | | | | | | kinase C beta-II type |
| | | | | | | | | | | (PRKCB1) mRNA |
| 1219 | 245188 | 0.9481 | 4.60E−06 | 0.0048 | 1.11 | 0.46 | 1.67 | 0.00045 | | Homo sapiens tissue |
| | | | | | | | | | | inhibitor of |
| | | | | | | | | | | metalloproteinase 3 |
| | | | | | | | | | | (Sorsby fundus |
| | | | | | | | | | | dystrophy |
| 6443 | 44481 | 0.9481 | 4.60E−06 | 0.0046 | 1.72 | 0.11 | 2.49 | 0.00046 | 0.8753 | Homo sapiens forkhead |
| | | | | | | | | | | box F2 (FOXF2) |
| 3970 | 31720 | 0.9429 | 5.90E−06 | 0.0057 | 0.98 | 0.39 | 1.34 | 0.00029 | | Homo sapiens |
| | | | | | | | | | | hephaestin (HEPH) |
| 5449 | 155597 | 0.9429 | 5.90E−06 | 0.0054 | 0.65 | 0.41 | 2.36 | 0.00062 | 0.89 | Homo sapiens D |
| | | | | | | | | | | component of |
| | | | | | | | | | | complement (adipsin) |
| | | | | | | | | | | (DF) |
| 16714 | 306913 | 0.0571 | 5.90E−06 | 0.0052 | 1.34 | 0.55 | 0.12 | 0.0035 | | Homo sapiens cDNA: |
| | | | | | | | | | | FLJ23564 fis |
| 20655 | 10235 | 0.9416 | 6.20E−06 | 0.0054 | 0.79 | 0.41 | 3.11 | 0.015 | | Homo sapiens |
| | | | | | | | | | | chromosome 5 open |
| | | | | | | | | | | reading frame 4 |
| | | | | | | | | | | (C5ORF4) |
| 20091 | 44208 | 0.0584 | 6.20E−06 | 0.0052 | 1.04 | 0.37 | 0.2 | 0.017 | | Homo sapiens |
| | | | | | | | | | | hypothetical protein |
| | | | | | | | | | | FLJ23153 (FLJ23153) |
| 1856 | 79732 | 0.9403 | 6.60E−06 | 0.0053 | 0.8 | 0.36 | 3.55 | 0.063 | | Homo sapiens fibulin 1 |
| | | | | | | | | | | (FBLN1) |
| 1216 | 245188 | 0.939 | 7.10E−06 | 0.0054 | 0.81 | 0.38 | 1.85 | 0.00029 | 0.8628 | Hs.245188 tissue |
| | | | | | | | | | | inhibitor of |
| | | | | | | | | | | metalloproteinase 3 |
| | | | | | | | | | | (Sorsby fundus |
| | | | | | | | | | | dystrophy; |
| | | | | | | | | | | pseudoinflammatory) |
| 4779 | 284122 | 0.939 | 7.10E−06 | 0.0053 | 1.25 | 0.52 | 3.95 | 0.00033 | | Homo sapiens Wnt |
| | | | | | | | | | | inhibitory factor-1 (WIF- |
| | | | | | | | | | | 1) |
| 19044 | 76461 | 0.939 | 7.10E−06 | 0.0051 | 1.52 | 0.43 | 10.31 | 0.0014 | | Homo sapiens retinol- |
| | | | | | | | | | | binding protein 4 |
| 3416 | 43697 | 0.9377 | 7.50E−06 | 0.0052 | 0.95 | 0.29 | 1.59 | 0.4 | | Homo sapiens ets variant |
| | | | | | | | | | | gene 5 (ets-related |
| | | | | | | | | | | molecule) (ETV5) |
| 9469 | 5378 | 0.9377 | 7.50E−06 | 0.0051 | 1.14 | 0.35 | 1.44 | 0.39 | | Homo sapiens mRNA |
| | | | | | | | | | | for KIAA0762 protein |
| 9201 | 5422 | 0.9364 | 8.00E−06 | 0.0053 | 0.58 | 0.36 | 1.54 | 0.44 | | Hs.5422 glycoprotein |
| | | | | | | | | | | M6B |
| 2037 | 1869 | 0.9351 | 8.50E−06 | 0.0054 | 0.64 | 0.37 | 1.06 | 0.47 | 0.9433 | Homo sapiens |
| | | | | | | | | | | phosphoglucomutase 1 |
| | | | | | | | | | | (PGM1) |
| 4078 | 18676 | 0.9351 | 8.50E−06 | 0.0053 | 1.15 | 0.44 | 1.32 | 0.73 | | Homo sapiens sprouty |
| | | | | | | | | | | (Drosophila) homolog 2 |
| | | | | | | | | | | (SPRY2) |
| 3310 | 154103 | 0.0649 | 8.50E−06 | 0.0051 | 1.23 | 0.58 | 0.14 | 0.71 | | Hs.154103 LIM protein |
| | | | | | | | | | | (similar to rat protein |
| | | | | | | | | | | kinase C-binding |
| | | | | | | | | | | enigma) |
| 2756 | 83429 | 0.0662 | 9.10E−06 | 0.0053 | 0.92 | 0.24 | 0.29 | 0.0058 | 0.3152 | Homo sapiens Apo-2 |
| | | | | | | | | | | ligand mRNA |
| 4998 | 78061 | 0.9338 | 9.10E−06 | 0.0052 | 0.74 | 0.23 | 1.53 | 0.012 | | Homo sapiens |
| | | | | | | | | | | transcription factor 21 |
| | | | | | | | | | | (TCF21) |
| 14516 | 162209 | 0.0675 | 9.70E−06 | 0.0054 | 1.14 | 0.56 | 0.2 | 0.0043 | | Homo sapiens mRNA |
| 12549 | 169965 | 0.9325 | 9.70E−06 | 0.0052 | 0.47 | 0.17 | 1.49 | 1.5 | | Hs.169965 chimerin |
| | | | | | | | | | | (chimaerin) 1 |
| 19274 | 100890 | 0.9325 | 9.70E−06 | 0.0051 | 0.75 | 0.38 | 1.54 | 0.035 | | Homo sapiens candidate |
| | | | | | | | | | | mediator of the p53- |
| | | | | | | | | | | dependent G2 arrest |
| | | | | | | | | | | (REPRIMO) |
| 19841 | 6510 | 0.9325 | 9.70E−06 | 0.005 | 0.43 | 0.28 | 1.61 | 0.063 | | Homo sapiens |
| | | | | | | | | | | thyrotropin-releasing |
| | | | | | | | | | | hormone degrading |
| | | | | | | | | | | ectoenzyme (TRHDE) |
| 12337 | 7780 | 0.0701 | 1.10E−05 | 0.0055 | 2.11 | 0.69 | 0.29 | 0.035 | | Hs.7780 Homo sapiens |
| | | | | | | | | | | mRNA; cDNA |
| | | | | | | | | | | DKFZp564A072 (from |
| | | | | | | | | | | clone DKFZp564A072) |
| 1055 | 151242 | 0.9299 | 1.10E−05 | 0.0054 | 0.57 | 0.16 | 1.64 | 0.11 | 0.8141 | Homo sapiens serine (or |
| | | | | | | | | | | cysteine) proteinase |
| | | | | | | | | | | inhibitor |
| 9574 | 85112 | 0.9286 | 1.20E−05 | 0.0056 | 0.7 | 0.35 | 1.65 | 0.13 | | Hs.85112 insulin-like |
| | | | | | | | | | | growth factor 1 |
| | | | | | | | | | | (somatomedin C) |
| 15456 | 25220 | 0.9286 | 1.20E−05 | 0.0055 | 0.9 | 0.36 | 1.28 | 0.36 | | Homo sapiens mRNA |
| | | | | | | | | | | for KIAA0609 protein |
| 18622 | 43080 | 0.9286 | 1.20E−05 | 0.0054 | 0.83 | 0.39 | 1.2 | 0.39 | | Homo sapiens platelet |
| | | | | | | | | | | derived growth factor C |
| | | | | | | | | | | (PDGFC) |
| 5819 | 75652 | 0.9273 | 1.20E−05 | 0.0056 | 1 | 0.42 | 1.26 | 1.3 | 0.7132 | Homo sapiens |
| | | | | | | | | | | glutathione S-transferase |
| | | | | | | | | | | M5 (GSTM5) |
| 8440 | 1408 | 0.9273 | 1.20E−05 | 0.0055 | 0.73 | 0.52 | 1.45 | 1.4 | | Homo sapiens |
| | | | | | | | | | | endothelin 3 (EDN3) |

The definitions of the statistics used in the ranking are provided in Table 43.

TABLE 43

| Statistic | Description |
|---|---|
| AUC | Area under the ROC curve of individual genes, using training tissues. The ROC curve |
| | (receiver operating characteristic) is a plot of the sensitivity (error rate of the “positive” |
| | class, i.e., the BPH tissue error rate) vs. the specificity (error rate of the “negative” |
| | class, here non-BPH tissues. Insignificant genes have anAUC close to 0.5. Genes with |
| | an AUC closer to one are overexpressed in cancer. Genes with an AUC closer to zero are |
| | underexpressed. |
| pval | Pvalue of the AUC, used as a test statistic to test the equality of the median of the two |
| | population (BPH and non-BPH.) The AUC is the Mann-Withney statistic. The test is |
| | equivalent to the Wilcoxon rank sum test. Small pvalues shed doubt on the null |
| | hypothesis of equality of the medians. Hence smaller values are better. To account to the |
| | multiple testing the pvalue may be Bonferroni corrected by multiplying it by the number |
| | of genes 7129. |
| FDR | False discovery rate of the AUC ranking. An estimate of the fraction of insignificant |
| | genes in the genes ranking higher than a given gene. It is equal the pvalue multiplied by |
| | the number of genes 7129 and divided by the rank. |
| Fisher | Fisher statistic characterizing the multiclass discriminative power for the histological |
| | classes (normal, BPH, dysplasia, grade 3, and grade 4.) The Fisher statistic is the ratio of |
| | the between-class variance to the within-class variance. Higher values indicate better |
| | discriminative power. The Fisher statistic can be interpreted as a signal to noise ratio. It |
| | is computed with training data only. |
| Pearson | Pearson correlation coefficient characterizing “disease progression”, with histological |
| | classes coded as 0 = normal, 1 = BPH, 2 = dysplasia, 3 = grade 3, and 4 = grade 4.) A value |
| | close to 1 indicates a good correlation with disease progression. |
| FC | Fold change computed as the ratio of the average BPH expression values to the |
| | avarage of the other expression values. It is computed with training data only. A value |
| | near one indicates an insignificant gene. A large value indicates a gene overexpressed in |
| | BPH; a small value an underexpressed gene. |
| Mag | Gene magnitude. The average of the largest class expression value (BPH or other) |
| | relative to that of the ACTB housekeeping gene. It is computed with training data only. |
| tAUC | AUC of the genes matched by probe and or description in the test set. It is computed |
| | with test data only, hence not all genes have a tAUC. |

The 120 top ranking genes using the AUC criterion, satisfy FDR<=0.01, i.e. including less than 1% insignificant genes. Note that the expression values have undergone the preprocessing described above, including taking the log and standardizing the genes.

TABLE 45

| SEQ ID NO. | Gene Symbol | Unigene | Description |
|---|---|---|---|
| 1 | CXCL13 | Hs.100431 | Small inducible cytokine B subfamily (Cys-X-Cys |
| | | | motif); member 13 (B-cell chemoattractant) (SCYB13) |
| 2 | NELL2 | Hs.79389 | Nel (chicken)-like 2 (NELL2) |
| 3 | SH3 | Hs.56045 | Src homology three (SH3) and cysteine rich domain |
| | | | (STAC) |
| 4 | FBLN1 | Hs.79732 | Contains the 3 end of the FBLN1 gene for Fibulin 1 |
| | | | isoforms B; C and D |
| 5 | BMP5 | Hs.1104 | Bone morphogenetic protein 5 (BMP5) |
| 6 | WNT2 | Hs.89791 | Wingless-type MMTV integration site family member 2 |
| | | | (WNT2) |
| 7 | X11L | Hs.26468 | Amyloid beta (A4) precursor protein -binding; family |
| | | | A; member 2 (X11-like) |
| 8 | TIMP3 | Hs.245188 | T issue inhibitor of metalloproteinase 3 (Sorsby fundus |
| | | | dystrophy; pseudoinflammatory) |
| 9 | JM27 | Hs.95420 | JM27 protein |
| 10 | COL4A2 | Hs.211933 | Alpha-2 type IV collagen (COL4A2) |
| 11 | GATA-6 | Hs.50924 | GATA-binding protein 6 |
| 12 | CXCL12 | Hs.237356 | Chemokine (C-X-C motif) ligand 12. Intercrine-alpha |
| | | | (hIRH) Stromal cell-derived factor 1 (SDF1) |
| 13 | IGF-2 | Hs.251664 | Insulin -like growth factor II (IGF -2) |
| 14 | TGF-beta3 | Hs.2025 | Transforming growth factor-beta 3 (TGF -beta3) |
| 15 | PTGDS | Hs.8272 | Prostaglandin D2 synthase (21 kD; brain) (PTGDS) |

An investigation was performed to determine whether the genes are ranked similarly with training and test data. Because training and test data were processed by different arrays, this analysis was restricted to 2346 matched probes. This narrowed down the 120 genes previously selected with the AUC criterion to 23 genes. It was then investigated whether this selection corresponds to genes that also rank high when genes are ranked by the test data. Genes selected are found much faster than by chance. Additionally, 95% of the 23 genes selected with training data are similarly “oriented” (i.e. overexpressed or underexpressed in both datasets.

In some applications, it is important to select genes that not only have discriminative power, but are also salient, i.e. have a large fold change (FC) and a large average expression value of the most expressed category (Mag.) Some of the probes correspond to genes belonging to the same Unigene cluster. This adds confidence to the validity of these genes.

A predictive model is trained to make the separation BPH v.s. non-BPH using the available training data. Its performance is then assessed with the test data (consisting of samples collected at different times, processed independently and with a different microarray technology.) Because the arrays used to process the training and test samples are different, our machine learning analysis utilizes only the 2346 matched probes. To extend the validation to all the genes selected with the training data (including those that are not represented in the test arrays) the set of genes was narrowed down to those having a very low FDR on training data (FDR<=0.01.) In this way, the machine learning analysis indirectly validates all the selected genes.

As previously mentioned, the first step of this analysis was to restrict the gene set by filtering those genes with FDR<=0.01 in the AUC feature ranking obtained with training samples. The resulting 120 genes are narrowed down to 23 by “projecting” them on the 2346 probes common in training and test arrays.

Two feature selection strategies are investigated to further narrow down the gene selection: the univariate and multivariate methods. The univariate method, which consists in ranking genes according to their individual predictive power, is examplified by the AUC ranking. The multivariate method, which consists in selecting subsets of genes that together provide a good predictive power, is examplified by the recursive feature elimination (RFE) method. RFE consists in starting with all the genes and progressively eliminating the genes that are least predictive. (As explained above, we actually start with the set of top ranking AUC genes with FDR<=0.01.) We use RFE with a regularized kernel classifier analogous to a Support Vector Machine (SVM.)

For both methods (univariate and multivariate), the result is nested subsets of genes. Importantly, those genes are selected with training data only.

A predictive model (a classifier) is built by adjusting the model parameters with training data. The number of genes is varied by selecting gene subsets of increasing sizes following the previously obtained nested subset structure. The model is then tested with test data, using the genes matched by probe and description in the test arrays. The hyperparameters are adjusted by cross-validation using training data only. Hence, both feature selection and all the aspect of model training are performed on training data only.

As for feature selection, two different paradigms are followed: univariate and multivariate. The univariate strategy is examplified by the Naive Bayes classifier, which makes independence assumptions between input variables. The multivariate strategy is examplied by the regularized kernel classifier. Although one can use a multivariate feature selection with a univariate classifier and vive versa, to keep things simple, univariate feature selection and classifier methods were used together, and similarly for the multivariate approach.

Using training data only automatically identified 4 outliers which were removed from the rest of the analysis.

Performances were measured with the area under the ROC curve (AUC). The ROC curve plots sentivivity as a function of specificity. The optimal operatic point is application specific. The AUC provides a measure of accuracy independent of the choice of the operating point.

Both univariate and multivariate methods perform well. The error bars on test data are of the order of 0.04, and neither method outperforms the other significantly. There is an indication that the multivariate method (RFE/kernel classifier) might be better for a smaller number of features. This can be explained by the fact that RFE removes feature redundancy. In this example, the top 10 genes for the univariate method (AUC criterion) are {Hs.56045, Hs.211933, Hs.101850, Hs.44481, Hs.155597, Hs.1869, Hs.151242, Hs.83429, Hs.245188, Hs.79226,} and those selected by the multivariate method (RFE) are {Hs.44481, Hs.83429, Hs.101850, Hs.2388, Hs.211933, Hs.56045, Hs.81874, Hs.153322, Hs.56145, Hs.83551,}. Note that the AUC-selected genes are different from the top genes listed in Table 42 for 2 reasons: 1) only the genes matched with test array probes are considered (corresponding to genes having a tAUC value in the table) and 2) a few outlier samples were removed and the ranking was rerun.

### Example 8

### BPH Study #2

The training set used was the 2003 dataset in previous examples (Table 12). The test set was, the 2001 dataset (Table 39). The probes on the two array types were matched according to “Gene ID” numbers and descriptions, producing 2346 common genes, matched with confidence.

The training data were normalized first by the expression of the reference housekeeping gene ACTB. The resulting matrix was used to compute fold change and average expression magnitude. For computing other statistics and performing machine learning experiments, both the training data and the test data separately underwent the following preprocessing: take the log to equalize the variances; standardize the columns and then the lines twice; take the tanh to squash the resulting values.

The genes were ranked by AUC (area under the ROC curve), as a single gene filter criterion. The corresponding p values (pval) and false discovery rates (FDR) were computed to assess the statistical significance of the findings. In the resulting table, the genes were ranked by p value using training data only. Genes having a FDR lower than 0.01 in the 2003 dataset were retained for investigation. The set was further restricted to those genes having a fold change (FC) larger than 2. The AUC score was calculated for the genes in the 2001 dataset that have a match in the 2003 dataset. The two datasets were merged and an overall normalization was performed. The genes were then ranked according to AUC in the merged set, allowing genes with a FDR of less than 10⁻⁶ to be identified. Additional criteria that may be used include genes with a magnitude greater than 0.1 ACTB and genes that have a tAUC larger than 0.75.

Table 44 provides the results ranked by AUC, including the name of the expressed protein. The right-most column lists the corresponding probe set ID on the Affymetrix U133A GeneChip® microarray.

TABLE 44

| Num | Protein | Unigene | AUC | FDR | FC | Mag | tAUC | Description | Probe |
|---|---|---|---|---|---|---|---|---|---|
| 5309 | CXCL13 | Hs.100431 | 0.996 | 0.009 | 23.22 | 0.04 | | Small inducible cytokine B | 205242_at |
| | | | | | | | | subfamily (Cys-X-Cys motif); | |
| | | | | | | | | member 13 (B-cell | |
| | | | | | | | | chemoattractant) (SCYB13) | |
| 3480 | NELL2 | Hs.79389 | 0.992 | 0.005 | 3.7 | 0.05 | | Nel (chicken)-like 2 (NELL2) | 203413_at |
| 5810 | SH3 | Hs.56045 | 0.982 | 0.006 | 1.28 | 0.02 | 0.805 | Src homology three (SH3) and | 205743_at |
| | | | | | | | | cysteine rich domain (STAC) | |
| 3063 | FBLN1 | Hs.79732 | 0.979 | 0.003 | 1.95 | 0.06 | | Contains the 3 end of the | 202994_s_at |
| | | | | | | | | FBLN1 gene for Fibulin 1 | |
| | | | | | | | | isoforms B; C and D | |
| 5497 | BMP5 | Hs.1104 | 0.978 | 0.003 | 10.99 | 0 | | Contains the 3 end of the | 205430_at |
| | | | | | | | | BMP5 gene for bone | |
| | | | | | | | | morphogenetic protein 5 | |
| 5715 | WNT2 | Hs.89791 | 0.969 | 0.004 | 2.92 | 0.01 | | Wingless-type MMTV | 205648_at |
| | | | | | | | | integration site family member 2 | |
| | | | | | | | | (WNT2) | |
| 5498 | BMP5 | Hs.1104 | 0.969 | 0.004 | 2.58 | 0.02 | | Bone morphogenetic protein 5 | 205431_s_at |
| | | | | | | | | (BMP5) | |
| 9897 | X11L | Hs.26468 | 0.966 | 0.004 | 1.63 | 0 | | Amyloid beta (A4) precursor | 209871_s_at |
| | | | | | | | | protein -binding; family A; | |
| | | | | | | | | member 2 (X11-like) | |
| 1217 | TIMP3 | Hs.245188 | 0.962 | 0.004 | 1.33 | 0.03 | | T issue inhibitor of | 201148_s_at |
| | | | | | | | | metalloproteinase 3 (Sorsby | |
| | | | | | | | | fundus dystrophy; | |
| | | | | | | | | pseudoinflammatory) | |
| 5631 | JM27 | Hs.95420 | 0.961 | 0.004 | 2.16 | 0.16 | | JM27 protein | 205564_at |
| 11301 | COL4A2 | Hs.211933 | 0.961 | 0.004 | 2.68 | 0.01 | 0.87 | Alpha-2 type IV collagen | 211343_s_at |
| | | | | | | | | (COL4A2) | |
| 20879 | ZSIG37 | Hs.0 | 0.956 | 0.004 | 1.39 | 0.02 | | G protein coupled receptor | 220975_s_at |
| | | | | | | | | interacting protein; complement- | |
| | | | | | | | | c1q tumor necrosis factor-related | |
| | | | | | | | | (ZSIG37) | |
| 6443 | FOXF2 | Hs.44481 | 0.948 | 0.005 | 2.49 | 0.02 | 0.875 | Forkhead box F2 (FOXF2) | 206377_at |
| 1219 | TIMP3 | Hs.245188 | 0.948 | 0.005 | 1.67 | 0.09 | | T issue inhibitor of | 201150_s_at |
| | | | | | | | | metalloproteinase 3 (Sorsby | |
| | | | | | | | | fundus dystrophy; | |
| | | | | | | | | pseudoinflammatory) | |
| 5449 | Adipsin | Hs.155597 | 0.943 | 0.005 | 2.36 | 0.04 | 0.89 | D component of complement | 205382_s_at |
| | | | | | | | | (adipsin) (DF) | |
| 1856 | FBLN1 | Hs.79732 | 0.94 | 0.005 | 3.55 | 0.06 | | Fibulin 1 (FBLN1); transcript | 201787_at |
| | | | | | | | | variant C | |
| 4779 | WIF-1 | Hs.284122 | 0.939 | 0.005 | 3.95 | 0.02 | | Wnt inhibitory factor-1 (WIF-1 ) | 204712_at |
| 1216 | TIMP3 | Hs.245188 | 0.939 | 0.005 | 1.85 | 0.02 | 0.863 | T tissue inhibitor of | 201147_s_at |
| | | | | | | | | metalloproteinase 3 (Sorsby | |
| | | | | | | | | fundus dystrophy; | |
| | | | | | | | | pseudoinflammatory) | |
| 4998 | TCF21 | Hs.78061 | 0.934 | 0.005 | 1.53 | 0.02 | | T ranscription factor 21 (TCF21) | 204931_at |
| 19274 | REPRIMO | Hs.100890 | 0.932 | 0.005 | 1.54 | 0.02 | | Candidate mediator of the p53- | 219370_at |
| | | | | | | | | dependent G2 arrest (REPRIMO) | |
| 1055 | SERPING1 | Hs.151242 | 0.93 | 0.005 | 1.64 | 0.04 | 0.814 | Serine (or cysteine) proteinase | 200986_at |
| | | | | | | | | inhibitor; clade G (C1 inhibitor); | |
| | | | | | | | | member 1 (SERPING1) | |
| 18622 | PDGFC | Hs.43080 | 0.929 | 0.005 | 1.2 | 0.07 | | P latelet derived growth factor C | 218718_at |
| | | | | | | | | (PDGFC) | |
| 9574 | IGF1-like | Hs.85112 | 0.929 | 0.006 | 1.65 | 0.06 | | Insulin -like growth factor 1 | 209541_at |
| | | | | | | | | (somatomedin C) | |
| 6136 | EPHA3 | Hs.123642 | 0.926 | 0.006 | 1.79 | 0.04 | | Ephrin receptor EPHA3 | 206070_s_at |
| | | | | | | | | complete form (EPHA3) | |
| 11595 | Laminin B1 | Hs.0 | 0.925 | 0.006 | 1.39 | 0.01 | | Laminin B1 | 211651_s_at |
| 5195 | PTGS1/COX1 | Hs.88474 | 0.923 | 0.006 | 1.24 | 0.02 | | P rostaglandin -endoperoxide | 205128_x_at |
| | | | | | | | | synthase 1 (prostaglandin GH | |
| | | | | | | | | synthase and cyclooxygenase) | |
| | | | | | | | | (PTGS1) | |
| 1911 | EFEMP1 | Hs.76224 | 0.922 | 0.006 | 1.41 | 0.08 | 0.824 | EGF-containing fibulin -like | 201842_s_at |
| | | | | | | | | extracellular matrix protein 1 | |
| 10028 | GATA-6 | Hs.50924 | 0.921 | 0.006 | 2.09 | 0.01 | | GATA-binding protein 6 | 210002_at |
| 3061 | C7 | Hs.78065 | 0.921 | 0.006 | 2.53 | 0.09 | | Cmplement component 7 (C7) | 202992_at |
| 1138 | MMP2 | Hs.111301 | 0.916 | 0.007 | 1.89 | 0.05 | | Matrix metalloproteinase 2 | 201069_at |
| | | | | | | | | (gelatinase A, type IV | |
| | | | | | | | | collagenase) | |
| 3392 | COL5A1 | Hs.146428 | 0.914 | 0.007 | 1.71 | 0.01 | 0.418 | Collagen, type V, alpha 1 | 203325_s_at |
| 13911 | COL4A6 | Hs.408 | 0.913 | 0.007 | 1.28 | 0.02 | | Collagen, type IV, alpha 6 | 213992_at |
| 11607 | PTGDS | Hs.0 | 0.909 | 0.008 | 2.52 | 0.11 | | P rostaglandin D synthase | 211663_x_at |
| 3064 | FBLN1 | Hs.79732 | 0.909 | 0.008 | 1.91 | 0.07 | | Fibulin 1 (FBLN1); transcript | 202995_s_at |
| | | | | | | | | variant D | |
| 13211 | COL4A2 | Hs.159263 | 0.906 | 0.008 | 1.48 | 0.01 | | Collagen, type VI, alpha 2 | 213290_at |
| 20019 | T2-cadherin | Hs.92489 | 0.905 | 0.008 | 1.35 | 0.01 | | Cadherin 10, type 2 (T2- | 220115_s_at |
| | | | | | | | | cadherin) (CDH10) | |
| 9715 | CXCL12 | Hs.237356 | 0.903 | 0.009 | 1.36 | 0.03 | 0.814 | Chemokine (C-X-C motif) | 209687_at |
| | | | | | | | | ligand 12. Intercrine-alpha | |
| | | | | | | | | (hIRH) Stromal cell-derived | |
| | | | | | | | | factor 1 (SDF1) | |
| 11688 | PTGDS | Hs.0 | 0.903 | 0.008 | 2.12 | 0.3 | | P rostaglandin D2 synthase | 211748_x_at |
| 2478 | IGF-2 | Hs.251664 | 0.903 | 0.009 | 2 | 0.02 | | Insulin -like growth factor II | 202409_at |
| | | | | | | | | (IGF -2) | |
| 9775 | TGF-beta3 | Hs.2025 | 0.9 | 0.009 | 1.61 | 0.06 | 0.941 | T ransforming growth factor- | 209747_at |
| | | | | | | | | beta 3 (TGF -beta3) | |
| 11464 | BMP4 | Hs.68879 | 0.899 | 0.009 | 2.71 | 0 | | Bone morphogenetic protein 4 | 211518_s_at |
| | | | | | | | | (BMP -4) | |
| 8179 | PTGIS | Hs.0 | 0.897 | 0.01 | 1.49 | 0.03 | | P rostaglandin I2 (prostacyclin) | 208131_s_at |
| | | | | | | | | synthase (PTGIS) | |
| 10633 | LTBP -4S | Hs.85087 | 0.897 | 0.01 | 1.36 | 0.01 | | Latent transforming growth | 210628_x_at |
| | | | | | | | | factor-beta binding protein 4S | |
| 12113 | PTGDS | Hs.8272 | 0.896 | 0.01 | 2.05 | 0.27 | | P rostaglandin D2 synthase | 212187_x_at |
| | | | | | | | | (21 kD; brain) (PTGDS) | |
| 1303 | ILK | Hs.6196 | 0.895 | 0.01 | 1.2 | 0.07 | 0.738 | Integrin -linked kinase (ILK) | 201234_at |
| 13349 | COL4A1 | Hs.108885 | 0.895 | 0.01 | 1.5 | 0.07 | | Collagen; type VI; alpha 1 | 213428_s_at |
| 9389 | MBP1 | Hs.6059 | 0.878 | 0.013 | 1.51 | 0.03 | | P 53 binding protein 1 (MBP1) | 209356_x_at |
| 5925 | NGFR | Hs.1827 | 0.874 | 0.014 | 2.32 | 0 | 0.776 | Nerve growth factor receptor | 205858_at |
| | | | | | | | | (TNFR superfamily; member | |
| | | | | | | | | 16) (NGFR) | |
| 992 | Galectin6 | Hs.79339 | 0.87 | 0.015 | 1.65 | 0.08 | | Lectin; galactoside -binding; | 200923_at |
| | | | | | | | | soluble; 3 binding protein | |
| | | | | | | | | (galectin 6 binding protein) | |
| | | | | | | | | (LGALS3BP) | |
| 2076 | Nidogen | Hs.62041 | 0.851 | 0.021 | 1.62 | 0.01 | 0.67 | Nidogen (enactin) | 202007_at |
| 4798 | TGFBR3 | Hs.79059 | 0.836 | 0.025 | 1.04 | 0.02 | 0.755 | T ransforming growth factor; | 204731_at |
| | | | | | | | | beta receptor III (betaglycan; | |
| | | | | | | | | 300 kD) (TGFBR3) | |
| 5645 | ROR2 | Hs.155585 | 0.809 | 0.04 | 1.33 | 0.01 | 0.847 | Receptor tyrosine kinase-like | 205578_at |
| | | | | | | | | orphan receptor 2 (ROR2) | |
| 4840 | IL-11R | Hs.64310 | 0.806 | 0.042 | 0.97 | 0.01 | | Interleukin 11 receptor; alpha | 204773_at |
| | | | | | | | | (IL11RA) | |
| 2265 | DKK3 | Hs.4909 | 0.805 | 0.043 | 1.32 | 0.02 | | Dickkopf (Xenopus laevis) | 202196_s_at |
| | | | | | | | | homolog 3 (DKK3) | |
| 1043 | ACTA2 | Hs.195851 | 0.791 | 0.056 | 0.98 | 0.56 | | Actin, alpha 2, smooth muscle; | 200974_at |
| | | | | | | | | aorta (ACTA2) | |
| 2343 | ATTG2 | Hs.78045 | 0.774 | 0.074 | 1.12 | 0.49 | | Actin, gamma 2;, smooth | 202274_at |
| | | | | | | | | muscle, enteric (ACTG2) | |
| 13946 | P38IP | Hs.171185 | 0.774 | 0.073 | 1.37 | 0.12 | | T ranscription factor (p38 | 214027_x_at |
| | | | | | | | | interacting protein) | |
| 4489 | FGF2 | Hs.284244 | 0.758 | 0.095 | 1.13 | 0.01 | | Fibroblast growth factor 2 | 204422_s_at |
| | | | | | | | | (basic) (FGF2) | |
| 5220 | TNFRSF5 | Hs.25648 | 0.74 | 0.124 | 1.09 | 0.01 | | T umor necrosis factor receptor | 205153_s_at |
| | | | | | | | | superfamily; member 5 | |
| | | | | | | | | (TNFRSF5) | |
| 8448 | Catenin | Hs.166011 | 0.731 | 0.142 | 0.83 | 0.04 | | Catenin (cadherin -associated | 208407_s_at |
| | | | | | | | | protein); delta 1 (CTNND1) | |
| 12728 | NGFI-A | Hs.159223 | 0.717 | 0.17 | 0.94 | 0.01 | | NGFI-A binding protein 2 | 212803_at |
| | | | | | | | | (ERG1 binding protein 2) | |
| 893 | GSTP1 | Hs.226795 | 0.623 | 0.49 | 0.89 | 0.05 | | Glutathione S-transferase pi | 200824_at |
| | | | | | | | | (GSTP1) | |

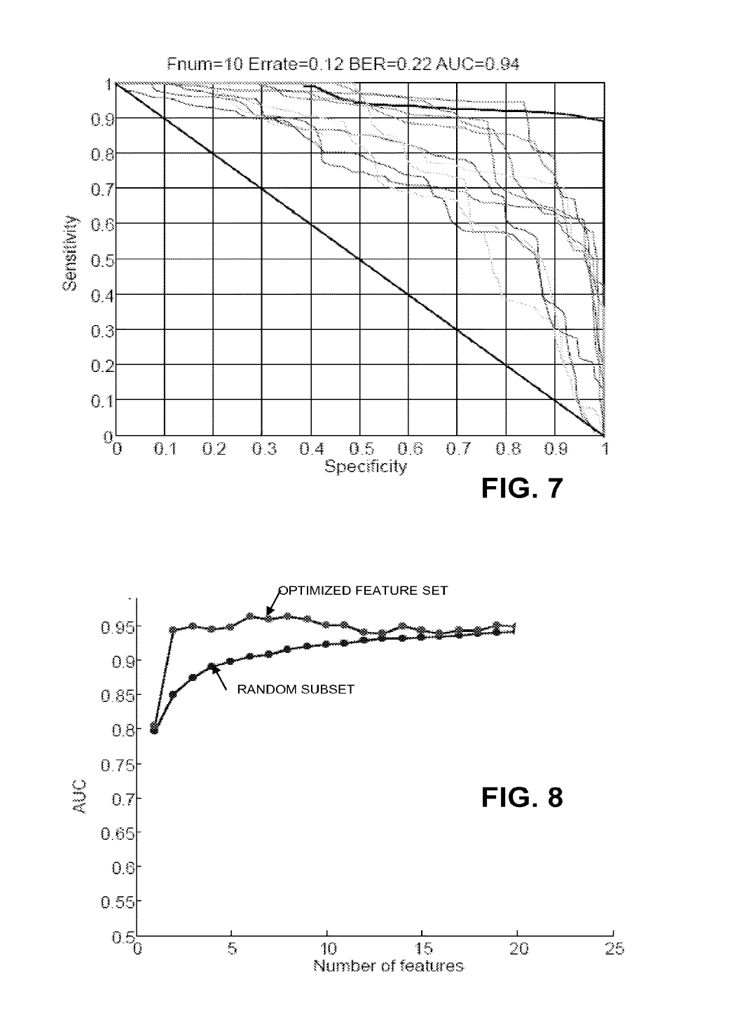

FIG. 7 shows the ROC curves for the 10 top ranking genes from Table 44 according to the AUC criterion, using the 2003 dataset for training and the 2001 dataset for testing, where the genes were identified using the training data, the classifier was trained using the training data, and the ROC curves were generated using the test data. Table 45 provides a cross-reference for 15 of the top ranking genes from Table 44 with their corresponding SEQ ID NO.

FIG. 8 shows the AUC for varying numbers of discriminative BPH genes. The lower curve is a plot of random combinations of the 23 genes present in both the training and test set that have a FDR<0.01 on the training set. The top ranking genes in ranked order produce the upper curve.

The most promising drug targets for treatment of BPH would be membrane receptor proteins, such as Her-2 in breast cancer (tyrosine kinases) ad/or cytoplasmic signaling proteins or enzymes, which control proliferation, or perhaps enzymes involved in blockin apoptosis transcription factors.

An interesting observation is that, while they are not listed in Table 44, the complete ranking results were searched for descriptions containing “PSA”. The highest ranks at which PSA appears were 6,749 and 9,486 out of the possible 22,283, with AUCs of 0.66 and 0.62, respectively.

A number of the genes identified in the study are involved in the Wnt (Wingless-INT) signaling pathway, and particularly the Wnt/TCF (T-cell factor) signaling pathway, which is associated with cell proliferation and differentiation, and is a highly conserved pathway. These genes include Hs.89791 (WNT2), Hs.284122 (WIF-1), Hs.78061 (TCF21), Hs.1104 (BMP5) and Hs.68879 (BMP4). Thus, it appears that one important mechanism of BPH is related to this pathway.

A second pathway that includes a number of the genes identified in the BPH study is the TGF (tumor growth factor), indicating the BPH is in some way related an inflammatory response. The genes within the TGF pathway include Hs.100431 (SCYB13/CXCL13), Hs.37356 (CXCL12), Hs.2025 (TGF-beta3), Hs.50924 (GATA-6), Hs.8272 (PTGD5), Hs.83429 (TNFSF10). The first five of these genes are overexpressed, some strongly, in BPH, while the last gene is underexpressed in BPH. These genes also intervene in the MAPK cell survival pathway. Other genes that are overexpressed in BPH that may be related to the TGF pathway include Hs.1104 (BMP5), Hs.68879 (BMP4), Hs.251664 (IGF-2), and Hs.85087 (LTBP4).

Although chronic inflammation (prostatitis) have not been reported as a risk factor for BPH, inflammatory or pseudo-inflammatory response seems to be activated in BPH. Thus, genes identified in the study as overexpressed in BPH may be more indicative of a symptom than a cause.

The present invention comprises biomarkers for screening, predicting and monitoring benign prostate hyperplasia that have been identified using SVM and other classifiers according to specified criteria. The availability of such biomarkers will lead to development of tests that can be used to detect and monitor BPH in men using tissue, semen or, preferably, serum samples, to reduce unnecessary prostatectomies and other surgical procedures resulting from the inability of current PSA-based diagnostics to distinguish between BPH and cancers that warrant more aggressive treatment.

Alternative embodiments of the present invention will become apparent to those having ordinary skill in the art to which the present invention pertains. Such alternate embodiments are considered to be encompassed within the spirit and scope of the present invention. Accordingly, the scope of the present invention is to be limited solely by the appended claims, which are supported by the foregoing exemplary embodiments of the invention.

1. A method for distinguishing between benign prostate hyperplasia (BPH) and non-BPH in a biological sample comprising screening for overexpression of at least one gene selected from the group of genes listed in Table 42 having:
an area under the curve (AUC) value of greater than 0.90;
a false discover rate (FDR) of less than 0.01; and
a value for fold change (FC) of greater than 3.

2. The method of claim 1, comprising detecting the expression level of any combination of two or more genes, or the respective protein products thereof, selected from the group consisting of CXLCL13 (SEQ ID NO. 1), NELL2 (SEQ ID NO. 2), SH3 (SEQ ID NO. 3), FBLN1 (SEQ ID NO. 4), BMP5 (SEQ ID NO. 5), WNT2 (SEQ ID NO. 6), X11L (SEQ. ID NO. 7), TIMP3 (SEQ ID NO. 8), JM27 (SEQ ID NO. 9) and COL4A2 (SEQ ID NO. 10).

3. The method of claim 1, wherein at least one gene is involved in the Wnt pathway.

4. The method of claim 3, wherein the expression levels of BMP5 (SEQ ID NO. 5) and WNT2 (SEQ ID NO. 6) are evaluated.

5. A method for screening, predicting or monitoring benign prostate hyperplasia (BPH), comprising detecting the expression level of any combination of from two to ten genes, or the respective protein products thereof, selected from CXCL13 (SEQ ID NO. 1), NELL2 (SEQ ID NO. 2), SH3 (SEQ ID NO. 3), FBLN1 (SEQ ID NO. 4), BMP5 (SEQ ID NO. 5), WNT2 (SEQ ID NO. 6), X11L (SEQ. ID NO. 7), TIMP3 (SEQ ID NO. 8), JM27 (SEQ ID NO. 9) and COL4A2 (SEQ ID NO. 10).

6. The method of claim 5, wherein the combination comprises CXCL13 (SEQ. ID NO. 1) and BMP5 (SEQ. ID NO. 5), wherein overexpression of CXL13 (SEQ ID NO. 1) and BMP5 (SEQ ID NO. 5) indicates the presence of BPH.

7. The method of claim 6, further comprising evaluating the expression level of NELL2 (SEQ ID NO. 2), SH3 (SEQ ID NO. 3), FBLN1 (SEQ ID NO. 4), WNT2 (SEQ ID NO. 6), X11L (SEQ ID NO. 7), TIMP3 (SEQ ID NO. 8), JM27 (SEQ ID NO. 9) or COL4A2 (SEQ ID NO. 10).

8. A method for detecting the presence of benign prostate hyperplasia (BPH) and the absence of prostate cancer in a subject comprising detecting the expression level of CXL13 (SEQ ID NO. 1) and BMP5 (SEQ ID NO. 5), or the respective protein products thereof, wherein overexpression of CXL13 (SEQ ID NO. 1) and BMP5 (SEQ ID NO. 5) indicates the presence of BPH and the absence of prostate cancer.

9. The method of claim 8, further comprising evaluating the expression level of NELL2 (SEQ ID NO. 2), SH3 (SEQ ID NO. 3), FBLN1 (SEQ ID NO. 4), WNT2 (SEQ ID NO. 6), X11L (SEQ ID NO. 7), TIMP3 (SEQ ID NO. 8), JM27 (SEQ ID NO. 9) or COL4A2 (SEQ ID NO. 10), or the respective protein products thereof.
